(19) United States
(12) Patent Application Publication
(10) Pub. No.: US 2009/0105959 A1
(43) Pub. Date: Apr. 23, 2009

(54) System and method for identification of individual samples from a multiplex mixture

(76) Inventors: Michael S. Braverman, New Haven, CT (US); Jan Fredrik Simons, San Francisco, CA (US); Maithreyan Srinivasan, Mountain View, CA (US); Gregory S. Turenchalk, New Haven, CT (US)

Correspondence Address: Ivor R. Elrifi;Mintz, Levin, Cohn, Ferris, Glovsky and Popeo, P.C, 666 Third Avenue - 24th Floor, New York, NY, 10017, US

(21) Appl. No.: 12/156,242

(22) Filed: May 29, 2008

Related U.S. Application Data
(60) Provisional application No. 60/941,381, filed on Jun. 1, 2007.

(51) Int. Cl. G01N 33/48

(52) U.S. Cl. 702/19

(57) ABSTRACT

An embodiment of an identifier element for identifying an origin of a template nucleic acid molecule is described that comprises a nucleic acid element comprising a sequence composition that enables detection of an introduced error in sequence data generated from the nucleic acid element and correction of the introduced error, where the nucleic acid element is constructed to couple with the end of a template nucleic acid molecule and identifies an origin of the template nucleic acid molecule.

### CROSS-REFERENCE TO RELATED APPLICATIONS

The present application is related to and claims priority from U.S. Provisional Patent Application Ser. No. 60/941,381, titled “System and Method for Identification of Individual Samples from a Multiplex Mixture”, filed Jun. 1, 2007, which is hereby incorporated by reference herein in its entirety for all purposes.

Each of the applications and patents cited in this text, as well as each document or reference cited in each of the applications and patents (including during the prosecution of each issued patent; “application cited documents”), and each of the U.S. and foreign applications or patents corresponding to and/or claiming priority from any of these applications and patents, and each of the documents cited or referenced in each of the application cited documents, are hereby expressly incorporated herein by reference. More generally, documents or references are cited in this text, either in a Reference List before the claims, or in the text itself; and, each of these documents or references (“herein-cited references”), as well as each document or reference cited in each of the herein-cited references (including any manufacturer's specifications, instructions, etc.), is hereby expressly incorporated herein by reference. Documents incorporated by reference into this text may be employed in the practice of the invention.

### FIELD OF THE INVENTION

The present invention relates to the fields of molecular biology and bioinformatics. More specifically, the invention relates to associating a unique identifier (UID) element, which is sometimes also referred to as a multiplex identifier (MID), with one or more nucleic acid elements derived from a specific sample, combining the associated elements from the sample with associated elements from one or more other samples into a multiplex mixture of said samples, and identifying each identifier and its associated sample from data generated by what are generally referred to as “Sequencing” techniques.

### BACKGROUND OF THE INVENTION

There are a number of “sequencing” techniques known in the art amenable for use with the presently described invention such as, for instance, techniques based upon what are referred to as Sanger sequencing methods commonly known to those of ordinary skill in the art that employ termination and size separation techniques. Other classes of powerful high throughput sequencing techniques for determining the identity or sequence composition of one or more nucleotides in a nucleic acid sample include what are referred to as “Sequencing-by-synthesis” techniques (SBS), “Sequencing-by-Hybridization” (SBH), or “Sequencing-by-Ligation” (SBL) techniques. Of these, SBS methods provide many desirable advantages over previously employed sequencing methods that include, but are not limited to the massively parallel generation of a large volume of high quality sequence information at a low cost relative to previous techniques. The term “massively parallel” as used herein generally refers to the simultaneous generation of sequence information from many different template molecules in parallel where the individual template molecule or population of substantially identical template molecules are separated or compartmentalized and simultaneously exposed to sequencing processes which may include a iterative series of reactions thereby producing an independent sequence read representing the nucleic acid composition of each template molecule. In other words, the advantage includes the ability to simultaneously sequence multiple nucleic acid elements associated with many different samples or different nucleic acid elements existing within a sample.

Typical embodiments of SBS methods comprise the stepwise synthesis of a single strand of polynucleotide molecule complementary to a template nucleic acid molecule whose nucleotide sequence composition is to be determined. For example, SBS techniques typically operate by adding a single nucleic acid (also referred to as a nucleotide) species to a nascent polynucleotide molecule complementary to a nucleic acid species of a template molecule at a corresponding sequence position. The addition of the nucleic acid species to the nascent molecule is generally detected using a variety of methods known in the art that include, but are not limited to what are referred to as pyrosequencing or fluorescent detection methods such as those that employ reversible terminators or energy transfer labels including fluorescent resonant energy transfer dyes (FRET). Typically, the process is iterative until a complete (i.e. all sequence positions are represented) or desired sequence length complementary to the template is synthesized.

Further, as described above many embodiments of SBS are enabled to perform sequencing operations in a massively parallel manner. For example, some embodiments of SBS methods are performed using instrumentation that automates one or more steps or operation associated with the preparation and/or sequencing methods. Some instruments employ elements such as plates with wells or other type of microreactor configuration that provide the ability to perform reactions in each of the wells or microreactors simultaneously. Additional examples of SBS techniques as well as systems and methods for massively parallel sequencing are described in U.S. Pat. Nos. 6,274,320; 6,258,568; 6,210,891, 7,211,390; 7,244,559; 7,264,929; 7,335,762; and 7,323,305 each of which is hereby incorporated by reference herein in its entirety for all purposes; and U.S. patent application Ser. No. 11/195,254, which is hereby incorporated by reference herein in its entirety for all purposes.

It may also be desirable in some embodiments of SBS, to generate many substantially identical copies of each template nucleic acid element that for instance, provides a stronger signal when one or more nucleotide species is incorporated in each nascent molecule in a population comprising the copies of a template nucleic acid molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above), isothermal amplification techniques, and Polymerase Chain Reaction (PCR) methods, each of the techniques are applicable for use with the presently described invention. One PCR technique that is particularly amenable to high throughput applications include what are referred to as emulsion PCR methods.

Typical embodiments of emulsion PCR methods include creating stable emulsion of two immiscible substances and are resistant to blending together where one substance is dispersed within a second substance. The emulsions may include droplets suspended within another fluid and are sometimes also referred to as compartments, microcapsules, microreactors, microenvironments, or other name commonly used in the related art. The droplets may range in size depending on the composition of the emulsion components and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Thermo cycling operations typical of PCR methods may be executed using the droplets to amplify an encapsulated nucleic acid template resulting in the generation of a population comprising many substantially identical copies of the template nucleic acid. Also in the present example, some or all of the described droplets may further encapsulate a solid substrate such as a bead for attachment of nucleic acids, reagents, labels, or other molecules of interest.

Embodiments of an emulsion useful with the presently described invention may include a very high density of droplets or microcapsules enabling the described chemical reactions to be performed in a massively parallel way. Additional examples of emulsions and their uses for sequencing applications are described in U.S. patent application Ser. Nos. 10/861,930; 10/866,392; 10/767,899; 11/045,678 each of which are hereby incorporated by reference herein in its entirety for all purposes.

Those of ordinary skill in the related art will appreciate that advantages provided by the massively parallel nature of the amplification and sequencing methods described herein may be particularly to amenable for processing what may be referred to as a “Multiplex” sample. For example, a multiplex composition may include representatives from multiple samples such as samples from multiple individuals. It may be desirable in many applications to combine multiple samples into a single multiplexed sample that may be processed in one operation as opposed to processing each sample separately. Thus the result may typically include a substantial savings in reagent, labor, and instrument usage and cost as well as a significant savings in processing time invested. The described advantages of multiplex processing become more pronounced as the numbers of individual samples increase. Further, multiplex processing has application in research as well as diagnostic contexts. For example, it may be desirable in many applications to employ a single multiplexed sample in an amplification reaction and subsequently processing the amplified multiplex composition in a single sequencing run.

One problem associated with processing a multiplex composition then becomes identifying the association between each sample of origin and the sequence data generated from a template molecule derived from said sample. A solution to this problem includes associating an identifier such as a nucleic acid sequence that specifically identifies the association of each template molecule with its sample of origin. An advantage of this solution is that the sequence information of the associated nucleic acid sequence is embedded in the sequence data generated from the template molecule and may be bioinformatically analyzed to associate the sequence data with its sample of origin.

Previous studies have described associating nucleic acid sequence identifiers with 5′ primers coupled with target sequences for multiplex processing. One such study is that of Binladen et al. (Binladen J, Gilbert M T P, Bollback J P, Panitz F, Bendixen C (2007) The use of coded PCR Primers Enables High-Throughput Sequencing of Multiple Homolog Amplification Products by Parallel 454 Sequencing. PLoS ONE 2 (2): e197.doi:10.1371/journal.pone.0000197 (published online Feb. 14, 2007, which is hereby incorporated by reference herein in its entirety for all purposes). As mentioned above, Binladen et al. describe associating short sequence identifiers with target sequences to be processed in a multiplex sample producing sequence data that is subsequently bioinformatically analyzed to associate the short identifiers with their sample of origin. However, there are limitations to simply attaching a nucleic acid identifier of generic sequence composition to a template molecule and identifying the sequence of said identifier in the generated sequence data. Of primary concern is the introduction of error into the sequence data from various mechanisms. Such mechanisms typically work in combination with each other and are generally not individually identifiable from the sequence data. Thus because of introduced error, an end user may not be able to identify the association between the sequence data with its sample of origin, or possibly worse fail to identify that an error has occurred and mis-assign sequence data to a sample of origin that is incorrect.

There are two important sources of error introduction to consider, although other sources may also exist. First is error introduced by the sequencing operation that may in some cases be referred to a “flow error”. For example, flow error may include polymerase errors that include incorporation of an incorrect nucleotide species by a polymerase enzyme. A sequencing operation may also introduce what may be referred to as phasic synchrony error that include what are referred to as “carry forward” and “incomplete extension” (the combination of phasic synchrony error is sometimes referred to as CAFIE error). Phasic synchrony error and methods of correction are further described in PCT Application Serial No. US2007/004187, titled “System and Method for Correcting Primer Extension Errors in Nucleic Acid Sequence Data”, filed Feb. 15, 2007 which is hereby incorporated by reference herein in its entirety for all purposes.

Second is error introduced from processes that are independent of the sequencing operations such as primer synthesis or amplification error. For example, oligonucleotide primers synthesized for PCR may include one or more UID elements of the presently described invention, where error may be introduced in the synthesis of the primer/UID element that is then employed as a sequencing template. High fidelity sequencing of the UID element faithfully reproduces the synthesized error in sequence data. Also in the present example, polymerase enzymes commonly employed in PCR methods are known for having a measure of replication error, where for instance an error in replication may be introduced by the polymerase in 1 of every 10,000; 100,000; or 1,000,000 bases amplified.

Therefore, it is significantly advantageous to employ unique identifiers that are 1) resistant to error introduction; 2) enable detection of introduced error; and 3) enable correction of introduced error. The presently described invention addresses these problems and provides systems and methods for associating unique identifiers that provide better recognition and identification characteristics resulting in improved data quality and experimental efficiency.

### SUMMARY OF THE INVENTION

Embodiments of the invention relate to the determination of the sequence of nucleic acids. More particularly, embodiments of the invention relate to methods and systems for correcting errors in data obtained during the sequencing of nucleic acids and associating the nucleic acids with their origin.

An embodiment of an identifier element for identifying an origin of a template nucleic acid molecule is described that comprises a nucleic acid element comprising a sequence composition that enables detection of an introduced error in sequence data generated from the nucleic acid element and correction of the introduced error, where the nucleic acid element is constructed to couple with the end of a template nucleic acid molecule and identifies an origin of the template nucleic acid molecule.

Also, an embodiment of a method for identifying an origin of a template nucleic acid molecule is described that comprises the steps of identifying a first identifier sequence from sequence data generated from a template nucleic acid molecule; detecting an introduced error in the first identifier sequence; correcting the introduced error in the first identifier sequence; associating the corrected first identifier sequence with a first identifier element coupled to the template molecule; and identifying an origin of the template molecule using the association of the corrected first identifier sequence with the first identifier element.

In some implementations, the method further comprises the steps of identifying a second identifier sequence from the sequence data generated from the template nucleic acid molecule; detecting an introduced error in the second identifier sequence; correcting the introduced error in the second identifier sequence; associating the corrected second identifier sequence with a second identifier element coupled with the template nucleic acid molecule; and identifying an origin of the template nucleic acid molecule using the association of the corrected second identifier sequence with the second identifier element combinatorially with the association of the corrected first identifier sequence with the first identifier element.

Further, an embodiment of a kit for identifying an origin of a template nucleic acid molecule is described that comprises a set of nucleic acid elements each comprising a distinctive sequence composition that enables detection of an introduced error in sequence data generated from each nucleic acid element and correction of the introduced error, wherein each of the nucleic acid elements is constructed to couple with the end of a template nucleic acid molecule and identifies the origin of the template nucleic acid molecule.

In addition, an embodiment of a computer comprising executable code stored in system memory is described where the executable code performs a method for identifying an origin of a template nucleic acid molecule comprising the steps of identifying an identifier sequence from sequence data generated from a template nucleic acid molecule; detecting an introduced error in the identifier sequence; correcting the introduced error in the identifier sequence; associating the corrected identifier sequence with an identifier element coupled with the template molecule; and identifying an origin of the template molecule using the association of the corrected identifier sequence with the identifier element.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 160 appears first in FIG. 1). All of these conventions, however, are intended to be typical or illustrative, rather than limiting.

FIG. 1 is a functional block diagram of one embodiment of a sequencing instrument and computer system amenable for use with the presently described invention;

FIG. 2A is a simplified graphical representation of one embodiment of an adaptor element amenable for use with genomic libraries comprising a UID component;

FIG. 2B is a simplified graphical representation of one embodiment of an adaptor element amenable for use with amplicons comprising a UID component; and

FIG. 3 is a simplified graphical representation of one embodiment of computed error balls representing compatibility of UID elements of different sequence composition.

### DETAILED DESCRIPTION OF THE INVENTION

As will be described in greater detail below, embodiments of the presently described invention include systems and methods for associating a unique identifier hereafter referred to as a UID element with one or more nucleic acid molecules from a sample. The UID elements are resistant to introduced error in sequence data, and enable detection and correction of error. Further, the invention includes combining or pooling those UID associated nucleic acid molecules with similarly UID associated (sometimes also referred to as “labeled”) nucleic acid molecules from one or more other samples, and sequencing each nucleic acid molecule in the pooled sample to generate sequence data for each nucleic acid. The presently described invention further includes systems and methods for designing the sequence composition for each UID element and analyzing the sequence data of each nucleic acid to identify an embedded UID sequence code and associating said code with the sample identity.

### a. General

The terms “flowgram” and “pyrogram” may be used interchangeably herein and generally refer to a graphical representation of sequence data generated by SBS methods.

Further, the term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecule.

The term “flow” as used herein generally refers to a serial or iterative cycle of addition of solution to an environment comprising a template nucleic acid molecule, where the solution may include a nucleotide species for addition to a nascent molecule or other reagent such as buffers or enzymes that may be employed to reduce carryover or noise effects from previous flow cycles of nucleotide species.

The term “flow cycle” as used herein generally refers to a sequential series of flows where a nucleotide species is flowed once during the cycle (i.e. a flow cycle may include a sequential addition in the order of T, A, C, G nucleotide species, although other sequence combinations are also considered part of the definition). Typically the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule.

The term “monomer repeat” or “homopolymers” as used herein generally refers to two or more sequence positions comprising the same nucleotide species (i.e. a repeated nucleotide species).

The term “homogeneous extension”, as used herein, generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products such as those produced from Polymerase Chain Reaction or Ligase Chain Reaction techniques.

The term “keypass” or “keypass mapping” as used herein generally refers to a nucleic acid “key element” associated with a template nucleic acid molecule in a known location (i.e. typically included in a ligated adaptor element) comprising known sequence composition that is employed as a quality control reference for sequence data generated from template molecules. The sequence data passes the quality control if it includes the known sequence composition associated with a Key element in the correct location.

The term “blunt end” or “blunt ended” as used herein generally refers to a linear double stranded nucleic acid molecule having an end that terminates with a pair of complementary nucleotide base species, where a pair of blunt ends are always compatible for ligation to each other.

Some exemplary embodiments of systems and methods associated with sample preparation and processing, generation of sequence data, and analysis of sequence data are generally described below, some or all of which are amenable for use with embodiments of the presently described invention. In particular the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample must be prepared and processed from its raw form into template molecules amenable for high throughput sequencing. The processing methods may vary from application to application resulting in template molecules comprising various characteristics. For example, in some embodiments of high throughput sequencing it is preferable to generate template molecules with a sequence or read length that is at least the length a particular sequencing method can accurately produce sequence data for. In the present example, the length may include a range of about 25-30 base pairs, about 30-50 base pairs, about 50-100 base pairs, about 100-200 base pairs, about 200-300 base pairs, or about 350-500 base pairs, or other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids are employed that include what is referred to as nebulization or sonication. It will however, be appreciated that other methods of fragmentation such as digestion using restriction endonucleases may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements, unique identifiers that encode various associations such as with a sample of origin or patient, or other functional element. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In the present example, two sets of priming sequence regions (hereafter referred to as priming sequence A, and priming sequence B) may be employed for strand selection where only single strands having one copy of priming sequence A and one copy of priming sequence B is selected and included as the prepared sample. The same priming sequence regions may be employed in methods for amplification and immobilization where, for instance priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; and U.S. Provisional Application Ser. No. 60/941,381, titled “System and Method for Identification of Individual Samples from a Multiplex Mixture”, filed Jun. 1, 2007, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid such as a water based fluid suspended or dispersed in what may be referred to as a discontinuous phase within another fluid such as an oil based fluid. Further, some emulsion embodiments may employ surfactants that act to stabilize the emulsion that may be particularly useful for specific processing methods such as PCR. Some embodiments of surfactant may include non-ionic surfactants such as sorbitan monooleate (also referred to as Span™ 80), polyoxyethylenesorbitsan monooleate (also referred to as Tween™ 80), or in some preferred embodiments dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template or other type of nucleic acids, reagents, labels, or other molecules of interest.

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. patent application Ser. Nos. 10/861,930; 10/866,392; 10/767,899; 11/045,678 each of which are hereby incorporated by reference herein in its entirety for all purposes.

Also, an exemplary embodiment for generating target specific amplicons for sequencing is described that includes using sets of nucleic acid primers to amplify a selected target region or regions from a sample comprising the target nucleic acid. Further, the sample may include a population of nucleic acid molecules that are known or suspected to contain sequence variants and the primers may be employed to amplify and provide insight into the distribution of sequence variants in the sample.

For example a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (amplicons) is subsequently further amplified individually in separate reaction vessels such as an emulsion based vessel described above. The resulting amplicons (referred to herein as second amplicons), each derived from one member of the first population of amplicons, are sequenced and the collection of sequences, from different emulsion PCR amplicons, are used to determine an allelic frequency.

Some advantages of the described target specific amplification and sequencing methods include a higher level of sensitivity than previously achieved. Further, embodiments that employ high throughput sequencing instrumentation such as for instance embodiments that employ what is referred to as a PicoTiterPlate® array of wells provided by 454 Life Sciences Corporation, the described methods can be employed to sequence over 100,000 or over 300,000 different copies of an allele per run or experiment. Also, the described methods provide a sensitivity of detection of low abundance alleles which may represent 1% or less of the allelic variants. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005, which is hereby incorporated by reference herein in its entirety for all purposes.

Further, embodiments of sequencing may include Sanger type techniques, what is referred to as polony sequencing techniques, nanopore and other single molecule detection techniques, or reversible terminator techniques. As described above a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include but are not limited to mass tags and fluorescent or chemiluminescent labels. In typical embodiments, unincorporated nucleotides are removed, for example by washing. Further, in some embodiments the unincorporated nucleotides may be subjected to enzymatic degradation such as, for instance, degradation using the apyrase enzyme as described in U.S. Provisional Patent Application Ser. No. 60/946,743, titled System and Method For Adaptive Reagent Control in Nucleic Acid Sequencing, filed Jun. 28, 2007, which is hereby incorporated by reference herein in its entirety for all purposes. In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 150 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. patent application Ser. No. 11/448,462, titled “Paired end sequencing”, filed Jun. 6, 2006, and in U.S. Provisional Patent Application Ser. No. 60/026,319, titled “Paired end sequencing”, filed Feb. 5, 2008, each of which is hereby incorporated by reference herein in its entirety for all purposes.

Some examples of SBS apparatus may implement some or all of the methods described above may include one or more of a detection device such as a charge coupled device (i.e. CCD camera), a microfluidics chamber or flow cell, a reaction substrate, and/or a pump and flow valves. Taking the example of pyrophosphate based sequencing, embodiments of an apparatus may employ a chemiluminescent detection strategy that produces an inherently low level of background noise.

In some embodiments, the reaction substrate for sequencing may include what is referred to as a PicoTiterPlate® array (also referred to as a PTP® plate) formed from a fiber optics faceplate that is acid-etched to yield hundreds of thousands of very small wells each enabled to hold a population of substantially identical template molecules. In some embodiments, each population of substantially identical template molecule may be disposed upon a solid substrate such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. Further examples of apparatus and methods for performing SBS type sequencing and pyrophosphate sequencing are described in U.S. Pat. No. 7,323,305 and U.S. patent application Ser. No. 11/195,254 both of which are incorporated by reference above.

In addition, systems and methods may be employed that automate one or more sample preparation processes, such as the emPCR™ process described above. For example, microfluidic technologies may be employed to provide a low cost, disposable solution for generating an emulsion for emPCR processing, performing PCR Thermocycling operations, and enriching for successfully prepared populations of nucleic acid molecules for sequencing. Examples of microfluidic systems for sample preparation are described in U.S. Provisional Patent Application Ser. No. 60/915,968, titled “System and Method for Microfluidic Control of Nucleic Acid amplification and Segregation”, filed May 4, 2007, which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. Computers typically include known components such as a processor, an operating system, system memory, memory storage devices, input-output controllers, input-output devices, and display devices. It will be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Centrino®, Core™ 2, Itanium® or Pentium® processor made by Intel Corporation, a SPARC® processor made by Sun Microsystems, an Athalon™ or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP or Windows Vista®) from the Microsoft Corporation; the Mac OS X operating system from Apple Computer Corp. (such as 7.5 Mac OS X v10.4 “Tiger” or 7.6 Mac OS X v10.5 “Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example some commonly employed web browsers include Microsoft® Internet Explorer 7 available from Microsoft Corporation, Mozilla Firefox® 2 from the Mozilla Corporation, Safari 1.2 from Apple Computer Corp., or other type of web browser currently known in the art or to be developed in the future. Also, in the same or other embodiments an internet client may include, or could be an element of, specialized software applications enabled to access remote information via a network such as a data processing application for SBS applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

As described above, the presently described invention comprises associating one or more embodiments of a UID element having a known and identifiable sequence composition with a sample, and coupling the embodiments of UID element with template nucleic acid molecules from the associated samples. The UID coupled template nucleic acid molecules from a number of different samples are pooled into a single “Multiplexed” sample or composition that can then be efficiently processed to produce sequence data for each UID coupled template nucleic acid molecule. The sequence data for each template nucleic acid is de-convoluted to identify the sequence composition of coupled UID elements and association with sample of origin identified. For example, a multiplexed composition may include representatives from about 384 samples, about 96 samples, about 50 samples, about 20 samples, about 16 samples, about 10 samples, or other number of samples. Each sample may be associated with a different experimental condition, treatment, species, or individual in a research context. Similarly, each sample may be associated with a different tissue, cell, individual, condition, or treatment in a diagnostic context. Those of ordinary skill in the related art will appreciate that the numbers of samples listed above are for the purposes of example and thus should not be considered limiting.

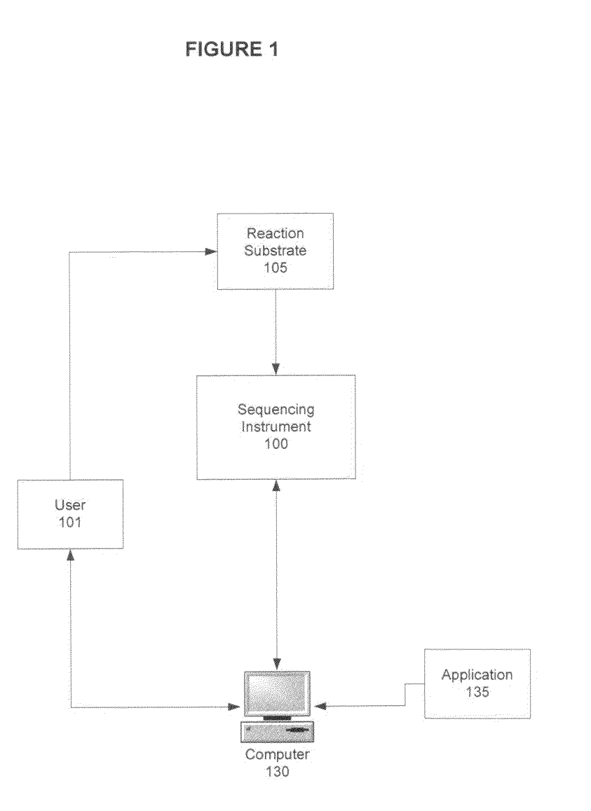

Typically, systems and methods are employed for processing samples to generate sequence data as well as for interpretation of the sequence data. FIG. 1 provides an illustrative example of sequencing instrument 100 employed to execute sequencing processes using reaction substrate 105 that for instance may include the PTP® plate substrate described above. Also illustrated in FIG. 1 is computer 130 that may for instance execute system software or firmware for processing as well as perform analysis functions. In the example of FIG. 1, computer 130 may also store application 135 in system memory for execution, where application 135 may perform some or all of the data processing functions described herein. It will also be understood that application 135 may be stored on other computer or server type structures for execution and perform some or all of its functions remotely communicating over networks or transferring information via standard media. For instance, processed target molecules in a multiplex sample may be loaded onto reaction substrate 105 by user 101 or some automated embodiment then sequenced in a massively parallel manner using sequencing instrument 100 to produce sequence data representing the sequence composition of each target molecule. Importantly, user 101 may include any user such as independent researcher, university, or corporate entity. In the present example, sequencing instrument 100, reaction substrate 105, and/or computer 130 may include some or all of the components and characteristics of the embodiments generally described above.

In preferred embodiments, the sequence composition of each UID element is easily identifiable and resistant to introduced error from sequencing processes. Some embodiments of UID element comprise a unique sequence composition of nucleic acid species that has minimal sequence similarity to a naturally occurring sequence. Alternatively, embodiments of a UID element may include some degree of sequence similarity to naturally occurring sequence.

Also, in preferred embodiments the position of each UID element is known relative to some feature of the template nucleic acid molecule and/or adaptor elements coupled to the template molecule. Having a known position of each UID is useful for finding the UID element in sequence data and interpretation of the UID sequence composition for possible errors and subsequent association with the sample of origin.

For example, some features useful as anchors for positional relationship to UID elements may include, but are not limited to the length of the template molecule (i.e. the UID element is known to be so many sequence positions from the 5′ or 3′ end), recognizable sequence markers such as a Key element (described in greater detail below) and/or one or more primer elements positioned adjacent to a UID element. In the present example, The Key and primer elements generally comprise a known sequence composition that typically does not vary from sample to sample in the multiplex composition and may be employed as positional references for searching for the UID element. An analysis algorithm implemented by application 135 may be executed on computer 130 to analyze generated sequence data for each UID coupled template to identify the more easily recognizable Key and/or primer elements, and extrapolate from those positions to identify a sequence region presumed to include the sequence of the UID element. Application 135 may then process the sequence composition of the presumed region and possibly some distance away in the flanking regions to positively identify the UID element and its sequence composition.

Also, as will be described in greater detail below in some embodiments the sequence data generated from each Key and/or one or more primer elements may be analyzed to determine a measure of the relative error rate for the sequencing run. The measure of error rate may then be employed in the analysis of the sequence data generated for the UID element. For example, if the error rate is excessive and is above a predetermined threshold it may also be assumed that a similar rate of error exists in the sequence data generated for the UID element, and thus the sequence data for the entire template may be filtered out as suspect. Further, in embodiments where a UID element is coupled to each end of a linear template molecule an error rate may be established for each end and asymmetrically analyzed. Importantly, it will be appreciated that in some embodiments, particularly sequencing technology capable of producing “long” read lengths (i.e. of about 100 base pairs or greater) the error rate in the sequence data may differ between the 5′ end and the 3′ end.

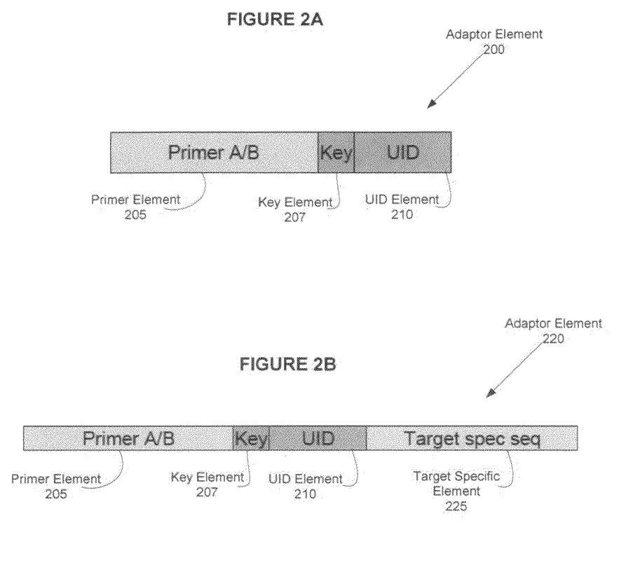

In preferred embodiments, a UID element is associated with an adaptor enabled to operatively couple with the end of a template nucleic acid molecule. In typical high throughput sequencing applications it is desirable that the template nucleic acid molecules are linear where an adaptor may be coupled to each end. FIGS. 2A and 2B provide illustrative examples of embodiments of adaptor composition for various applications comprising one or more UID elements. It will, however, be appreciated that various adaptor configurations may be employed for different amplification and sequencing strategies. FIG. 2A provides an illustrative example of adaptor element 200 that comprises an embodiment of an adaptor amenable for use with amplification and sequencing of Genomic Libraries. It will also be appreciated that adaptor element 200 may also be amenable for libraries of template molecules independently amplified with target specific sequences independently of the adaptor element described herein. Adaptor element 200 comprises several components that include primer 205, key 207, and UID 210. Also, FIG. 2B provides an illustrative example of one embodiment of adaptor 220 amenable for use with amplification and sequencing of Amplicons. Adaptor element 220 comprises several similar components to adaptor 200 that include primer 205, key 207, UID 210, with the addition of target specific element 225. It will be appreciated that the relative arrangement of components provided in FIGS. 2A and 2B are for illustrative purposes and should not be considered limiting.

In some alternative embodiments, the UID 210 elements are not associated with adaptor elements as described above. Rather, the UID 210 elements may be considered separate elements that may be independently coupled to an already adapted template molecule, or non-adapted template molecule. This strategy may be useful in some circumstances to avoid negative effects associated with a particular step or assay. For example, it may be advantageous in some embodiments to ligate the UID 210 elements to each population of substantially identical template molecules after copies have been produced from an amplification step. By coupling the UID elements to the adapted template molecules post-amplification, errors introduced by the amplification method are avoided. In the present example, PCR amplification methods that employ polymerases are known to have a certain rates of introduced error based, at least in part, upon the type of polymerase or polymerase blends (i.e. a blend may include a mixture of what may be referred to as a “high fidelity” polymerase and a polymerase with “proof reading” capability) employed and the number of cycles of amplification.

It will also be appreciated that multiple embodiments of adaptor 200 or 220 may be employed with each template molecule, such as one embodiment of adaptor 200 or 220 at each end of a linear template molecule prepared for sequencing. However, in some embodiments the positional arrangement of elements within adaptor 200 or 220 may be reversed (i.e. the elements of adaptor 200 or 220 are in a palindromic arrangement from the example illustrated in FIG. 2A or 2B) at the 3′ end relative the arrangement of elements in adaptor 200 or 220 at the 5′ end. For example, an embodiment of element 220 may be positioned on each end of substantially every template molecule from a library of amplicons in a multiplex composition, thus 2 embodiments of UID 210 may be employed in a combinatorial manner for identification which will be discussed in greater detail below.

Primer 205 may include a primer species (or a primer of a primer pair) such as is described above with respect to emulsion PCR embodiments (i.e. Primer A and Primer B). Also, primer 205 may include a primer species employed for an SBS sequencing reaction also as described above. Further, primer 205 may include what is referred to as a bipartite PCR/sequencing primer useable for both the emulsion PCR and SBS sequencing processes. Key 207 may include what may be referred to as a “discriminating key sequence” that refers to a short sequence of nucleotide species such as a combination of the four nucleotide species (i.e., A, C, G, T). Typically, key 207 may employed for quality control of sequence data, where for example key 207 may be located immediately adjacent primer 205 or within close proximity and include one of each of the four nucleotide species in a known sequence arrangement (i.e. TCAG). Therefore, the fidelity of the sequencing method should be represented in the sequence data for each of the 4 nucleotide species in key 207 and may pass quality control metrics if each of the 4 nucleotide species is faithfully represented. For example, an error for one of the nucleotide species represented in the sequence data generated from key 207 could indicate a problem in the sequencing process associated with that nucleotide species. Such error may be from mechanical failure of one or more components of sequencing instrument 100, low quality or supply of reagent, operating script error, or other source of systematic type error that may occur. Thus, if such systematic type error is detected in key 207 that sequence data generated for the run of that template molecule may not pass quality metrics and will typically be rejected.

The same discriminating sequence for key 207 can be used for an entire library of DNA fragments, or alternatively different sequence compositions may be associated with portions of the library for different purposes. Further examples of primer and key elements associated with primer 205 and key 207 are described in U.S. patent application Ser. No. 10/767,894, incorporated by reference above.

Target specific element 225 includes a sequence composition that specifically recognizes a region of a genome. For example, Target specific element 225 may be employed as a primer sequence to amplify and produce amplicon libraries of specific targeted regions for sequencing such as those found within genomes, tissue samples, heterogeneous cell populations or environmental samples. These can include, for example, PCR products, candidate genes, mutational hot spots, evolutionary or medically important variable regions. It could also be used for applications such as whole genome amplification with subsequent whole genome sequencing by using variable or degenerate amplification primers. Further examples describing the use of target specific sequences with bipartite primers are described in U.S. patent application Ser. No. 11/104,781, titled “Methods for determining sequence variants using ultra-deep sequencing”, filed Apr. 12, 2005, which is hereby incorporated by reference herein in its entirety for all purposes.

Some embodiments of UID 210 may be particularly amenable for use with relatively small numbers of sample associations in a multiplex sample. In particular, when there are only a small number of associations to identify in a multiplex sample, each sample is associated with a distinct implementation of UID 210 comprising a sequence composition that is sufficiently unique from each other as to enable easy detection and correction of introduced error. In some embodiments, groups of compatible UID 210 sequence elements are clustered into “sets” as will be described in greater detail below. For example, a set of UID 210 elements may include 14 members that may be employed to uniquely identify up to 14 associations with samples, where each member is associated with a single sample.

It will be appreciated that as the number of associations to identify grows, it becomes increasingly difficult to design distinct embodiments of UID 210 for each association that meet the design criteria and desired characteristics. In such cases, it may be advantageous to employ multiple UID 210 elements combinatorially to uniquely associate the template molecules with their sample of origin, where one embodiment of UID 210 may be positioned at each end of a linear template molecule. For example, the number of associations to identify between the sequence data generated from template molecules and the sample of origin may become too large to accommodate given the necessary design parameters and characteristics of UID 210. In particular, it is undesirable in many embodiments to employ a distinct UID element for each association when the number a samples would require a sequence length for UID 210 that is undesirably long for the design criteria that includes a specific number of flow cycle iterations and number of sequence positions taken up by the UID element. In the present example, in embodiments of sequencing technology that generate “long” read lengths UID 210 may comprise up to 10 sequence positions. Alternatively, other embodiments of sequencing technology may generate relatively short read lengths of about 25-50 sequence positions, and thus it is desirable that UID 210 is short in order to optimize the read length for the template molecule. In the present example, UID 210 may be designed for short read lengths comprising up to 4 sequence positions, up to 6 sequence positions, or up to 8 sequence positions, depending, at least in part, upon the application.

As described above, embodiments for design and implementation of UID 210 amenable for both small and large numbers of associations is to employ a “set” of UID 210 elements each meeting the preferred design criteria and characteristics. In some applications, such as the design of UID 210 elements with sequence composition that enable accurate error detection and correction features it is desirable to use the “set” strategy presently described. For example, as will be described in greater detail below the sequence composition for the UID elements in a set must be sufficiently distinct from each other in order to enable error detection and correction thereby limiting the compatible members available for a particular set. However, UID 210 members from multiple sets may be combinatorially employed with a template molecule where the members of each set are located at different relative positions and are thus easily interpretable.

In order to overcome the problems of a large number of associations to identify described above, two or more members from a set of UID 210 elements may be employed in a combinatorial manner. For example, a set of UID 210 elements may include 10, 12, 14, or other number of members comprising a 10-mer sequence length. In some embodiments, two UID 210 elements may be associated with each template molecule and used combinatorially to identify up to 144 different associations (i.e. 12 UID members for use with element 1 multiplied by 12 UID members for use with element 2 results in 144 possible combinations of UID elements 1 and 2 that may be employed to uniquely identify an association).

Those of ordinary skill in the related art will appreciate that alternative embodiments may be employed where each UID 210 element associated with a template molecule may include a subset of the total number of UID members from the set (i.e. use a portion of the members of the set). In other words, of the 12 members of a complete set, only 8 may be employed at one element position. There are a number of reasons why it may be desirable to use a subset of UID members that includes having a need for a smaller number of associations to identify (i.e. smaller number of combinations), physical or practical experimental conditions such as equipment or software limitations, or preferred combinations of UID members of a set in element positions. For instance, a first element may employ all 12 UID members from a set and a second element may employ a subset of 8 UID members from the same or different set yielding 96 possible combinations.

UID 210 elements used in combinatorial strategies may be configured in a variety of positional arrangements relative to the position of the template molecule. For example, a strategy that utilizes 2 UID 210 elements combinatorially to identify the association of each template molecule with its sample of origin may include a UID element positioned at each end of a linear template molecule (i.e. one UID 210 element at the 5′ end and another at the 3′ end). In the present example, each UID 210 element may be associated with an adaptor element, such as adaptor 200 or 220, employed in a target specific amplicon or genomic library sequencing strategy as discussed above. Thus, the sequence data associated with a template molecule would include the sequence composition of a UID element at each end of the amplicon. The combination of the UID elements may then be used to associate the sequence data with the sample of origin of the template molecule.

In some alternative embodiments, a UID 210 element may be incorporated in an adaptor element at each end of a linear template molecule as described above. However, the read length of the template molecule may be greater than the ability of the sequencing technology to handle. In such a case, the template molecule may be sequenced from each end independently (i.e. a separate sequencing run for each end), where the UID 210 element associated with the end may be employed as a single UID 210 identifier.

In addition it may be desirable in some embodiments to assign more that one UID 210 element per sample, or more than one combinations of UID 210 elements. Such a strategy may provide redundancy to protect against possible unintended biases introduced by various source, which could include the UID 210 element itself. For example, a sample with a population of template molecules may be sub-divided in sub-samples each using a distinctive UID 210 element for the association. In such a case, the redundancy of the different UID 210 elements for the same population of template molecules from a sample provides for greater confidence that the correct associations will be identified or if the error is too great to make a correct identification of the association with confidence.

As generally described above, embodiments of the presently described invention include one or more UID 210 elements operatively coupled to each template molecule for the purpose of identifying the association between the template molecule and the sequence data generated therefrom with a sample of origin. One or more embodiments of a UID element may be operatively coupled to one or more components of an adaptor and a template molecule using a variety of methods known in the art that include but are not limited to ligation techniques. Methods for ligating nucleic acid molecules to one another are generally known in the art and include employing a ligase enzyme for what is referred to as sticky end or blunt end ligation. Further examples of coupling adaptor elements to template molecules using ligation as described in U.S. patent application Ser. No. 10/767,894, titled “Method for preparing single-stranded DNA libraries”, filed Jan. 28, 2004; and U.S. Provisional Patent Application Ser. No. 60/031,779, titled “System and Method for Improved Processing of Nucleic Acids for Production of Sequencable Libraries” filed Feb. 27, 2008, each of which is hereby incorporated by reference herein in its entirety for all purposes). For example, a large template nucleic acid or whole genomic DNA sample may be fragmented by mechanical (i.e. nebulization, sonication) or enzymatic means (i.e. DNase I), the resulting ends of each fragment may be polished for compatibility with adaptor elements (i.e. polishing using what is referred to as an exonuclease, such as BAL32 nuclease or Mung Bean nuclease), and each fragment may be ligated to one or more adaptor elements (i.e. using T4 DNA ligase). In the present example, each adaptor element is directionally ligated to the fragment such as for instance by selective binding between the 3′ end of the adaptor and the 5′ end of the fragment.

In some embodiments, UID 210 elements may be provided to user 101 in the form of a kit, where the kit could include adaptors comprising incorporated UID 210 elements as illustrated in FIGS. 2A and 2B. Or, the kit could include UID 210 as independent elements that enable user 101 to incorporate as they desire.

As described above, embodiments of UID 210 should comprise a number of preferred characteristics or design criteria that include but are not limited to a) each UID element comprises a minimal sequence length requiring a minimal number of synthesis or flow cycles, b) each UID element comprises sequence distinctiveness, c) each UID element comprises resistance to introduced error, and d) each UID element does not interfere with amplification methods (such as PCR, or cloning into vectors).

Also, some embodiments of UID element design may also consider physical characteristics or design criteria of nucleic acids that include some or all of i) UID sequence composition selected to resist formation of what are referred to as “hairpins” (also referred to as a “hairpin loop” or “stem loop”) and “primer dimers”; ii) UID elements comprise preferred melting temperature (i.e. 40° C.) and/or Gibbs free energy (i.e. ΔG cutoff of −1.5) characteristics. Aspects of some of the desirable characteristics and their impact on UID design are described in greater detail below.

One important characteristic of a UID element is that it should include a minimal number of bases or sequence positions required to satisfy the needs of other characteristic requirements. For example, each UID element should comprise the minimum sequence length required to uniquely identify a desired number of associations between the template molecule/sequence data and their samples of origin. A desired number of associations may include identification of template molecules/sequence data associated with at least 12 different samples, at least 96 different samples, at least 384 different samples, or a greater number of samples that may be contemplated in the future. In other words the sequence length of the UID should be no longer than necessary in order to conserve the number of positions (i.e. what may be referred to as “sequence real estate”) of the read length for the template molecule. Further, the minimum sequence length should consume or require a minimum number of flow cycles of the set of nucleotide species to generate the sequence data for each UID element. Minimizing the number of nucleotide species flow cycles required to generate sequence data for the UID elements provides advantages in reagent cost, instrument usage (i.e. processing time), data quality, and read length. For instance, each additional flow cycle increases the probability of introducing CAFIE error, and reagent usage. In the present example, it is preferable that each 10-mer UID element require only 5 nucleotide species flow cycles to generate sequence data for each UID element.

Another important characteristic includes sequence distinctiveness of each UID element. The term “sequence distinctiveness” as used herein generally refers to a distinguishable difference between a plurality of UID sequences such that each sequence is easily recognizable from every other UID sequence that is the subject of comparison. In particular each UID element needs to comprise a measure of sequence distinctiveness that enables easy detection of introduced error and correction of some or all of the error. Further, it is generally preferable that each UID element be free of repetitive sequence composition and should not include a sequence composition recognized by restriction enzymes. In other words it is undesirable for UID elements to include consecutive monomers having the same composition of nucleotide species. For example, preferred embodiments of the sequence distinctiveness of each UID element enable detection of up to 3 sequence positions with introduced errors and correction of up to 2 sequence positions with introduced errors in a 10-mer element (i.e. 10 total sequence positions). Those of ordinary skill will appreciate that the introduced error may include what are referred to as “insertions”, “deletions”, “substitutions”, or some combination thereof (i.e. a combination of an insertion and deletion at the same sequence position will appear to be a substitution and would be counted as a single error event). Also, the level of error detection and correction may depend, at least in part, upon the sequence length of the UID element. Further, introduced errors outside (i.e. upstream or downstream) of UID 210 may have effects on the interpretation of sequence composition for UID 210. This will be discussed further below in the context of decoding or analysis of sequence data for UID identification.

A further characteristic that is also desirable comprises resistance to introduced error. For example, monomer repeats in nucleic acid sequence such as that of the template molecule or other sequence elements may cause errors in a sequence read. The error may include an over or under representation or call of the number of repeated monomers. It is therefore desirable that the UID elements do not begin or end with the same nucleotide species as the adjacent monomer of a neighboring sequence element (i.e. creating monomer repeats between sequence elements or components). In the present example, a neighboring sequence element, such as key 207 illustrated in FIGS. 2A and 2B, may end with a “G” nucleotide species. Therefore, a UID element such as UID 210, should not begin with the same “G” nucleotide species to avoid the increased possibility introduced error from the repeated “G” species.

Another source of error that is particularly relevant in SBS contexts, include what are referred to as “carry forward” or “incomplete extension” effects (sometimes referred to as CAFIE effects). For example, a small fraction of template nucleic acid molecules in each amplified population of a nucleic acid molecule from a sample (i.e. a population of substantially identical copies amplified from a nucleic acid molecule template) loses or falls out of phasic synchronism with the rest of the template nucleic acid molecules in the population (that is, the reactions associated with the fraction of template molecules either get ahead of, or fall behind, the other template molecules in the sequencing reaction run on the population). Additional description of CAFIE mechanisms and methods of correcting CAFIE error are further described in PCT Application Serial No US2007/004187, titled “System and Method For Correcting Primer Extension Errors in Nucleic Acid Sequence Data”, filed Feb. 15, 2007, which is hereby incorporated by reference herein in its entirety for all purposes.

Also, it will be appreciated that some types of error may occur at higher frequency than other types and/or have greater consequences than other types of error. For example, deletion error may have more significant impact than substitution error. It is therefore advantageous to design each UID element so that it is weighted more heavily to deal with the more frequent or more deleterious types of error.

As stated previously, it is not typically desirable to randomly or non-selectively design the sequence composition of UID elements. An illustrative example of two improperly designed UID elements and the potential for problems with error detection/correction using such UID elements is presented in Table 1.

TABLE 1

| | | |
|---|---|---|
| UID Element 1 | Generated UID Sequence | UID Element 2 |

| | | |
|---|---|---|
| A [symbol] TGA | A [symbol] TGA | AGCGA |
| (SEQ ID NO: 1) | (SEQ ID NO: 2) | (SEQ ID NO: 3) |

In the example of table 1, it is apparent that the UID sequence represented as generated UID sequence contains an error (i.e. the presence of at least one error is detected) if either UID element 1 or 2 is the original sequence element. However, it is not clear from the sequence composition of the Generated UID sequence whether UID element 1 or UID element 2 was the actual UID element because a single error in either could result in the generated sequence. In other words, it is possible that one error was introduced in UID element 1 transforming the “C” nucleotide species at the second position to a “G” species. It is also possible that one error was introduced in UID element 2 transforming the “C” nucleotide species at the third position to a “T” species. Given the sequence information, the error is detected but it is not possible to infer which UID element was the original element and thus cannot be corrected. Therefore, the association of the generated UID sequence with either UID element 1 or 2 cannot be positively made, and thus the sample of origin for the template molecule coupled to one of the UID elements cannot be identified and the generated sequence information may need to be thrown out. In other words, the design of UID elements 1 and 2 are not sufficiently distinct from each other to recover from the described type of introduced error.

The potential result of poor UID design is further exemplified in Table 2.

TABLE 2

| UID Element 1 | UID Element 2 |
|---|---|
| CTACC (SEQ ID NO: 4) | CTGCC (SEQ ID NO: 5) |

The example of Table 2 provides an even clearer picture of the potential consequences where a substitution event in UID element 1 of an A nucleotide species at the third position to a G nucleotide species, which is one of the most common types of error introduced by PCR processes, results in an exact match with the sequence composition of UID 210 element. Thus the poor UID 210 design results in an undetectable error that would likely result in the mis-assignment of the sequence data to a sample of origin.

Various methods may be employed to design UID elements comprising sequence composition that meets the necessary design criteria. Also, application 135 illustrated in FIG. 1 may be employed for designing UID 210 using some or all of the methods described herein. For example, “Brute Force” methods may be employed that compute every possible sequence composition for a given length and the possible conflicts with other sequence composition given a set of parameters associated with the design criteria. In the present example, the sequence composition of 10 mer UID elements may be computed for detection of up to 3 sequence positions with introduced errors and correction of up to 2 sequence positions with introduced errors.

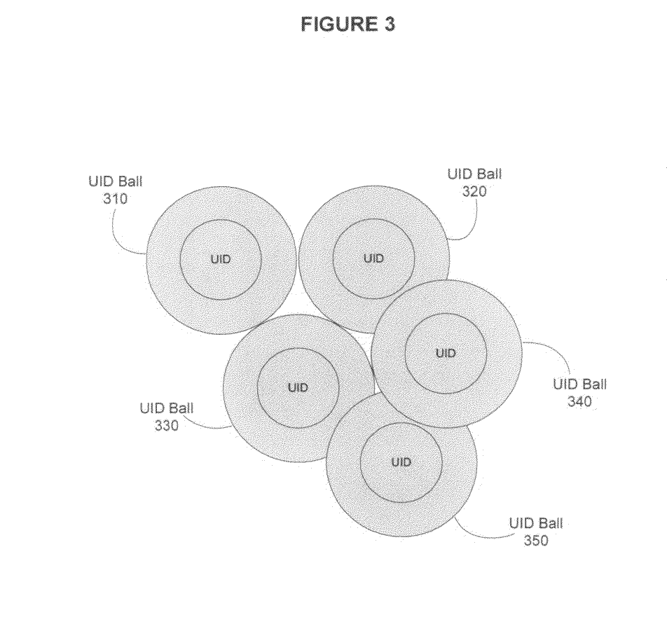

Design of a preferred sequence composition for members of a set of UID 210 elements meeting the most stringent design criteria given the characteristics described above presents a computational challenge. Mathematical methods known to those of skill in the art may be applied to compute the possible sequence composition for members of a set given the design constraints. For example, mathematical transformations of all possible combinations of sequence composition may be computed given the design constraints to generate what may be referred to as “Error Balls” or “Error Clouds” to determine the potential compatibility of each UID element with the other members in a set. Compatibility of sequence composition for potential UID elements may be visually illustrated as non-overlapping error balls. For example, FIG. 3 provides an illustrative representation of what may be referred to as “space potential” for computed error balls for UID 310, UID 320, UID 330, UID 340, and UID 350 comprising some or all of the design criteria described above such as number of flow cycles, and sequence length requirements. As illustrated in FIG. 3 the error balls for UID 310, UID 320, and UID 330 do not overlap and thus represent sequence composition of compatible UID 210 elements. Further, UID 340 overlaps with UID 320 and UID 350 representing a sequence composition for a UID element that is not compatible. However UID 340 does not overlap with UID 310 and UID 330 and thus represents compatible sequence composition for each non-overlapping UID element.

Alternatively, a more computationally efficient approach may be employed that uses what is referred to in the art as “Dynamic Programming” techniques. The term “Dynamic Programming” as used herein generally refers to methods for solving problems that comprise overlapping sub-problems and optimal structure. Dynamic programming techniques are typically substantially more computationally efficient than methods with no a priori knowledge.

Some embodiments of dynamic programming technique include computing what may be referred to as the “minimum edit distance” for strings of characters such as strings of nucleic acid species. In other words, each UID member element in a set may be considered a string of characters representing the nucleic acid species composition. The term “minimum edit distance” as used herein generally refers to the minimum number of point mutations required to change a first string into a second string. Further, the term “point mutation” as used herein generally refers to and includes a change of character composition at a location in a string referred to as a substitution of a character for another in a string; an insertion of a character into a string; or a deletion of a character from a string. For example, the minimum edit distance may be computed for each potential member of a set of UID 210 elements against all other members of the set. Subsequently the minimum edit distances may be compared and members of the set of UID 210 elements selected based, at least in part, upon each member of the set having a sufficiently high minimum edit distance from all other members to meet the specified criteria. Systems and methods for computing minimum edit distance are well known to those of ordinary skill in the related art and may be implemented in a number of ways.

Another important aspect of the presently described invention is directed to the analysis of sequence data to “decode” or identify the UID 210 sequence elements within the data. In some embodiments an algorithm may be implemented in computer code as application 135 that processes the sequence data from each run and identify UID 210 as well as perform any error detection or corrections functions. It is important to recognize that methods of error detection and correction in strings of information have been employed in the computer arts particularly in the area of electronically stored and transmitted data. For example, the problem of “inversion” of bits of data from one form into another occurs when data is transmitted over networks or stored in electronic media. The inversion of bits presents a problem with respect to the integrity of stored or transmitted data and is analogous to the presently described substitution type of error. Methods of detection and correction of inversion error is described in J. F. Wakerly, “Detection of unidirectional multiple errors using low cost arithmetic codes,” IEEE Trans. Comput., vol. C-24, pp. 210-212, February 1975; and J. F. Wakerly, Error Detecting Codes, Self-Checking Circuits and Applications. Amsterdam, The Netherlands: North-Holland, 1978, both of which are hereby incorporated by reference herein in their entireties for all purposes.

However, the methods of detecting and correcting inversion error described above are not applicable to the problem of error detection and correction in sequence data and more specifically errors in UID elements. Importantly, the problem in sequence data is substantially more complex because it deals with the problems of substitutions and deletions as well as substitutions that create phasing problems and complicate the interpretation of information at each sequence position.

As described above, UID 210 may be located at a known position relative to other easily identifiable elements such as primer 205, key 207, the 5′ or 3′ end of the sequence, etc. However, just as introduced error within UID 210 has deleterious effects, error outside of the region of the UID 210 element may also affect the efficiency of identifying each UID 210 element. Further, some types of error outside of the region defined by UID 210 may contribute to and count as errors within UID 210 sequence. For example, insertion events may occur and be represented in the sequence data preceding (i.e. upstream of) UID 210 element that may be difficult to interpret. In the present example, an insertion event could include the insertion of one or more G nucleotide species bases at the end of key 207 comprising a TCAG sequence composition as may occur when a nucleotide species at a sequence position is “overcalled”. However, an application that interprets the data will not know that it is an insertion event and cannot rule out the possibility of a substitution event that provided a G nucleotide in place of a different nucleotide species at the first sequence position of UID 210. In other words, the error outside of UID 210 will force the algorithm to decide if the error is an insertion that shifts where it should look for the first sequence position of UID 210 or whether it is a substitution event.

Continuing the example from above, an algorithm or user may look for the UID 210 element immediately adjacent to another known element such as key 207 as illustrated in FIGS. 2A and 2B, but the insertion of one base between key 207 and UID 210 may typically be assigned as belonging to UID 210 (counts as a first insertion error). Additionally, the algorithm or user expects UID 210 to be a certain length (i.e. 10 sequence positions) and thus truncates the last sequence position of the actual UID element because of the first insertion (counts as a second deletion error). Thus, it is clear that errors outside of the UID region can have substantial effect on finding and interpreting the sequence composition of UID 210.

In some embodiments, errors outside of the region defined by UID 210 may be particularly troublesome at the 3′ end of a nascent molecule. For example, some embodiments of SBS sequence from 5′ to 3′ ends (i.e. adding nucleotide species to 3′ end of nascent molecule) where cumulative errors (such as CAFIE type error described above) and the rate of introduced error may be increasingly higher as the sequence run gets longer at the 3′ end. Thus, it may be more practical and effective to use certain assumptions rather than stringent criteria to identify UID 210. Also as described above, assumptions used for the 5′ may be different than assumptions employed for the 3′ end and may be referred to as “Asymmetric”. For example, it may be assumed that there will never be more than 3 sequence position errors present at the 5′ end which would be consistent with empirical evidence. However, in the present example at the 3′ end it may be assumed that there will never be more than 4 sequence position errors due to the increased possibility of error at the 3′ end. Because of the asymmetric difference in detectable error at each end, it may also be inferred that the amount of that error that is correctable may also be different. In the present example, the correctable error at the 5′ end may be 2 sequence positions as described above, however the correctable error at the 3′ end may only be 1 sequence position. Also, further assumptions may be employed at the 3′ end that may not be employed for the 5′ end. Such an assumption could include the existence of one or more “no called” positions in close proximity to UID 210.

In the present example, an embodiment of adaptor element 200 or 220 is present at the 3′ end of a template nucleic acid in a palindromic arrangement to that illustrated in FIG. 2A or 2B (as described above). It will be appreciated however, that the present example refers to a difference in the arrangement of elements and that the elements associated with each adaptor do not need to have the same composition (i.e. the 3′ end may include the sequence composition of a first UID element and the 5′ end may include a UID elements with different sequence composition). It will further be appreciated that some embodiments will not necessarily include the same composition of elements in each adaptor (i.e. an adaptor at the 5′ end may include a UID 210 element and the adaptor on the 3′ may not, or vice versa). Also, there may be inherent internal controls of the sequence quality of primer element 205 with respect to resistance to introduced error. For instance, error introduced into the sequence composition of primer 205 would negatively affect its hybridization qualities to its respective target and thus not be amplified in a PCR process and therefore not represented in populations of template molecule for sequencing. This inherent quality control of primer 205 is useful for finding UID 210, because the sequence composition of primer 205 is known and can be assumed to be substantially free of error with the exception of some sequencing related error. Also as described above, key element 207 is employed for quality control purposes and it also useful as a positional reference in the same context. Thus, in the present example primer 205 and/or key 207 may serve as easily identifiable anchor points of reference for identifying UID 210 using the known positional relationships between elements. For instance, a user or algorithm, such as an algorithm implemented by application 135, may look for UID 210 located immediately adjacent to key 207, or some known distance away, based, at least in part, upon the assumptions.

Furthermore, once a user or algorithm has identified the sequence composition of a putative UID 210 element, the step of error identification and correction occurs. Embodiments of the presently described invention compare the sequence composition of the putative UID 210 element against the sequence compositions of the UID 210 members in the set. A perfect match is associated with its sample of origin. If no perfect match is found, then the closest UID 210 elements having a sequence composition to the putative sequence are analyzed to determine possible insertion, deletion, or substitution errors that could have occurred. For example, the closest UID 210 element to the putative UID 210 element is identified or the putative UID 210 element is deemed to have too many errors. In the present example, the minimum edit distance may be computed between sequence composition of the putative UID 210 element against the sequence composition of all members of the UID 210 set or select members. The minimum edit distance may be computed using the parameters of detecting up to 3 sequence position errors with the possibility of correcting up to 2 sequence position errors. In the present example, the UID 210 member with the closest or shortest minimum edit distance to the putative UID 210 element given the parameter constraints (i.e. detection/correction) may be assigned as the sequence composition of the putative UID 210 element. Also, if the minimum edit distance calculation determines that 3 sequence position errors have occurred then, the putative UID 210 element may be assigned as unusable and not associated with a sample of origin.

Those of ordinary skill in the art will appreciate that when the UID 210 elements are employed in a combinatorial manner, each UID 210 element is typically independently analyzed. Then the combination of identified UID 210 elements may be compared against the known combinations assigned to samples of origin to identify the association of the sequence data and its specific sample of origin.

In preferred embodiments, a UID 210 finding algorithm is implemented using application 135 stored for execution on computer 130 as described above. Further, the same or other application may perform the step of associating the identified UID 210 from sequence data with the sample of origin and providing the results to a user via an interface and/or storing the results in electronic media for subsequent analysis or use.

### Example 1

### Design of UID Elements Considering a Limited Number of Design Constraints

The design of sequence composition for potential UID elements were computed considering detection, correction, and hairpin design constraints.

First a sequence length of 10 base pairs for each UID element were computed yielding 1,048,576 possible elements.

Next, of those possible elements UID elements were selected that have no monomer repeats, require only 5 flow cycles (20 flows) or less, do not begin with the “G” nucleotide species were computed yielding 34,001 possible elements.

A further step of filtering to exclude hairpins at a temperature of 40° C. with a ΔG=−1.5 yielded 26,278 possible elements.

Finally, 5,000 of those possible elements were selected randomly to search for compatible sets or clusters that could correct 2 sequence position errors and detect 3 sequence position errors, yielding:

32,999 sets of 12 members

3,625 sets of 13 members

24 sets of 14 members

### Example 2

### Exemplary Computer Code for Creating UID Sequence Elements

UIDCreate.java class file that runs a search using 1 of 3 techniques, comprising (1) based on error clouds, (2) based on edit distance, and (3) based on edit distance, with an additional efficiency strategy of using a “safety map” to precompute the edit distance which gives the software the ability to effectively look ahead in the search in advance of trying candidate selections.

It will be appreciated that the foregoing computer code is provided for the purposes of example, and that numerous alternative methods and code structures may be employed. It will also be appreciated that the exemplary code provided herein is not intended to execute as a stand alone application or to run perfectly without additional computer code or modification.

### Example 3

### Table of Computed UID Sequences, Cluster ID, and Flowgram Script

| | | | | | |
|---|---|---|---|---|---|
| | | Flowgram | | | |
| | Member | TACGTACGTACGTACGTACG | | UID | SEQ |
| Cluster Id | Count | (SEQ ID NO: 6) | UID | Length | ID NO |

| | | | | | |
|---|---|---|---|---|---|
| C1127176 | 14 | 01100101010110011010 | ACAGAGTGTC | 10 | 7 |
| C1127176 | 14 | 01111010100101010100 | ACGTCTGAGA | 10 | 8 |
| C1127176 | 14 | 01010111001001101010 | AGACGCACTC | 10 | 9 |
| C1127176 | 14 | 01001010110010101011 | ATCTATCTCG | 10 | 10 |
| C1127176 | 14 | 00110100111100111000 | CGATACGCGT | 10 | 11 |
| C1127176 | 14 | 00110011001110010011 | CGCGCGTGCG | 10 | 12 |
| C1127176 | 14 | 00111101010011010010 | CGTAGATAGC | 10 | 13 |
| C1127176 | 14 | 00111001101010101100 | CGTGTCTCTA | 10 | 14 |
| C1127176 | 14 | 00101010011001110110 | CTCACACGAC | 10 | 15 |
| C1127176 | 14 | 11101010010010111000 | TACTCATCGT | 10 | 16 |
| C1127176 | 14 | 11010011010011100100 | TAGCGATACA | 10 | 17 |
| C1127176 | 14 | 11001001110111001000 | TATGTAGTAT | 10 | 18 |
| C1127176 | 14 | 10101001001101101001 | TCTGCGACTG | 10 | 19 |
| C1127176 | 14 | 10010110010110100101 | TGACAGTCAG | 10 | 20 |
| C1127177 | 14 | 01101101001101010100 | ACTAGCGAGA | 10 | 21 |
| C1127177 | 14 | 01010111010011001100 | AGACGATATA | 10 | 22 |
| C1127177 | 14 | 01001010100101111010 | ATCTGACGTC | 10 | 23 |
| C1127177 | 14 | 01001001101011010011 | ATGTCTAGCG | 10 | 24 |
| C1127177 | 14 | 00110100111100111000 | CGATACGCGT | 10 | 25 |
| C1127177 | 14 | 00110011001110010011 | CGCGCGTGCG | 10 | 26 |
| C1127177 | 14 | 00111010011001010110 | CGTCACAGAC | 10 | 27 |
| C1127177 | 14 | 00111001101010101100 | CGTGTCTCTA | 10 | 28 |
| C1127177 | 14 | 11101010010101001001 | TACTCAGATG | 10 | 29 |
| C1127177 | 14 | 11010010011010101010 | TAGCACTCTC | 10 | 30 |
| C1127177 | 14 | 11001100111001100100 | TATATACACA | 10 | 31 |
| C1127177 | 14 | 10100100101110100101 | TCATCGTCAG | 10 | 32 |
| C1127177 | 14 | 10010101100100110110 | TGAGTGCGAC | 10 | 33 |
| C1127177 | 14 | 10011001010111011000 | TGTGAGTAGT | 10 | 34 |
| C1127178 | 14 | 01100110101010010110 | ACACTCTGAC | 10 | 35 |
| C1127178 | 14 | 01010101010101101001 | AGAGAGACTG | 10 | 36 |
| C1127178 | 14 | 01001111110010101000 | ATACGTATCT | 10 | 37 |
| C1127178 | 14 | 01001011101101010100 | ATCGTCGAGA | 10 | 38 |
| C1127178 | 14 | 00100110010111011100 | CACAGTAGTA | 10 | 39 |
| C1127178 | 14 | 00110100111100111000 | CGATACGCGT | 10 | 40 |
| C1127178 | 14 | 00110011001110010011 | CGCGCGTGCG | 10 | 41 |
| C1127178 | 14 | 00111001101010101100 | CGTGTCTCTA | 10 | 42 |
| C1127178 | 14 | 00101001110101001011 | CTGTAGATCG | 10 | 43 |
| C1127178 | 14 | 11101001010100110010 | TACTGAGCGC | 10 | 44 |
| C1127178 | 14 | 11010010101111001000 | TAGCTCGTAT | 10 | 45 |
| C1127178 | 14 | 11001100111001100100 | TATATACACA | 10 | 46 |
| C1127178 | 14 | 10110010011001101010 | TCGCACACTC | 10 | 47 |
| C1127178 | 14 | 10101100100110011001 | TCTATGTGTG | 10 | 48 |
| C1127179 | 14 | 01101011011111000000 | ACTCGACGTA | 10 | 49 |
| C1127179 | 14 | 01010110100111010100 | AGACTGTAGA | 10 | 50 |
| C1127179 | 14 | 01010101010101101001 | AGAGAGACTG | 10 | 51 |
| C1127179 | 14 | 01001001101011010011 | ATGTCTAGCG | 10 | 52 |
| C1127179 | 14 | 00100110111011001001 | CACTACTATG | 10 | 53 |
| C1127179 | 14 | 00110100111100111000 | CGATACGCGT | 10 | 54 |
| C1127179 | 14 | 00110011001110010011 | CGCGCGTGCG | 10 | 55 |
| C1127179 | 14 | 00111010011001010110 | CGTCACAGAC | 10 | 56 |
| C1127179 | 14 | 00111001101010101100 | CGTGTCTCTA | 10 | 57 |
| C1127179 | 14 | 11110101001001010010 | TACGAGCAGC | 10 | 58 |
| C1127179 | 14 | 11010010010010111001 | TAGCATCGTG | 10 | 59 |
| C1127179 | 14 | 11001110011010100100 | TATACACTCA | 10 | 60 |
| C1127179 | 14 | 10101001100110010110 | TCTGTGTGAC | 10 | 61 |
| C1127179 | 14 | 10011101111001001000 | TGTAGTACAT | 10 | 62 |
| C1127180 | 14 | 01101011010010101010 | ACTCGATCTC | 10 | 63 |
| C1127180 | 14 | 01010110100111010100 | AGACTGTAGA | 10 | 64 |
| C1127180 | 14 | 01010101010101101001 | AGAGAGACTG | 10 | 65 |
| C1127180 | 14 | 01001001101011010011 | ATGTCTAGCG | 10 | 66 |
| C1127180 | 14 | 00100110111011001001 | CACTACTATG | 10 | 67 |
| C1127180 | 14 | 00110100111100111000 | CGATACGCGT | 10 | 68 |
| C1127180 | 14 | 00110011001110010011 | CGCGCGTGCG | 10 | 69 |
| C1127180 | 14 | 00111010011001010110 | CGTCACAGAC | 10 | 70 |
| C1127180 | 14 | 00111001101010101100 | CGTGTCTCTA | 10 | 71 |
| C1127180 | 14 | 11110101001001010010 | TACGAGCAGC | 10 | 72 |
| C1127180 | 14 | 11010010010010111001 | TAGCATCGTG | 10 | 73 |
| C1127180 | 14 | 11001110011010100100 | TATACACTCA | 10 | 74 |
| C1127180 | 14 | 10101001100110010110 | TCTGTGTGAC | 10 | 75 |
| C1127180 | 14 | 10011101111001001000 | TGTAGTACAT | 10 | 76 |
| C1127181 | 14 | 01100110011100101001 | ACACACGCTG | 10 | 77 |
| C1127181 | 14 | 01110100101001001101 | ACGATCATAG | 10 | 78 |
| C1127181 | 14 | 01010101010101100110 | AGAGAGACAC | 10 | 79 |
| C1127181 | 14 | 01001110110010010110 | ATACTATGAC | 10 | 80 |
| C1127181 | 14 | 00110011001110010011 | CGCGCGTGCG | 10 | 81 |
| C1127181 | 14 | 00111001101010101100 | CGTGTCTCTA | 10 | 82 |
| C1127181 | 14 | 00101111011001011000 | CTACGACAGT | 10 | 83 |
| C1127181 | 14 | 00101001110101001011 | CTGTAGATCG | 10 | 84 |
| C1127181 | 14 | 11010010010110101100 | TAGCAGTCTA | 10 | 85 |
| C1127181 | 14 | 11011001001100111000 | TAGTGCGCGT | 10 | 86 |
| C1127181 | 14 | 10101100100110011001 | TCTATGTGTG | 10 | 87 |
| C1127181 | 14 | 10101011001010100110 | TCTCGCTCAC | 10 | 88 |
| C1127181 | 14 | 10010100111011101000 | TGATACTACT | 10 | 89 |
| C1127181 | 14 | 10011010110101010100 | TGTCTAGAGA | 10 | 90 |
| C1127182 | 14 | 01100101101011110000 | ACAGTCTACG | 10 | 91 |
| C1127182 | 14 | 01010111001001101010 | AGACGCACTC | 10 | 92 |
| C1127182 | 14 | 01010010111001001101 | AGCTACATAG | 10 | 93 |
| C1127182 | 14 | 01011010100110010110 | AGTCTGTGAC | 10 | 94 |
| C1127182 | 14 | 01001101010110011100 | ATAGAGTGTA | 10 | 95 |
| C1127182 | 14 | 00110011001110010011 | CGCGCGTGCG | 10 | 96 |
| C1127182 | 14 | 00111001101010101100 | CGTGTCTCTA | 10 | 97 |
| C1127182 | 14 | 00101110110100101001 | CTACTAGCTG | 10 | 98 |
| C1127182 | 14 | 00101001010101110101 | CTGAGACGAG | 10 | 99 |
| C1127182 | 14 | 11011001001100111000 | TAGTGCGCGT | 10 | 100 |
| C1127182 | 14 | 10100111110010010100 | TCACGTATGA | 10 | 101 |
| C1127182 | 14 | 10111010010101001010 | TCGTCAGATC | 10 | 102 |
| C1127182 | 14 | 10101100111001100100 | TCTATACACA | 10 | 103 |
| C1127182 | 14 | 10010100110110110010 | TGATAGTCGC | 10 | 104 |
| C1127183 | 14 | 01110100101100111000 | ACGATCGCGT | 10 | 105 |
| C1127183 | 14 | 01101010110010011001 | ACTCTATGTG | 10 | 106 |
| C1127183 | 14 | 01010010011001101101 | AGCACACTAG | 10 | 107 |
| C1127183 | 14 | 01001110010101011010 | ATACAGAGTC | 10 | 108 |
| C1127183 | 14 | 01001100101010100111 | ATATCTCACG | 10 | 109 |
| C1127183 | 14 | 00100101110011110010 | CAGTATACGC | 10 | 110 |
| C1127183 | 14 | 00110011001110010011 | CGCGCGTGCG | 10 | 111 |
| C1127183 | 14 | 00111001101010101100 | CGTGTCTCTA | 10 | 112 |
| C1127183 | 14 | 00101111111001001000 | CTACGTACAT | 10 | 113 |
| C1127183 | 14 | 11001111001010010100 | TATACGCTGA | 10 | 114 |
| C1127183 | 14 | 10110110010010101010 | TCGACATCTC | 10 | 115 |
| C1127183 | 14 | 10110010110101100100 | TCGCTAGACA | 10 | 116 |
| C1127183 | 14 | 10010101100100110110 | TGAGTGCGAC | 10 | 117 |
| C1127183 | 14 | 10011001010111011000 | TGTGAGTAGT | 10 | 118 |
| C1127184 | 14 | 01100111001010100110 | ACACGCTCAC | 10 | 119 |
| C1127184 | 14 | 01110100101100111000 | ACGATCGCGT | 10 | 120 |
| C1127184 | 14 | 01010111010101010100 | AGACGAGAGA | 10 | 121 |
| C1127184 | 14 | 01010010100111001110 | AGCTGTATAC | 10 | 122 |
| C1127184 | 14 | 01001101100101001011 | ATAGTGATCG | 10 | 123 |
| C1127184 | 14 | 00100110111001101001 | CACTACACTG | 10 | 124 |
| C1127184 | 14 | 00110011001110010011 | CGCGCGTGCG | 10 | 125 |
| C1127184 | 14 | 00111101011101100000 | CGTAGACGAC | 10 | 126 |
| C1127184 | 14 | 00111001101010101100 | CGTGTCTCTA | 10 | 127 |
| C1127184 | 14 | 11100100110010010101 | TACATATGAG | 10 | 128 |
| C1127184 | 14 | 10101010101101100100 | TCTCTCGACA | 10 | 129 |
| C1127184 | 14 | 10101001010100101101 | TCTGAGCTAG | 10 | 130 |
| C1127184 | 14 | 10010101010011101010 | TGAGATACTC | 10 | 131 |
| C1127184 | 14 | 10011110100110011000 | TGTACTGTGT | 10 | 132 |
| C1127185 | 14 | 01100100101110101001 | ACATCGTCTG | 10 | 133 |
| C1127185 | 14 | 01110010100111011000 | ACGCTGTAGT | 10 | 134 |
| C1127185 | 14 | 01010101010101100110 | AGAGAGACAC | 10 | 135 |
| C1127185 | 14 | 01011010010100111100 | AGTCAGCGTA | 10 | 136 |
| C1127185 | 14 | 01001111001001110100 | ATACGCACGA | 10 | 137 |
| C1127185 | 14 | 00100100100111010111 | CATGTAGACG | 10 | 138 |
| C1127185 | 14 | 00110011001110010011 | CGCGCGTGCG | 10 | 139 |
| C1127185 | 14 | 00111001101010101100 | CGTGTCTCTA | 10 | 140 |
| C1127185 | 14 | 00101110010010011110 | CTACATGTAC | 10 | 141 |
| C1127185 | 14 | 11101110100100101000 | TACTACTGCT | 10 | 142 |
| C1127185 | 14 | 11010101010010011001 | TAGAGATGTG | 10 | 143 |
| C1127185 | 14 | 10100101011011010100 | TCAGACTAGA | 10 | 144 |
| C1127185 | 14 | 10011100101101010010 | TGTATCGAGC | 10 | 145 |
| C1127185 | 14 | 10011011111001001000 | TGTCGTACAT | 10 | 146 |
| C1127186 | 14 | 01100100101110101001 | ACATCGTCTG | 10 | 147 |
| C1127186 | 14 | 01110010100111011000 | ACGCTGTAGT | 10 | 148 |
| C1127186 | 14 | 01010101010101100110 | AGAGAGACAC | 10 | 149 |
| C1127186 | 14 | 01011010010100111100 | AGTCAGCGTA | 10 | 150 |
| C1127186 | 14 | 01001111001001110100 | ATACGCACGA | 10 | 151 |
| C1127186 | 14 | 00100100100111010111 | CATGTAGACG | 10 | 152 |
| C1127186 | 14 | 00110011001110010011 | CGCGCGTGCG | 10 | 153 |
| C1127186 | 14 | 00111001101010101100 | CGTGTCTCTA | 10 | 154 |
| C1127186 | 14 | 00101110010010011110 | CTACATGTAC | 10 | 155 |
| C1127186 | 14 | 11101110100100101000 | TACTACTGCT | 10 | 156 |
| C1127186 | 14 | 11010101010010011001 | TAGAGATGTG | 10 | 157 |
| C1127186 | 14 | 10100101011011010100 | TCAGACTAGA | 10 | 158 |
| C1127186 | 14 | 10110010011001101010 | TCGCACACTC | 10 | 159 |
| C1127186 | 14 | 10011100101101010010 | TGTATCGAGC | 10 | 160 |
| C1127187 | 14 | 01100111001010100110 | ACACGCTCAC | 10 | 161 |
| C1127187 | 14 | 01110010100111011000 | ACGCTGTAGT | 10 | 162 |
| C1127187 | 14 | 01011010010010111010 | AGTCATCGTC | 10 | 163 |
| C1127187 | 14 | 01011001010101100101 | AGTGAGACAG | 10 | 164 |
| C1127187 | 14 | 01001101010110011100 | ATAGAGTGTA | 10 | 165 |
| C1127187 | 14 | 00100110010011110101 | CACATACGAG | 10 | 166 |
| C1127187 | 14 | 00110011001110010011 | CGCGCGTGCG | 10 | 167 |
| C1127187 | 14 | 00111001101010101100 | CGTGTCTCTA | 10 | 168 |
| C1127187 | 14 | 00101010110101101010 | CTCTAGACTC | 10 | 169 |
| C1127187 | 14 | 11001110101001010100 | TATACTCAGA | 10 | 170 |
| C1127187 | 14 | 11001011110010110000 | TATCGTATCG | 10 | 171 |
| C1127187 | 14 | 10111110010011001000 | TCGTACATAT | 10 | 172 |
| C1127187 | 14 | 10101001100110010110 | TCTGTGTGAC | 10 | 173 |
| C1127187 | 14 | 10010111011100101000 | TGACGACGCT | 10 | 174 |
| C1127188 | 14 | 01100100101110101001 | ACATCGTCTG | 10 | 175 |
| C1127188 | 14 | 01110010011101001100 | ACGCACGATA | 10 | 176 |
| C1127188 | 14 | 01010101110100111000 | AGAGTAGCGT | 10 | 177 |
| C1127188 | 14 | 01011110011001101000 | AGTACACACT | 10 | 178 |
| C1127188 | 14 | 01011010100110010110 | AGTCTGTGAC | 10 | 179 |
| C1127188 | 14 | 00100110111011010010 | CACTACTAGC | 10 | 180 |
| C1127188 | 14 | 00110011001110010011 | CGCGCGTGCG | 10 | 181 |
| C1127188 | 14 | 00111001101010101100 | CGTGTCTCTA | 10 | 182 |
| C1127188 | 14 | 00101110010110011100 | CTACAGTGTA | 10 | 183 |
| C1127188 | 14 | 00101001110101001011 | CTGTAGATCG | 10 | 184 |
| C1127188 | 14 | 11001011001101011000 | TATCGCGAGT | 10 | 185 |
| C1127188 | 14 | 10110110010100100101 | TCGACAGCAG | 10 | 186 |
| C1127188 | 14 | 10101010010011110100 | TCTCATACGA | 10 | 187 |
| C1127188 | 14 | 10010101010011001110 | TGAGATATAC | 10 | 188 |
| C1127189 | 14 | 01100101001010110110 | ACAGCTCGAC | 10 | 189 |
| C1127189 | 14 | 01101011010011100100 | ACTCGATACA | 10 | 190 |
| C1127189 | 14 | 01010100110101101100 | AGATAGACTA | 10 | 191 |
| C1127189 | 14 | 01010011001110011001 | AGCGCGTGTG | 10 | 192 |
| C1127189 | 14 | 01001001101011010011 | ATGTCTAGCG | 10 | 193 |
| C1127189 | 14 | 00100111110111001000 | CACGTAGTAT | 10 | 194 |
| C1127189 | 14 | 00110110011100100101 | CGACACGCAG | 10 | 195 |
| C1127189 | 14 | 00111001010101010110 | CGTGAGAGAC | 10 | 196 |
| C1127189 | 14 | 00111001101010101100 | CGTGTCTCTA | 10 | 197 |
| C1127189 | 14 | 11101100101100101000 | TACTATCGCT | 10 | 198 |
| C1127189 | 14 | 11011001001001100101 | TAGTGCACAG | 10 | 199 |
| C1127189 | 14 | 10110010010101111000 | TCGCAGACGT | 10 | 200 |
| C1127189 | 14 | 10101110100110010100 | TCTACTGTGA | 10 | 201 |
| C1127189 | 14 | 10010111101001001010 | TGACGTCATC | 10 | 202 |
| C1127190 | 14 | 01100101011001001101 | ACAGACATAG | 10 | 203 |
| C1127190 | 14 | 01110011001110011000 | ACGCGCGTGT | 10 | 204 |
| C1127190 | 14 | 01010111001001101010 | AGACGCACTC | 10 | 205 |
| C1127190 | 14 | 01010010110010110101 | AGCTATCGAG | 10 | 206 |
| C1127190 | 14 | 01001100100110011110 | ATATGTGTAC | 10 | 207 |
| C1127190 | 14 | 01001001110101111000 | ATGTAGACGT | 10 | 208 |
| C1127190 | 14 | 00100110101110100110 | CACTCGTCAC | 10 | 209 |
| C1127190 | 14 | 00111001010101010110 | CGTGAGAGAC | 10 | 210 |
| C1127190 | 14 | 00111001101010101100 | CGTGTCTCTA | 10 | 211 |
| C1127190 | 14 | 00101010011011011001 | CTCACTAGTG | 10 | 212 |
| C1127190 | 14 | 11101100110010101000 | TACTATATCT | 10 | 213 |
| C1127190 | 14 | 11001110101001010100 | TATACTCAGA | 10 | 214 |
| C1127190 | 14 | 10110010010111100100 | TCGCAGTACA | 10 | 215 |
| C1127190 | 14 | 10010101100100110011 | TGAGTGCGCG | 10 | 216 |
| C1127191 | 14 | 01100101011010011010 | ACAGACTGTC | 10 | 217 |
| C1127191 | 14 | 01010011001111100100 | AGCGCGTACA | 10 | 218 |
| C1127191 | 14 | 01011011010100101001 | AGTCGAGCTG | 10 | 219 |
| C1127191 | 14 | 01001110110101010010 | ATACTAGAGC | 10 | 220 |
| C1127191 | 14 | 01001011101001111000 | ATCGTCACGT | 10 | 221 |
| C1127191 | 14 | 00110101001001110011 | CGAGCACGCG | 10 | 222 |
| C1127191 | 14 | 00111001010101010110 | CGTGAGAGAC | 10 | 223 |
| C1127191 | 14 | 00111001101010101100 | CGTGTCTCTA | 10 | 224 |
| C1127191 | 14 | 00101010010010111101 | CTCATCGTAG | 10 | 225 |
| C1127191 | 14 | 11100110101100101000 | TACACTCGCT | 10 | 226 |
| C1127191 | 14 | 11001001110111001000 | TATGTAGTAT | 10 | 227 |
| C1127191 | 14 | 10110010100110011001 | TCGCTGTGTG | 10 | 228 |
| C1127191 | 14 | 10101100111001100100 | TCTATACACA | 10 | 229 |
| C1127191 | 14 | 10010111101001001010 | TGACGTCATC | 10 | 230 |
| C1127192 | 14 | 01101001100101001011 | ACTGTGATCG | 10 | 231 |
| C1127192 | 14 | 01010011001111100100 | AGCGCGTACA | 10 | 232 |
| C1127192 | 14 | 01011110100110011000 | AGTACTGTGT | 10 | 233 |
| C1127192 | 14 | 01001110101001100110 | ATACTCACAC | 10 | 234 |
| C1127192 | 14 | 00100110010111011100 | CACAGTAGTA | 10 | 235 |
| C1127192 | 14 | 00110101001001110011 | CGAGCACGCG | 10 | 236 |
| C1127192 | 14 | 00111001010101010110 | CGTGAGAGAC | 10 | 237 |
| C1127192 | 14 | 00111001101010101100 | CGTGTCTCTA | 10 | 238 |
| C1127192 | 14 | 00101010010010101111 | CTCATCTACG | 10 | 239 |
| C1127192 | 14 | 10110010110010010101 | TCGCTATGAG | 10 | 240 |
| C1127192 | 14 | 10101100111001001100 | TCTATACATA | 10 | 241 |
| C1127192 | 14 | 10101011001100111000 | TCTCGCGCGT | 10 | 242 |
| C1127192 | 14 | 10010111011011001000 | TGACGACTAT | 10 | 243 |
| C1127192 | 14 | 10010100110110110010 | TGATAGTCGC | 10 | 244 |
| C1127193 | 14 | 01101001100101001011 | ACTGTGATCG | 10 | 245 |
| C1127193 | 14 | 01010011001111100100 | AGCGCGTACA | 10 | 246 |
| C1127193 | 14 | 01011110100110011000 | AGTACTGTGT | 10 | 247 |
| C1127193 | 14 | 00100110010111011100 | CACAGTAGTA | 10 | 248 |
| C1127193 | 14 | 00110101001001110011 | CGAGCACGCG | 10 | 249 |
| C1127193 | 14 | 00111001010101010110 | CGTGAGAGAC | 10 | 250 |
| C1127193 | 14 | 00111001101010101100 | CGTGTCTCTA | 10 | 251 |
| C1127193 | 14 | 00101010010010101111 | CTCATCTACG | 10 | 252 |
| C1127193 | 14 | 11010010101001101010 | TAGCTCACTC | 10 | 253 |
| C1127193 | 14 | 10110010110010010101 | TCGCTATGAG | 10 | 254 |
| C1127193 | 14 | 10101100111001001100 | TCTATACATA | 10 | 255 |
| C1127193 | 14 | 10101011001100111000 | TCTCGCGCGT | 10 | 256 |
| C1127193 | 14 | 10010111011011001000 | TGACGACTAT | 10 | 257 |
| C1127193 | 14 | 10010100110110110010 | TGATAGTCGC | 10 | 258 |
| C1127194 | 14 | 01101001100101001011 | ACTGTGATCG | 10 | 259 |
| C1127194 | 14 | 01010011001111100100 | AGCGCGTACA | 10 | 260 |
| C1127194 | 14 | 01011100111001011000 | AGTATACAGT | 10 | 261 |
| C1127194 | 14 | 00100100111110011001 | CATACGTGTG | 10 | 262 |
| C1127194 | 14 | 00110101001001110011 | CGAGCACGCG | 10 | 263 |
| C1127194 | 14 | 00111001010101010110 | CGTGAGAGAC | 10 | 264 |
| C1127194 | 14 | 00111001101010101100 | CGTGTCTCTA | 10 | 265 |
| C1127194 | 14 | 00101010010010101111 | CTCATCTACG | 10 | 266 |
| C1127194 | 14 | 11100100110101001100 | TACATAGATA | 10 | 267 |
| C1127194 | 14 | 11010010101001101010 | TAGCTCACTC | 10 | 268 |
| C1127194 | 14 | 10110010110010010101 | TCGCTATGAG | 10 | 269 |
| C1127194 | 14 | 10101011001100111000 | TCTCGCGCGT | 10 | 270 |
| C1127194 | 14 | 10010111011011001000 | TGACGACTAT | 10 | 271 |
| C1127194 | 14 | 10010100110110110010 | TGATAGTCGC | 10 | 272 |
| C1127195 | 14 | 01101110101001010100 | ACTACTCAGA | 10 | 273 |
| C1127195 | 14 | 01101001100101001011 | ACTGTGATCG | 10 | 274 |
| C1127195 | 14 | 01010011001111100100 | AGCGCGTACA | 10 | 275 |
| C1127195 | 14 | 00100100111110011001 | CATACGTGTG | 10 | 276 |
| C1127195 | 14 | 00110101001001110011 | CGAGCACGCG | 10 | 277 |
| C1127195 | 14 | 00111001010101010110 | CGTGAGAGAC | 10 | 278 |
| C1127195 | 14 | 00111001101010101100 | CGTGTCTCTA | 10 | 279 |
| C1127195 | 14 | 00101010010010101111 | CTCATCTACG | 10 | 280 |
| C1127195 | 14 | 11100100110101001100 | TACATAGATA | 10 | 281 |
| C1127195 | 14 | 11010010101001101010 | TAGCTCACTC | 10 | 282 |
| C1127195 | 14 | 10110010110010010101 | TCGCTATGAG | 10 | 283 |
| C1127195 | 14 | 10101011001100111000 | TCTCGCGCGT | 10 | 284 |
| C1127195 | 14 | 10010111011011001000 | TGACGACTAT | 10 | 285 |
| C1127195 | 14 | 10010100110110110010 | TGATAGTCGC | 10 | 286 |
| C1127196 | 14 | 01100101011010011010 | ACAGACTGTC | 10 | 287 |
| C1127196 | 14 | 01101011001100101001 | ACTCGCGCTG | 10 | 288 |
| C1127196 | 14 | 01010011001111100100 | AGCGCGTACA | 10 | 289 |
| C1127196 | 14 | 01011100111001001001 | AGTATACATG | 10 | 290 |
| C1127196 | 14 | 01001110010110110100 | ATACAGTCGA | 10 | 291 |
| C1127196 | 14 | 00100111110111001000 | CACGTAGTAT | 10 | 292 |
| C1127196 | 14 | 00110101001001110011 | CGAGCACGCG | 10 | 293 |
| C1127196 | 14 | 00111001010101010110 | CGTGAGAGAC | 10 | 294 |
| C1127196 | 14 | 00111001101010101100 | CGTGTCTCTA | 10 | 295 |
| C1127196 | 14 | 00101010010010101111 | CTCATCTACG | 10 | 296 |
| C1127196 | 14 | 11010010101001101010 | TAGCTCACTC | 10 | 297 |
| C1127196 | 14 | 11001001100110010011 | TATGTGTGCG | 10 | 298 |
| C1127196 | 14 | 10110100101110010100 | TCGATCGTGA | 10 | 299 |
| C1127196 | 14 | 100101i0100101001101 | TGACTGATAG | 10 | 300 |
| C1127197 | 14 | 01100101011001001101 | ACAGACATAG | 10 | 301 |
| C1127197 | 14 | 01101011001100101001 | ACTCGCGCTG | 10 | 302 |
| C1127197 | 14 | 01010011001111100100 | AGCGCGTACA | 10 | 303 |
| C1127197 | 14 | 01011100111001011000 | AGTATACAGT | 10 | 304 |
| C1127197 | 14 | 01001110010110110100 | ATACAGTCGA | 10 | 305 |
| C1127197 | 14 | 00100111110111001000 | CACGTAGTAT | 10 | 306 |
| C1127197 | 14 | 00110101001001110011 | CGAGCACGCG | 10 | 307 |
| C1127197 | 14 | 00111001010101010110 | CGTGAGAGAC | 10 | 308 |
| C1127197 | 14 | 00111001101010101100 | CGTGTCTCTA | 10 | 309 |
| C1127197 | 14 | 00101010010010101111 | CTCATCTACG | 10 | 310 |
| C1127197 | 14 | 11010100110010101010 | TAGATATCTC | 10 | 311 |
| C1127197 | 14 | 11001001100110010011 | TATGTGTGCG | 10 | 312 |
| C1127197 | 14 | 10110100101110010100 | TCGATCGTGA | 10 | 313 |
| C1127197 | 14 | 10011110100101001001 | TGTACTGATG | 10 | 314 |
| C1127198 | 14 | 01100101011001001101 | ACAGACATAG | 10 | 315 |
| C1127198 | 14 | 01101011001100101001 | ACTCGCGCTG | 10 | 316 |
| C1127198 | 14 | 01010011001111100100 | AGCGCGTACA | 10 | 317 |
| C1127198 | 14 | 01011100111001011000 | AGTATACAGT | 10 | 318 |
| C1127198 | 14 | 01001110010110110100 | ATACAGTCGA | 10 | 319 |
| C1127198 | 14 | 00100111110111001000 | CACGTAGTAT | 10 | 320 |
| C1127198 | 14 | 00110101001001110011 | CGAGCACGCG | 10 | 321 |
| C1127198 | 14 | 00111001010101010110 | CGTGAGAGAC | 10 | 322 |
| C1127198 | 14 | 00111001101010101100 | CGTGTCTCTA | 10 | 323 |
| C1127198 | 14 | 00101010010010101111 | CTCATCTACG | 10 | 324 |
| C1127198 | 14 | 11010010110100101010 | TAGCTAGCTC | 10 | 325 |
| C1127198 | 14 | 11001001100110010011 | TATGTGTGCG | 10 | 326 |
| C1127198 | 14 | 10110100101110010100 | TCGATCGTGA | 10 | 327 |
| C1127198 | 14 | 10011110100101001001 | TGTACTGATG | 10 | 328 |
| C1127199 | 14 | 01100101011001001101 | ACAGACATAG | 10 | 329 |
| C1127199 | 14 | 01101011001100101001 | ACTCGCGCTG | 10 | 330 |
| C1127199 | 14 | 01010011001111100100 | AGCGCGTACA | 10 | 331 |
| C1127199 | 14 | 01011100111001011000 | AGTATACAGT | 10 | 332 |
| C1127199 | 14 | 01001110010110110100 | ATACAGTCGA | 10 | 333 |
| C1127199 | 14 | 00100111110111001000 | CACGTAGTAT | 10 | 334 |
| C1127199 | 14 | 00110101001001110011 | CGAGCACGCG | 10 | 335 |
| C1127199 | 14 | 00111001010101010110 | CGTGAGAGAC | 10 | 336 |
| C1127199 | 14 | 00111001101010101100 | CGTGTCTCTA | 10 | 337 |
| C1127199 | 14 | 00101010010010101111 | CTCATCTACG | 10 | 338 |
| C1127199 | 14 | 11010010101001101010 | TAGCTCACTC | 10 | 339 |
| C1127199 | 14 | 11001001100110010011 | TATGTGTGCG | 10 | 340 |
| C1127199 | 14 | 10110100101110010100 | TCGATCGTGA | 10 | 341 |
| C1127199 | 14 | 10011110100101001001 | TGTACTGATG | 10 | 342 |
| C1127200 | 14 | 01100101011001001101 | ACAGACATAG | 10 | 343 |
| C1127200 | 14 | 01101011001100101001 | ACTCGCGCTG | 10 | 344 |
| C1127200 | 14 | 01010011001111100100 | AGCGCGTACA | 10 | 345 |
| C1127200 | 14 | 01001110010110110100 | ATACAGTCGA | 10 | 346 |
| C1127200 | 14 | 00100111110111001000 | CACGTAGTAT | 10 | 347 |
| C1127200 | 14 | 00110101001001110011 | CGAGCACGCG | 10 | 348 |
| C1127200 | 14 | 00111001010101010110 | CGTGAGAGAC | 10 | 349 |
| C1127200 | 14 | 00111001101010101100 | CGTGTCTCTA | 10 | 350 |
| C1127200 | 14 | 00101010010010101111 | CTCATCTACG | 10 | 351 |
| C1127200 | 14 | 11010100110010101010 | TAGATATCTC | 10 | 352 |
| C1127200 | 14 | 11010011011001011000 | TAGCGACAGT | 10 | 353 |
| C1127200 | 14 | 11001001100110010011 | TATGTGTGCG | 10 | 354 |
| C1127200 | 14 | 10110100101110010100 | TCGATCGTGA | 10 | 355 |
| C1127200 | 14 | 10011110100101001001 | TGTACTGATG | 10 | 356 |
| C1127201 | 14 | 01101011001100101001 | ACTCGCGCTG | 10 | 357 |
| C1127201 | 14 | 01010011001111100100 | AGCGCGTACA | 10 | 358 |
| C1127201 | 14 | 01001110010110110100 | ATACAGTCGA | 10 | 359 |
| C1127201 | 14 | 01001010111001100110 | ATCTACACAC | 10 | 360 |
| C1127201 | 14 | 00100111110111001000 | CACGTAGTAT | 10 | 361 |
| C1127201 | 14 | 00110101001001110011 | CGAGCACGCG | 10 | 362 |
| C1127201 | 14 | 00111001010101010110 | CGTGAGAGAC | 10 | 363 |
| C1127201 | 14 | 00111001101010101100 | CGTGTCTCTA | 10 | 364 |
| C1127201 | 14 | 00101010010010101111 | CTCATCTACG | 10 | 365 |
| C1127201 | 14 | 11010100110010101010 | TAGATATCTC | 10 | 366 |
| C1127201 | 14 | 11010011011001011000 | TAGCGACAGT | 10 | 367 |
| C1127201 | 14 | 11001001100110010011 | TATGTGTGCG | 10 | 368 |
| C1127201 | 14 | 10110100101110010100 | TCGATCGTGA | 10 | 369 |
| C1127201 | 14 | 10011110100101001001 | TGTACTGATG | 10 | 370 |
| C1127202 | 14 | 01100101011001001101 | ACAGACATAG | 10 | 371 |
| C1127202 | 14 | 01101011001100101001 | ACTCGCGCTG | 10 | 372 |
| C1127202 | 14 | 01101010110010010110 | ACTCTATGAC | 10 | 373 |
| C1127202 | 14 | 01010011001111100100 | AGCGCGTACA | 10 | 374 |
| C1127202 | 14 | 01011100111001011000 | AGTATACAGT | 10 | 375 |
| C1127202 | 14 | 01001110010110110100 | ATACAGTCGA | 10 | 376 |
| C1127202 | 14 | 00100111110111001000 | CACGTAGTAT | 10 | 377 |
| C1127202 | 14 | 00110101001001110011 | CGAGCACGCG | 10 | 378 |
| C1127202 | 14 | 00111001010101010110 | CGTGAGAGAC | 10 | 379 |
| C1127202 | 14 | 00111001101010101100 | CGTGTCTCTA | 10 | 380 |
| C1127202 | 14 | 11010100110010101010 | TAGATATCTC | 10 | 381 |
| C1127202 | 14 | 11001001100110010011 | TATGTGTGCG | 10 | 382 |
| C1127202 | 14 | 10110100101110010100 | TCGATCGTGA | 10 | 383 |
| C1127202 | 14 | 10011110100101001001 | TGTACTGATG | 10 | 384 |
| C1127203 | 14 | 01100101011001001101 | ACAGACATAG | 10 | 385 |
| C1127203 | 14 | 01101011001100101001 | ACTCGCGCTG | 10 | 386 |
| C1127203 | 14 | 01101010110010010110 | ACTCTATGAC | 10 | 387 |
| C1127203 | 14 | 01010011001111100100 | AGCGCGTACA | 10 | 388 |
| C1127203 | 14 | 01011100111001011000 | AGTATACAGT | 10 | 389 |
| C1127203 | 14 | 01001110010110110100 | ATACAGTCGA | 10 | 390 |
| C1127203 | 14 | 00100111110111001000 | CACGTAGTAT | 10 | 391 |
| C1127203 | 14 | 00110101001001110011 | CGAGCACGCG | 10 | 392 |
| C1127203 | 14 | 00111001010101010110 | CGTGAGAGAC | 10 | 393 |
| C1127203 | 14 | 00111001101010101100 | CGTGTCTCTA | 10 | 394 |
| C1127203 | 14 | 11010010110100101010 | TAGCTAGCTC | 10 | 395 |
| C1127203 | 14 | 11001001100110010011 | TATGTGTGCG | 10 | 396 |
| C1127203 | 14 | 10110100101110010100 | TCGATCGTGA | 10 | 397 |
| C1127203 | 14 | 10011110100101001001 | TGTACTGATG | 10 | 398 |
| C1127204 | 14 | 01100101011001001101 | ACAGACATAG | 10 | 399 |
| C1127204 | 14 | 01101011001100101001 | ACTCGCGCTG | 10 | 400 |
| C1127204 | 14 | 01101010110010010110 | ACTCTATGAC | 10 | 401 |
| C1127204 | 14 | 01010011001111100100 | AGCGCGTACA | 10 | 402 |
| C1127204 | 14 | 01011100111001011000 | AGTATACAGT | 10 | 403 |
| C1127204 | 14 | 01001110010110110100 | ATACAGTCGA | 10 | 404 |
| C1127204 | 14 | 00100111110111001000 | CACGTAGTAT | 10 | 405 |
| C1127204 | 14 | 00110101001001110011 | CGAGCACGCG | 10 | 406 |
| C1127204 | 14 | 00111001010101010110 | CGTGAGAGAC | 10 | 407 |
| C1127204 | 14 | 00111001101010101100 | CGTGTCTCTA | 10 | 408 |
| C1127204 | 14 | 11010010101001101010 | TAGCTCACTC | 10 | 409 |
| C1127204 | 14 | 11001001100110010011 | TATGTGTGCG | 10 | 410 |
| C1127204 | 14 | 10110100101110010100 | TCGATCGTGA | 10 | 411 |
| C1127204 | 14 | 10011110100101001001 | TGTACTGATG | 10 | 412 |
| C1127205 | 14 | 01100101011001001101 | ACAGACATAG | 10 | 413 |
| C1127205 | 14 | 01101011001100101001 | ACTCGCGCTG | 10 | 414 |
| C1127205 | 14 | 01101010110010010110 | ACTCTATGAC | 10 | 415 |
| C1127205 | 14 | 01010011001111100100 | AGCGCGTACA | 10 | 416 |
| C1127205 | 14 | 01001110010110110100 | ATACAGTCGA | 10 | 417 |
| C1127205 | 14 | 00100111110111001000 | CACGTAGTAT | 10 | 418 |
| C1127205 | 14 | 00110101001001110011 | CGAGCACGCG | 10 | 419 |
| C1127205 | 14 | 00111001010101010110 | CGTGAGAGAC | 10 | 420 |
| C1127205 | 14 | 00111001101010101100 | CGTGTCTCTA | 10 | 421 |
| C1127205 | 14 | 11010100110010101010 | TAGATATCTC | 10 | 422 |
| C1127205 | 14 | 11001011011001011000 | TATCGACAGT | 10 | 423 |
| C1127205 | 14 | 11001001100110010011 | TATGTGTGCG | 10 | 424 |
| C1127205 | 14 | 10110100101110010100 | TCGATCGTGA | 10 | 425 |
| C1127205 | 14 | 10011110100101001001 | TGTACTGATG | 10 | 426 |
| C1127206 | 14 | 01100101011001001101 | ACAGACATAG | 10 | 427 |
| C1127206 | 14 | 01101011001100101001 | ACTCGCGCTG | 10 | 428 |
| C1127206 | 14 | 01101010110010010110 | ACTCTATGAC | 10 | 429 |
| C1127206 | 14 | 01010011001111100100 | AGCGCGTACA | 10 | 430 |
| C1127206 | 14 | 01001110010110110100 | ATACAGTCGA | 10 | 431 |
| C1127206 | 14 | 00100111110111001000 | CACGTAGTAT | 10 | 432 |
| C1127206 | 14 | 00110101001001110011 | CGAGCACGCG | 10 | 433 |
| C1127206 | 14 | 00111001010101010110 | CGTGAGAGAC | 10 | 434 |
| C1127206 | 14 | 00111001101010101100 | CGTGTCTCTA | 10 | 435 |
| C1127206 | 14 | 11010010110100101010 | TAGCTAGCTC | 10 | 436 |
| C1127206 | 14 | 11001011011001011000 | TATCGACAGT | 10 | 437 |
| C1127206 | 14 | 11001001100110010011 | TATGTGTGCG | 10 | 438 |
| C1127206 | 14 | 10110100101110010100 | TCGATCGTGA | 10 | 439 |
| C1127206 | 14 | 10011110100101001001 | TGTACTGATG | 10 | 440 |
| C1127207 | 14 | 01100101011001001101 | ACAGACATAG | 10 | 441 |
| C1127207 | 14 | 01101011001100101001 | ACTCGCGCTG | 10 | 442 |
| C1127207 | 14 | 01101010110010010110 | ACTCTATGAC | 10 | 443 |
| C1127207 | 14 | 01010011001111100100 | AGCGCGTACA | 10 | 444 |
| C1127207 | 14 | 01001110010110110100 | ATACAGTCGA | 10 | 445 |
| C1127207 | 14 | 00100111110111001000 | CACGTAGTAT | 10 | 446 |
| C1127207 | 14 | 00110101001001110011 | CGAGCACGCG | 10 | 447 |
| C1127207 | 14 | 00111001010101010110 | CGTGAGAGAC | 10 | 448 |
| C1127207 | 14 | 00111001101010101100 | CGTGTCTCTA | 10 | 449 |
| C1127207 | 14 | 11010100110010101010 | TAGATATCTC | 10 | 450 |
| C1127207 | 14 | 11010011011001011000 | TAGCGACAGT | 10 | 451 |
| C1127207 | 14 | 11001001100110010011 | TATGTGTGCG | 10 | 452 |
| C1127207 | 14 | 10110100101110010100 | TCGATCGTGA | 10 | 453 |
| C1127207 | 14 | 10011110100101001001 | TGTACTGATG | 10 | 454 |
| C1127208 | 14 | 01100100110011110010 | ACATATACGC | 10 | 455 |
| C1127208 | 14 | 01101011001100101001 | ACTCGCGCTG | 10 | 456 |
| C1127208 | 14 | 01010011001111100100 | AGCGCGTACA | 10 | 457 |
| C1127208 | 14 | 01011110100110011000 | AGTACTGTGT | 10 | 458 |
| C1127208 | 14 | 00100110010111011100 | CACAGTAGTA | 10 | 459 |
| C1127208 | 14 | 00110101001001110011 | CGAGCACGCG | 10 | 460 |
| C1127208 | 14 | 00111001010101010110 | CGTGAGAGAC | 10 | 461 |
| C1127208 | 14 | 00111001101010101100 | CGTGTCTCTA | 10 | 462 |
| C1127208 | 14 | 00101010111001001011 | CTCTACATCG | 10 | 463 |
| C1127208 | 14 | 11010010110100101010 | TAGCTAGCTC | 10 | 464 |
| C1127208 | 14 | 11001011011001011000 | TATCGACAGT | 10 | 465 |
| C1127208 | 14 | 11001001100110010011 | TATGTGTGCG | 10 | 466 |
| C1127208 | 14 | 10110100101110010100 | TCGATCGTGA | 10 | 467 |
| C1127208 | 14 | 10010010011010110110 | TGCACTCGAC | 10 | 468 |
| C1127209 | 14 | 01101011001100101001 | ACTCGCGCTG | 10 | 469 |
| C1127209 | 14 | 01010011001111100100 | AGCGCGTACA | 10 | 470 |
| C1127209 | 14 | 01001101101011011000 | ATAGTCTAGT | 10 | 471 |
| C1127209 | 14 | 01001010010011100111 | ATCATACACG | 10 | 472 |
| C1127209 | 14 | 00100111110111001000 | CACGTAGTAT | 10 | 473 |
| C1127209 | 14 | 00110101001001110011 | CGAGCACGCG | 10 | 474 |
| C1127209 | 14 | 00111001010101010110 | CGTGAGAGAC | 10 | 475 |
| C1127209 | 14 | 00111001101010101100 | CGTGTCTCTA | 10 | 476 |
| C1127209 | 14 | 00101110100101110100 | CTACTGACGA | 10 | 477 |
| C1127209 | 14 | 11010100110010101010 | TAGATATCTC | 10 | 478 |
| C1127209 | 14 | 11001111001001001100 | TATACGCATA | 10 | 479 |
| C1127209 | 14 | 11001001100110010011 | TATGTGTGCG | 10 | 480 |
| C1127209 | 14 | 10110100101110010100 | TCGATCGTGA | 10 | 481 |
| C1127209 | 14 | 10010010011010110110 | TGCACTCGAC | 10 | 482 |
| C1127210 | 14 | 01100101011001001101 | ACAGACATAG | 10 | 483 |
| C1127210 | 14 | 01101011001100101001 | ACTCGCGCTG | 10 | 484 |
| C1127210 | 14 | 01010011001111100100 | AGCGCGTACA | 10 | 485 |
| C1127210 | 14 | 01011100111001011000 | AGTATACAGT | 10 | 486 |
| C1127210 | 14 | 01001110010110110100 | ATACAGTCGA | 10 | 487 |
| C1127210 | 14 | 00100111110111001000 | CACGTAGTAT | 10 | 488 |
| C1127210 | 14 | 00110101001001110011 | CGAGCACGCG | 10 | 489 |
| C1127210 | 14 | 00111001010101010110 | CGTGAGAGAC | 10 | 490 |
| C1127210 | 14 | 00111001101010101100 | CGTGTCTCTA | 10 | 491 |
| C1127210 | 14 | 00101010010010101111 | CTCATCTACG | 10 | 492 |
| C1127210 | 14 | 11010100110010101010 | TAGATATCTC | 10 | 493 |
| C1127210 | 14 | 11001001100110010011 | TATGTGTGCG | 10 | 494 |
| C1127210 | 14 | 10110010101101011000 | TCGCTCGAGT | 10 | 495 |
| C1127210 | 14 | 10101110100100100110 | TCTACTGCAC | 10 | 496 |
| C1127211 | 14 | 01100101011001001101 | ACAGACATAG | 10 | 497 |
| C1127211 | 14 | 01101011001100101001 | ACTCGCGCTG | 10 | 498 |
| C1127211 | 14 | 01010011001111100100 | AGCGCGTACA | 10 | 499 |
| C1127211 | 14 | 01011100111001011000 | AGTATACAGT | 10 | 500 |
| C1127211 | 14 | 01001110010110110100 | ATACAGTCGA | 10 | 501 |
| C1127211 | 14 | 00100111110111001000 | CACGTAGTAT | 10 | 502 |
| C1127211 | 14 | 00110101001001110011 | CGAGCACGCG | 10 | 503 |
| C1127211 | 14 | 00111001010101010110 | CGTGAGAGAC | 10 | 504 |
| C1127211 | 14 | 00111001101010101100 | CGTGTCTCTA | 10 | 505 |
| C1127211 | 14 | 00101010010010101111 | CTCATCTACG | 10 | 506 |
| C1127211 | 14 | 11010100110010101010 | TAGATATCTC | 10 | 507 |
| C1127211 | 14 | 11001001100110010011 | TATGTGTGCG | 10 | 508 |
| C1127211 | 14 | 10110010101101011000 | TCGCTCGAGT | 10 | 509 |
| C1127211 | 14 | 10011110100101001001 | TGTACTGATG | 10 | 510 |
| C1127212 | 14 | 01100101011001001101 | ACAGACATAG | 10 | 511 |
| C1127212 | 14 | 01101011001100101001 | ACTCGCGCTG | 10 | 512 |
| C1127212 | 14 | 01101010110010010110 | ACTCTATGAC | 10 | 513 |
| C1127212 | 14 | 01010011001111100100 | AGCGCGTACA | 10 | 514 |
| C1127212 | 14 | 01011100111001011000 | AGTATACAGT | 10 | 515 |
| C1127212 | 14 | 01001110010110110100 | ATACAGTCGA | 10 | 516 |
| C1127212 | 14 | 00100111110111001000 | CACGTAGTAT | 10 | 517 |
| C1127212 | 14 | 00110101001001110011 | CGAGCACGCG | 10 | 518 |
| C1127212 | 14 | 00111001010101010110 | CGTGAGAGAC | 10 | 519 |
| C1127212 | 14 | 00111001101010101100 | CGTGTCTCTA | 10 | 520 |
| C1127212 | 14 | 11010100110010101010 | TAGATATCTC | 10 | 521 |
| C1127212 | 14 | 11001001100110010011 | TATGTGTGCG | 10 | 522 |
| C1127212 | 14 | 10110010101101011000 | TCGCTCGAGT | 10 | 523 |
| C1127212 | 14 | 10011110100101001001 | TGTACTGATG | 10 | 524 |
| C1127213 | 14 | 01100111011010010010 | ACACGACTGC | 10 | 525 |
| C1127213 | 14 | 01011100111001011000 | AGTATACAGT | 10 | 526 |
| C1127213 | 14 | 01001110101001001011 | ATACTCATCG | 10 | 527 |
| C1127213 | 14 | 01001011010011101100 | ATCGATACTA | 10 | 528 |
| C1127213 | 14 | 00110101001001110011 | CGAGCACGCG | 10 | 529 |
| C1127213 | 14 | 00111001010101010110 | CGTGAGAGAC | 10 | 530 |
| C1127213 | 14 | 00111001101010101100 | CGTGTCTCTA | 10 | 531 |
| C1127213 | 14 | 00101110010110011100 | CTACAGTGTA | 10 | 532 |
| C1127213 | 14 | 00101010111100100110 | CTCTACGCAC | 10 | 533 |
| C1127213 | 14 | 11010010101010010110 | TAGCTCTGAC | 10 | 534 |
| C1127213 | 14 | 10100110010011010101 | TCACATAGAG | 10 | 535 |
| C1127213 | 14 | 10101001100110010011 | TCTGTGTGCG | 10 | 536 |
| C1127213 | 14 | 10010100110110110010 | TGATAGTCGC | 10 | 537 |
| C1127213 | 14 | 10011011001101001001 | TGTCGCGATG | 10 | 538 |
| C1127214 | 14 | 01100110101010011010 | ACACTCTGTC | 10 | 539 |
| C1127214 | 14 | 01011100111001011000 | AGTATACAGT | 10 | 540 |
| C1127214 | 14 | 01001101001110100101 | ATAGCGTCAG | 10 | 541 |
| C1127214 | 14 | 01001011010011101100 | ATCGATACTA | 10 | 542 |
| C1127214 | 14 | 00110101001001110011 | CGAGCACGCG | 10 | 543 |
| C1127214 | 14 | 00111001010101010110 | CGTGAGAGAC | 10 | 544 |
| C1127214 | 14 | 00111001101010101100 | CGTGTCTCTA | 10 | 545 |
| C1127214 | 14 | 00101111010110011000 | CTACGAGTGT | 10 | 546 |
| C1127214 | 14 | 00101010111100100110 | CTCTACGCAC | 10 | 547 |
| C1127214 | 14 | 11101001011001001010 | TACTGACATC | 10 | 548 |
| C1127214 | 14 | 10100110010011010101 | TCACATAGAG | 10 | 549 |
| C1127214 | 14 | 10101001100110010011 | TCTGTGTGCG | 10 | 550 |
| C1127214 | 14 | 10010100110110110010 | TGATAGTCGC | 10 | 551 |
| C1127214 | 14 | 10011011001101001001 | TGTCGCGATG | 10 | 552 |
| C1127215 | 14 | 01100100111010111000 | ACATACTCGT | 10 | 553 |
| C1127215 | 14 | 01010010101111010100 | AGCTCGTAGA | 10 | 554 |
| C1127215 | 14 | 01011100100110010011 | AGTATGTGCG | 10 | 555 |
| C1127215 | 14 | 01001010100101111010 | ATCTGACGTC | 10 | 556 |
| C1127215 | 14 | 00110101001001110011 | CGAGCACGCG | 10 | 557 |
| C1127215 | 14 | 00111001010101010110 | CGTGAGAGAC | 10 | 558 |
| C1127215 | 14 | 00111001101010101100 | CGTGTCTCTA | 10 | 559 |
| C1127215 | 14 | 00101111010110011000 | CTACGAGTGT | 10 | 560 |
| C1127215 | 14 | 00101010111001100101 | CTCTACACAG | 10 | 561 |
| C1127215 | 14 | 11100100110101001100 | TACATAGATA | 10 | 562 |
| C1127215 | 14 | 11010011100100100110 | TAGCGTGCAC | 10 | 563 |
| C1127215 | 14 | 10100110100110101001 | TCACTGTCTG | 10 | 564 |
| C1127215 | 14 | 10111010010011101000 | TCGTCATACT | 10 | 565 |
| C1127215 | 14 | 10011011001101001001 | TGTCGCGATG | 10 | 566 |
| C1127216 | 14 | 01100100111010110010 | ACATACTCGC | 10 | 567 |
| C1127216 | 14 | 01010010101111010100 | AGCTCGTAGA | 10 | 568 |
| C1127216 | 14 | 01011100100110010011 | AGTATGTGCG | 10 | 569 |
| C1127216 | 14 | 01001010100101111010 | ATCTGACGTC | 10 | 570 |
| C1127216 | 14 | 00110101001001110011 | CGAGCACGCG | 10 | 571 |
| C1127216 | 14 | 00111001010101010110 | CGTGAGAGAC | 10 | 572 |
| C1127216 | 14 | 00111001101010101100 | CGTGTCTCTA | 10 | 573 |
| C1127216 | 14 | 00101111010110011000 | CTACGAGTGT | 10 | 574 |
| C1127216 | 14 | 00101010111001100101 | CTCTACACAG | 10 | 575 |
| C1127216 | 14 | 11100100110101001100 | TACATAGATA | 10 | 576 |
| C1127216 | 14 | 11010011100100100110 | TAGCGTGCAC | 10 | 577 |
| C1127216 | 14 | 10100110100110101001 | TCACTGTCTG | 10 | 578 |
| C1127216 | 14 | 10111010010011101000 | TCGTCATACT | 10 | 579 |
| C1127216 | 14 | 10011011001101001001 | TGTCGCGATG | 10 | 580 |
| C1127217 | 14 | 01100110011001101010 | ACACACACTC | 10 | 581 |
| C1127217 | 14 | 01100100101010011101 | ACATCTGTAG | 10 | 582 |
| C1127217 | 14 | 01110011001110011000 | ACGCGCGTGT | 10 | 583 |
| C1127217 | 14 | 01010011010011001110 | AGCGATATAC | 10 | 584 |
| C1127217 | 14 | 01011101100101010100 | AGTAGTGAGA | 10 | 585 |
| C1127217 | 14 | 00110101010100110011 | CGAGAGCGCG | 10 | 586 |
| C1127217 | 14 | 00111010010011111000 | CGTCATACGT | 10 | 587 |
| C1127217 | 14 | 00111001101010101100 | CGTGTCTCTA | 10 | 588 |
| C1127217 | 14 | 00101110110110101000 | CTACTAGTCT | 10 | 589 |
| C1127217 | 14 | 00101011001101100101 | CTCGCGACAG | 10 | 590 |
| C1127217 | 14 | 11010110101101001000 | TAGACTCGAT | 10 | 591 |
| C1127217 | 14 | 11001001010101011010 | TATGAGAGTC | 10 | 592 |
| C1127217 | 14 | 10010010100111010011 | TGCTGTAGCG | 10 | 593 |
| C1127217 | 14 | 10011111001010010010 | TGTACGCTGC | 10 | 594 |
| C1127218 | 14 | 01100100101010011101 | ACATCTGTAG | 10 | 595 |
| C1127218 | 14 | 01110011001110011000 | ACGCGCGTGT | 10 | 596 |
| C1127218 | 14 | 01010011010011001110 | AGCGATATAC | 10 | 597 |
| C1127218 | 14 | 01011101100101010100 | AGTAGTGAGA | 10 | 598 |
| C1127218 | 14 | 00110101010100110011 | CGAGAGCGCG | 10 | 599 |
| C1127218 | 14 | 00111010010011111000 | CGTCATACGT | 10 | 600 |
| C1127218 | 14 | 00111001101010101100 | CGTGTCTCTA | 10 | 601 |
| C1127218 | 14 | 00101110110110101000 | CTACTAGTCT | 10 | 602 |
| C1127218 | 14 | 00101011001101100101 | CTCGCGACAG | 10 | 603 |
| C1127218 | 14 | 11010110101101001000 | TAGACTCGAT | 10 | 604 |
| C1127218 | 14 | 11001100111001100100 | TATATACACA | 10 | 605 |
| C1127218 | 14 | 10100110010101011100 | TCACAGAGTA | 10 | 606 |
| C1127218 | 14 | 10010010100111010011 | TGCTGTAGCG | 10 | 607 |
| C1127218 | 14 | 10011111001010010010 | TGTACGCTGC | 10 | 608 |
| C1127219 | 14 | 01110011001110011000 | ACGCGCGTGT | 10 | 609 |
| C1127219 | 14 | 01010101011001001110 | AGAGACATAC | 10 | 610 |
| C1127219 | 14 | 01001101110110101000 | ATAGTAGTCT | 10 | 611 |
| C1127219 | 14 | 01001010100101110101 | ATCTGACGAG | 10 | 612 |
| C1127219 | 14 | 00100111001011001011 | CACGCTATCG | 10 | 613 |
| C1127219 | 14 | 00110101010100110011 | CGAGAGCGCG | 10 | 614 |
| C1127219 | 14 | 00111010010011111000 | CGTCATACGT | 10 | 615 |
| C1127219 | 14 | 00111001101010101100 | CGTGTCTCTA | 10 | 616 |
| C1127219 | 14 | 00101010011110100110 | CTCACGTCAC | 10 | 617 |
| C1127219 | 14 | 11010010101010010110 | TAGCTCTGAC | 10 | 618 |
| C1127219 | 14 | 11001110010010011001 | TATACATGTG | 10 | 619 |
| C1127219 | 14 | 10100110100101011100 | TCACTGAGTA | 10 | 620 |
| C1127219 | 14 | 10101100111001100100 | TCTATACACA | 10 | 621 |
| C1127219 | 14 | 10010011110101001001 | TGCGTAGATG | 10 | 622 |
| C1127220 | 14 | 01100110010011001101 | ACACATATAG | 10 | 623 |
| C1127220 | 14 | 01110011001110011000 | ACGCGCGTGT | 10 | 624 |
| C1127220 | 14 | 01010111001001101010 | AGACGCACTC | 10 | 625 |
| C1127220 | 14 | 01001100111110100100 | ATATACGTCA | 10 | 626 |
| C1127220 | 14 | 01001001101011010011 | ATGTCTAGCG | 10 | 627 |
| C1127220 | 14 | 00110101010100110011 | CGAGAGCGCG | 10 | 628 |
| C1127220 | 14 | 00110010101101100101 | CGCTCGACAG | 10 | 629 |
| C1127220 | 14 | 00111001101010101100 | CGTGTCTCTA | 10 | 630 |
| C1127220 | 14 | 00101011110101001010 | CTCGTAGATC | 10 | 631 |
| C1127220 | 14 | 11101001010100101100 | TACTGAGCTA | 10 | 632 |
| C1127220 | 14 | 11010011010010010110 | TAGCGATGAC | 10 | 633 |
| C1127220 | 14 | 10101100100110011001 | TCTATGTGTG | 10 | 634 |
| C1127220 | 14 | 10101010011010110010 | TCTCACTCGC | 10 | 635 |
| C1127220 | 14 | 10010101111001011000 | TGAGTACAGT | 10 | 636 |
| C1127221 | 14 | 01100110010011001101 | ACACATATAG | 10 | 637 |
| C1127221 | 14 | 01110011001110011000 | ACGCGCGTGT | 10 | 638 |
| C1127221 | 14 | 01010111001001101010 | AGACGCACTC | 10 | 639 |
| C1127221 | 14 | 01001100111110100100 | ATATACGTCA | 10 | 640 |
| C1127221 | 14 | 01001001101011010011 | ATGTCTAGCG | 10 | 641 |
| C1127221 | 14 | 00110101010100110011 | CGAGAGCGCG | 10 | 642 |
| C1127221 | 14 | 00110010101101100101 | CGCTCGACAG | 10 | 643 |
| C1127221 | 14 | 00111001101010101100 | CGTGTCTCTA | 10 | 644 |
| C1127221 | 14 | 00101011110101001010 | CTCGTAGATC | 10 | 645 |
| C1127221 | 14 | 11101001010100101100 | TACTGAGCTA | 10 | 646 |
| C1127221 | 14 | 11010011010010010110 | TAGCGATGAC | 10 | 647 |
| C1127221 | 14 | 10101100100110011001 | TCTATGTGTG | 10 | 648 |
| C1127221 | 14 | 10101010011010110010 | TCTCACTCGC | 10 | 649 |
| C1127221 | 14 | 10010101111001010100 | TGAGTACAGA | 10 | 650 |
| C1127222 | 14 | 01100100110011100101 | ACATATACAG | 10 | 651 |
| C1127222 | 14 | 01110011001110011000 | ACGCGCGTGT | 10 | 652 |
| C1127222 | 14 | 01010111001001101010 | AGACGCACTC | 10 | 653 |
| C1127222 | 14 | 01001001011011010011 | ATGACTAGCG | 10 | 654 |
| C1127222 | 14 | 00100110010111011100 | CACAGTAGTA | 10 | 655 |
| C1127222 | 14 | 00110101010100110011 | CGAGAGCGCG | 10 | 656 |
| C1127222 | 14 | 00110010111010010110 | CGCTACTGAC | 10 | 657 |
| C1127222 | 14 | 00111001101010101100 | CGTGTCTCTA | 10 | 658 |
| C1127222 | 14 | 00101011001101100101 | CTCGCGACAG | 10 | 659 |
| C1127222 | 14 | 11100100101101010010 | TACATCGAGC | 10 | 660 |
| C1127222 | 14 | 11011110101110000000 | TAGTACTCGT | 10 | 661 |
| C1127222 | 14 | 10101110011010100100 | TCTACACTCA | 10 | 662 |
| C1127222 | 14 | 10101100100110011001 | TCTATGTGTG | 10 | 663 |
| C1127222 | 14 | 10011010010101001110 | TGTCAGATAC | 10 | 664 |
| C1127223 | 14 | 01100110011001101010 | ACACACACTC | 10 | 665 |
| C1127223 | 14 | 01100100101010011101 | ACATCTGTAG | 10 | 666 |
| C1127223 | 14 | 01110011001110011000 | ACGCGCGTGT | 10 | 667 |
| C1127223 | 14 | 01010011010011001110 | AGCGATATAC | 10 | 668 |
| C1127223 | 14 | 01011101100101010100 | AGTAGTGAGA | 10 | 669 |
| C1127223 | 14 | 00110101010100110011 | CGAGAGCGCG | 10 | 670 |
| C1127223 | 14 | 00111001101010101100 | CGTGTCTCTA | 10 | 671 |
| C1127223 | 14 | 00101110110110101000 | CTACTAGTCT | 10 | 672 |
| C1127223 | 14 | 00101011001101100101 | CTCGCGACAG | 10 | 673 |
| C1127223 | 14 | 11010110101101001000 | TAGACTCGAT | 10 | 674 |
| C1127223 | 14 | 11001010010010100111 | TATCATCACG | 10 | 675 |
| C1127223 | 14 | 11001001010101011010 | TATGAGAGTC | 10 | 676 |
| C1127223 | 14 | 10010010100111010011 | TGCTGTAGCG | 10 | 677 |
| C1127223 | 14 | 10011111001010010010 | TGTACGCTGC | 10 | 678 |
| C1127224 | 14 | 01100110101010100101 | ACACTCTCAG | 10 | 679 |
| C1127224 | 14 | 01110011001110011000 | ACGCGCGTGT | 10 | 680 |
| C1127224 | 14 | 01111101011001001000 | ACGTAGACAT | 10 | 681 |
| C1127224 | 14 | 01010110010111001100 | AGACAGTATA | 10 | 682 |
| C1127224 | 14 | 01011100100110010011 | AGTATGTGCG | 10 | 683 |
| C1127224 | 14 | 00110101001100100111 | CGAGCGCACG | 10 | 684 |
| C1127224 | 14 | 00111001101010101100 | CGTGTCTCTA | 10 | 685 |
| C1127224 | 14 | 00101111010010101010 | CTACGATCTC | 10 | 686 |
| C1127224 | 14 | 00101010111101010100 | CTCTACGAGA | 10 | 687 |
| C1127224 | 14 | 11011010011100100100 | TAGTCACGCA | 10 | 688 |
| C1127224 | 14 | 11001101001001011001 | TATAGCAGTG | 10 | 689 |
| C1127224 | 14 | 10111010100101001010 | TCGTCTGATC | 10 | 690 |
| C1127224 | 14 | 10101001010011001101 | TCTGATATAG | 10 | 691 |
| C1127224 | 14 | 10010010011011111000 | TGCACTACGT | 10 | 692 |
| C1127225 | 14 | 01100101101100100110 | ACAGTCGCAC | 10 | 693 |
| C1127225 | 14 | 01110011001110011000 | ACGCGCGTGT | 10 | 694 |
| C1127225 | 14 | 01010110100110010101 | AGACTGTGAG | 10 | 695 |
| C1127225 | 14 | 01011100111001001001 | AGTATACATG | 10 | 696 |
| C1127225 | 14 | 00100110111011010010 | CACTACTAGC | 10 | 697 |
| C1127225 | 14 | 00110101001001110011 | CGAGCACGCG | 10 | 698 |
| C1127225 | 14 | 00111001101010101100 | CGTGTCTCTA | 10 | 699 |
| C1127225 | 14 | 00101111001101001100 | CTACGCGATA | 10 | 700 |
| C1127225 | 14 | 00101010010110011011 | CTCAGTGTCG | 10 | 701 |
| C1127225 | 14 | 11001011001010100101 | TATCGCTCAG | 10 | 702 |
| C1127225 | 14 | 11001001110110010100 | TATGTAGTGA | 10 | 703 |
| C1127225 | 14 | 10110010010011110100 | TCGCATACGA | 10 | 704 |
| C1127225 | 14 | 10010101010011001110 | TGAGATATAC | 10 | 705 |
| C1127225 | 14 | 10011011010101101000 | TGTCGAGACT | 10 | 706 |
| C1127226 | 14 | 01100100111011001001 | ACATACTATG | 10 | 707 |
| C1127226 | 14 | 01110011001110011000 | ACGCGCGTGT | 10 | 708 |
| C1127226 | 14 | 01011101010011011000 | AGTAGATAGT | 10 | 709 |
| C1127226 | 14 | 01001001100101110110 | ATGTGACGAC | 10 | 710 |
| C1127226 | 14 | 00100101010010111110 | CAGATCGTAC | 10 | 711 |
| C1127226 | 14 | 00110101001001110011 | CGAGCACGCG | 10 | 712 |
| C1127226 | 14 | 00111001101010101100 | CGTGTCTCTA | 10 | 713 |
| C1127226 | 14 | 00101111001101001100 | CTACGCGATA | 10 | 714 |
| C1127226 | 14 | 00101010010110011011 | CTCAGTGTCG | 10 | 715 |
| C1127226 | 14 | 11010110011010010100 | TAGACACTGA | 10 | 716 |
| C1127226 | 14 | 11001010110100111000 | TATCTAGCGT | 10 | 717 |
| C1127226 | 14 | 10111100100101010100 | TCGTATGAGA | 10 | 718 |
| C1127226 | 14 | 10101011001010100110 | TCTCGCTCAC | 10 | 719 |
| C1127226 | 14 | 10010010110011101010 | TGCTATACTC | 10 | 720 |
| C1127227 | 14 | 01100101101100100110 | ACAGTCGCAC | 10 | 721 |
| C1127227 | 14 | 01110011001110011000 | ACGCGCGTGT | 10 | 722 |
| C1127227 | 14 | 01011100110101100100 | AGTATAGACA | 10 | 723 |
| C1127227 | 14 | 01001001011011100101 | ATGACTACAG | 10 | 724 |
| C1127227 | 14 | 00100111110101001001 | CACGTAGATG | 10 | 725 |
| C1127227 | 14 | 00110101010100110101 | CGAGAGCGAG | 10 | 726 |
| C1127227 | 14 | 00111010010011111000 | CGTCATACGT | 10 | 727 |
| C1127227 | 14 | 00111001101010101100 | CGTGTCTCTA | 10 | 728 |
| C1127227 | 14 | 00101010011100101011 | CTCACGCTCG | 10 | 729 |
| C1127227 | 14 | 11110100101001101000 | TACGATCACT | 10 | 730 |
| C1127227 | 14 | 11010010010010011101 | TAGCATGTAG | 10 | 731 |
| C1127227 | 14 | 11001110010100110010 | TATACAGCGC | 10 | 732 |
| C1127227 | 14 | 10110101010011001010 | TCGAGATATC | 10 | 733 |
| C1127227 | 14 | 10101001100110010011 | TCTGTGTGCG | 10 | 734 |
| C1127228 | 14 | 01100101101100100110 | ACAGTCGCAC | 10 | 735 |
| C1127228 | 14 | 01110011001110011000 | ACGCGCGTGT | 10 | 736 |
| C1127228 | 14 | 01010110100101011010 | AGACTGAGTC | 10 | 737 |
| C1127228 | 14 | 01001010111001010101 | ATCTACAGAG | 10 | 738 |
| C1127228 | 14 | 00100111110101001001 | CACGTAGATG | 10 | 739 |
| C1127228 | 14 | 00110101010100110101 | CGAGAGCGAG | 10 | 740 |
| C1127228 | 14 | 00111010010011111000 | CGTCATACGT | 10 | 741 |
| C1127228 | 14 | 00111001101010101100 | CGTGTCTCTA | 10 | 742 |
| C1127228 | 14 | 00101010011100101011 | CTCACGCTCG | 10 | 743 |
| C1127228 | 14 | 11110100101001101000 | TACGATCACT | 10 | 744 |
| C1127228 | 14 | 11010010010010011101 | TAGCATGTAG | 10 | 745 |
| C1127228 | 14 | 11001111001001001100 | TATACGCATA | 10 | 746 |
| C1127228 | 14 | 10110101010011001010 | TCGAGATATC | 10 | 747 |
| C1127228 | 14 | 10101001100110010011 | TCTGTGTGCG | 10 | 748 |
| C1127229 | 14 | 01100110110010101001 | ACACTATCTG | 10 | 749 |
| C1127229 | 14 | 01110011001110011000 | ACGCGCGTGT | 10 | 750 |
| C1127229 | 14 | 01010011010011001110 | AGCGATATAC | 10 | 751 |
| C1127229 | 14 | 01011010101101110000 | AGTCTCGACG | 10 | 752 |
| C1127229 | 14 | 01001110010101011010 | ATACAGAGTC | 10 | 753 |
| C1127229 | 14 | 00100111100101110010 | CACGTGACGC | 10 | 754 |
| C1127229 | 14 | 00110101010100110101 | CGAGAGCGAG | 10 | 755 |
| C1127229 | 14 | 00111001101010101100 | CGTGTCTCTA | 10 | 756 |
| C1127229 | 14 | 00101010110111010100 | CTCTAGTAGA | 10 | 757 |
| C1127229 | 14 | 11011010010111001000 | TAGTCAGTAT | 10 | 758 |
| C1127229 | 14 | 11001100111001100100 | TATATACACA | 10 | 759 |
| C1127229 | 14 | 10101011001010100110 | TCTCGCTCAC | 10 | 760 |
| C1127229 | 14 | 10101001100110010011 | TCTGTGTGCG | 10 | 761 |
| C1127229 | 14 | 10010101001011111000 | TGAGCTACGT | 10 | 762 |
| C1127230 | 14 | 01110011001110010100 | ACGCGCGTGA | 10 | 763 |
| C1127230 | 14 | 01101001110101010010 | ACTGTAGAGC | 10 | 764 |
| C1127230 | 14 | 01010101011001001110 | AGAGACATAC | 10 | 765 |
| C1127230 | 14 | 01001111100100101010 | ATACGTGCTC | 10 | 766 |
| C1127230 | 14 | 00100110111001101001 | CACTACACTG | 10 | 767 |
| C1127230 | 14 | 00110101010100110011 | CGAGAGCGCG | 10 | 768 |
| C1127230 | 14 | 00111010010011111000 | CGTCATACGT | 10 | 769 |
| C1127230 | 14 | 00111001101010101100 | CGTGTCTCTA | 10 | 770 |
| C1127230 | 14 | 00101010010110011101 | CTCAGTGTAG | 10 | 771 |
| C1127230 | 14 | 11010100110110011000 | TAGATAGTGT | 10 | 772 |
| C1127230 | 14 | 11001011011100100100 | TATCGACGCA | 10 | 773 |
| C1127230 | 14 | 10101111010011001000 | TCTACGATAT | 10 | 774 |
| C1127230 | 14 | 10101100101010010011 | TCTATCTGCG | 10 | 775 |
| C1127230 | 14 | 10010010011010110110 | TGCACTCGAC | 10 | 776 |
| C1127231 | 14 | 01110011001110010100 | ACGCGCGTGA | 10 | 777 |
| C1127231 | 14 | 01010111001001101010 | AGACGCACTC | 10 | 778 |
| C1127231 | 14 | 01011101010111001000 | AGTAGAGTAT | 10 | 779 |
| C1127231 | 14 | 01001011100101100101 | ATCGTGACAG | 10 | 780 |
| C1127231 | 14 | 01001001011011010011 | ATGACTAGCG | 10 | 781 |
| C1127231 | 14 | 00100110011010011101 | CACACTGTAG | 10 | 782 |
| C1127231 | 14 | 00110101010100110011 | CGAGAGCGCG | 10 | 783 |
| C1127231 | 14 | 00111010010011111000 | CGTCATACGT | 10 | 784 |
| C1127231 | 14 | 00111001101010101100 | CGTGTCTCTA | 10 | 785 |
| C1127231 | 14 | 11011010011100100100 | TAGTCACGCA | 10 | 786 |
| C1127231 | 14 | 11001010110101001010 | TATCTAGATC | 10 | 787 |
| C1127231 | 14 | 10100111010011100100 | TCACGATACA | 10 | 788 |
| C1127231 | 14 | 10101100100110011001 | TCTATGTGTG | 10 | 789 |
| C1127231 | 14 | 10010010010110110110 | TGCAGTCGAC | 10 | 790 |
| C1127232 | 14 | 01110011001110010100 | ACGCGCGTGA | 10 | 791 |
| C1127232 | 14 | 01101001010111101000 | ACTGAGTACT | 10 | 792 |
| C1127232 | 14 | 01010100101111001010 | AGATCGTATC | 10 | 793 |
| C1127232 | 14 | 01001110101010100101 | ATACTCTCAG | 10 | 794 |
| C1127232 | 14 | 01001001111001010011 | ATGTACAGCG | 10 | 795 |
| C1127232 | 14 | 00110101010100110011 | CGAGAGCGCG | 10 | 796 |
| C1127232 | 14 | 00110010010111001101 | CGCAGTATAG | 10 | 797 |
| C1127232 | 14 | 00111001101010101100 | CGTGTCTCTA | 10 | 798 |
| C1127232 | 14 | 00101111011001001100 | CTACGACATA | 10 | 799 |
| C1127232 | 14 | 11010101011010011000 | TAGAGACTGT | 10 | 800 |
| C1127232 | 14 | 11010011100100100110 | TAGCGTGCAC | 10 | 801 |
| C1127232 | 14 | 10111010110101001000 | TCGTCTAGAT | 10 | 802 |
| C1127232 | 14 | 10101100100110011001 | TCTATGTGTG | 10 | 803 |
| C1127232 | 14 | 10101010011010110010 | TCTCACTCGC | 10 | 804 |
| C1127233 | 14 | 01100101101100100110 | ACAGTCGCAC | 10 | 805 |
| C1127233 | 14 | 01101001011001011001 | ACTGACAGTG | 10 | 806 |
| C1127233 | 14 | 01010100110101101100 | AGATAGACTA | 10 | 807 |
| C1127233 | 14 | 01010011001011100101 | AGCGCTACAG | 10 | 808 |
| C1127233 | 14 | 01001010110010101011 | ATCTATCTCG | 10 | 809 |
| C1127233 | 14 | 00110101010100110011 | CGAGAGCGCG | 10 | 810 |
| C1127233 | 14 | 00111010010011111000 | CGTCATACGT | 10 | 811 |
| C1127233 | 14 | 00111001101010101100 | CGTGTCTCTA | 10 | 812 |
| C1127233 | 14 | 00101111100101100100 | CTACGTGACA | 10 | 813 |
| C1127233 | 14 | 11011110100100101000 | TAGTACTGCT | 10 | 814 |
| C1127233 | 14 | 11001010101001010110 | TATCTCAGAC | 10 | 815 |
| C1127233 | 14 | 10100101010111010100 | TCAGAGTAGA | 10 | 816 |
| C1127233 | 14 | 10110010100110011001 | TCGCTGTGTG | 10 | 817 |
| C1127233 | 14 | 10010011011101001010 | TGCGACGATC | 10 | 818 |
| C1127234 | 14 | 01100110011010011001 | ACACACTGTG | 10 | 819 |
| C1127234 | 14 | 01110010110101001100 | ACGCTAGATA | 10 | 820 |
| C1127234 | 14 | 01101001100100111010 | ACTGTGCGTC | 10 | 821 |
| C1127234 | 14 | 01001111001001110100 | ATACGCACGA | 10 | 822 |
| C1127234 | 14 | 00110101010100110011 | CGAGAGCGCG | 10 | 823 |
| C1127234 | 14 | 00110010011110100110 | CGCACGTCAC | 10 | 824 |
| C1127234 | 14 | 00111010010011111000 | CGTCATACGT | 10 | 825 |
| C1127234 | 14 | 00111001101010101100 | CGTGTCTCTA | 10 | 826 |
| C1127234 | 14 | 00101110110110101000 | CTACTAGTCT | 10 | 827 |
| C1127234 | 14 | 11100100101101010010 | TACATCGAGC | 10 | 828 |
| C1127234 | 14 | 11011001110011100000 | TAGTGTATAC | 10 | 829 |
| C1127234 | 14 | 10101100100101100101 | TCTATGACAG | 10 | 830 |
| C1127234 | 14 | 10010100111001101010 | TGATACACTC | 10 | 831 |
| C1127234 | 14 | 10011011010110010100 | TGTCGAGTGA | 10 | 832 |
| C1127235 | 14 | 01110100101010010101 | ACGATCTGAG | 10 | 833 |
| C1127235 | 14 | 01101011001100101001 | ACTCGCGCTG | 10 | 834 |
| C1127235 | 14 | 01011100110101100100 | AGTATAGACA | 10 | 835 |
| C1127235 | 14 | 01001110101001001011 | ATACTCATCG | 10 | 836 |
| C1127235 | 14 | 01001001100101111010 | ATGTGACGTC | 10 | 837 |
| C1127235 | 14 | 00100111110111001000 | CACGTAGTAT | 10 | 838 |
| C1127235 | 14 | 00110101010100110011 | CGAGAGCGCG | 10 | 839 |
| C1127235 | 14 | 00110010011110100110 | CGCACGTCAC | 10 | 840 |
| C1127235 | 14 | 00111010010011111000 | CGTCATACGT | 10 | 841 |
| C1127235 | 14 | 00111001101010101100 | CGTGTCTCTA | 10 | 842 |
| C1127235 | 14 | 11010100110010101010 | TAGATATCTC | 10 | 843 |
| C1127235 | 14 | 11010010011101011000 | TAGCACGAGT | 10 | 844 |
| C1127235 | 14 | 11001101100110010100 | TATAGTGTGA | 10 | 845 |
| C1127235 | 14 | 10101010100101001110 | TCTCTGATAC | 10 | 846 |
| C1127236 | 14 | 01100100101010111001 | ACATCTCGTG | 10 | 847 |
| C1127236 | 14 | 01110010100101100101 | ACGCTGACAG | 10 | 848 |
| C1127236 | 14 | 01010111001001101010 | AGACGCACTC | 10 | 849 |
| C1127236 | 14 | 01011110110010011000 | AGTACTATGT | 10 | 850 |
| C1127236 | 14 | 01001011010101011100 | ATCGAGAGTA | 10 | 851 |
| C1127236 | 14 | 00100101001110011110 | CAGCGTGTAC | 10 | 852 |
| C1127236 | 14 | 00110101010100110011 | CGAGAGCGCG | 10 | 853 |
| C1127236 | 14 | 00111001101010101100 | CGTGTCTCTA | 10 | 854 |
| C1127236 | 14 | 00101010011001110110 | CTCACACGAC | 10 | 855 |
| C1127236 | 14 | 11100101010011010010 | TACAGATAGC | 10 | 856 |
| C1127236 | 14 | 11011010011100100100 | TAGTCACGCA | 10 | 857 |
| C1127236 | 14 | 10101110100100101010 | TCTACTGCTC | 10 | 858 |
| C1127236 | 14 | 10010011001110111000 | TGCGCGTCGT | 10 | 859 |
| C1127236 | 14 | 10011100101001010101 | TGTATCAGAG | 10 | 860 |
| C1127237 | 14 | 01101010110010100110 | ACTCTATCAC | 10 | 861 |
| C1127237 | 14 | 01101001010011011001 | ACTGATAGTG | 10 | 862 |
| C1127237 | 14 | 01010111001001101010 | AGACGCACTC | 10 | 863 |
| C1127237 | 14 | 01011101100101010100 | AGTAGTGAGA | 10 | 864 |
| C1127237 | 14 | 00100101001110011110 | CAGCGTGTAC | 10 | 865 |
| C1127237 | 14 | 00110101010100110011 | CGAGAGCGCG | 10 | 866 |
| C1127237 | 14 | 00111001101010101100 | CGTGTCTCTA | 10 | 867 |
| C1127237 | 14 | 00101010101101010101 | CTCTCGAGAG | 10 | 868 |
| C1127237 | 14 | 11100101011101100000 | TACAGACGAC | 10 | 869 |
| C1127237 | 14 | 11011101010010101000 | TAGTAGATCT | 10 | 870 |
| C1127237 | 14 | 11001010011010010011 | TATCACTGCG | 10 | 871 |
| C1127237 | 14 | 10101110011001001100 | TCTACACATA | 10 | 872 |
| C1127237 | 14 | 10010100100111001101 | TGATGTATAG | 10 | 873 |
| C1127237 | 14 | 10010011001110111000 | TGCGCGTCGT | 10 | 874 |
| C1127238 | 14 | 01101010110010100110 | ACTCTATCAC | 10 | 875 |
| C1127238 | 14 | 01101001010011011001 | ACTGATAGTG | 10 | 876 |
| C1127238 | 14 | 01010111001001101010 | AGACGCACTC | 10 | 877 |
| C1127238 | 14 | 01011101100101010100 | AGTAGTGAGA | 10 | 878 |
| C1127238 | 14 | 00100101001110011110 | CAGCGTGTAC | 10 | 879 |
| C1127238 | 14 | 00110101010100110011 | CGAGAGCGCG | 10 | 880 |
| C1127238 | 14 | 00111001101010101100 | CGTGTCTCTA | 10 | 881 |
| C1127238 | 14 | 00101010101101010101 | CTCTCGAGAG | 10 | 882 |
| C1127238 | 14 | 11100110100100101001 | TACACTGCTG | 10 | 883 |
| C1127238 | 14 | 11100101011101100000 | TACAGACGAC | 10 | 884 |
| C1127238 | 14 | 11011101010010101000 | TAGTAGATCT | 10 | 885 |
| C1127238 | 14 | 10101110011001001100 | TCTACACATA | 10 | 886 |
| C1127238 | 14 | 10010100100111001101 | TGATGTATAG | 10 | 887 |
| C1127238 | 14 | 10010011001110111000 | TGCGCGTCGT | 10 | 888 |
| C1127239 | 14 | 01110010010010101110 | ACGCATCTAC | 10 | 889 |
| C1127239 | 14 | 01101011001100101001 | ACTCGCGCTG | 10 | 890 |
| C1127239 | 14 | 01010110100110011010 | AGACTGTGTC | 10 | 891 |
| C1127239 | 14 | 01001100111100100110 | ATATACGCAC | 10 | 892 |
| C1127239 | 14 | 00100111110110010100 | CACGTAGTGA | 10 | 893 |
| C1127239 | 14 | 00110101010100110011 | CGAGAGCGCG | 10 | 894 |
| C1127239 | 14 | 00111011001001010110 | CGTCGCAGAC | 10 | 895 |
| C1127239 | 14 | 00111001101010101100 | CGTGTCTCTA | 10 | 896 |
| C1127239 | 14 | 00101010111001001011 | CTCTACATCG | 10 | 897 |
| C1127239 | 14 | 11100101010011010010 | TACAGATAGC | 10 | 898 |
| C1127239 | 14 | 11011010100101100100 | TAGTCTGACA | 10 | 899 |
| C1127239 | 14 | 10101100101101011000 | TCTATCGAGT | 10 | 900 |
| C1127239 | 14 | 10010100110011101001 | TGATATACTG | 10 | 901 |
| C1127239 | 14 | 10010011001110111000 | TGCGCGTCGT | 10 | 902 |
| C1127240 | 14 | 01101011100110010100 | ACTCGTGTGA | 10 | 903 |
| C1127240 | 14 | 01101001011001111000 | ACTGACACGT | 10 | 904 |
| C1127240 | 14 | 01010010011011101010 | AGCACTACTC | 10 | 905 |
| C1127240 | 14 | 01001110010101001110 | ATACAGATAC | 10 | 906 |
| C1127240 | 14 | 01001100100110111010 | ATATGTCGTC | 10 | 907 |
| C1127240 | 14 | 00110101010100110011 | CGAGAGCGCG | 10 | 908 |
| C1127240 | 14 | 00111011001001010110 | CGTCGCAGAC | 10 | 909 |
| C1127240 | 14 | 00111001101010101100 | CGTGTCTCTA | 10 | 910 |
| C1127240 | 14 | 00101010111111001000 | CTCTACGTAT | 10 | 911 |
| C1127240 | 14 | 11011010011100100100 | TAGTCACGCA | 10 | 912 |
| C1127240 | 14 | 10100110011011010100 | TCACACTAGA | 10 | 913 |
| C1127240 | 14 | 10101100101010010011 | TCTATCTGCG | 10 | 914 |
| C1127240 | 14 | 10010100100111001101 | TGATGTATAG | 10 | 915 |
| C1127240 | 14 | 10010011001110111000 | TGCGCGTCGT | 10 | 916 |
| C1127241 | 14 | 01101011100110010100 | ACTCGTGTGA | 10 | 917 |
| C1127241 | 14 | 01101001001010100111 | ACTGCTCACG | 10 | 918 |
| C1127241 | 14 | 01011100111001011000 | AGTATACAGT | 10 | 919 |
| C1127241 | 14 | 01001110010111100100 | ATACAGTACA | 10 | 920 |
| C1127241 | 14 | 01001101101101001010 | ATAGTCGATC | 10 | 921 |
| C1127241 | 14 | 00100111010011101010 | CACGATACTC | 10 | 922 |
| C1127241 | 14 | 00110101010100110011 | CGAGAGCGCG | 10 | 923 |
| C1127241 | 14 | 00111011001001010110 | CGTCGCAGAC | 10 | 924 |
| C1127241 | 14 | 00111001101010101100 | CGTGTCTCTA | 10 | 925 |
| C1127241 | 14 | 00101010111111001000 | CTCTACGTAT | 10 | 926 |
| C1127241 | 14 | 11001010110100110010 | TATCTAGCGC | 10 | 927 |
| C1127241 | 14 | 11001001010101001101 | TATGAGATAG | 10 | 928 |
| C1127241 | 14 | 10100110011010010101 | TCACACTGAG | 10 | 929 |
| C1127241 | 14 | 10010011001110111000 | TGCGCGTCGT | 10 | 930 |
| C1127242 | 14 | 01101011100110010100 | ACTCGTGTGA | 10 | 931 |
| C1127242 | 14 | 01101001001010100111 | ACTGCTCACG | 10 | 932 |
| C1127242 | 14 | 01011100111001011000 | AGTATACAGT | 10 | 933 |
| C1127242 | 14 | 01001101101101001010 | ATAGTCGATC | 10 | 934 |
| C1127242 | 14 | 00100111010011101010 | CACGATACTC | 10 | 935 |
| C1127242 | 14 | 00110101010100110011 | CGAGAGCGCG | 10 | 936 |
| C1127242 | 14 | 00111011001001010110 | CGTCGCAGAC | 10 | 937 |
| C1127242 | 14 | 00111001101010101100 | CGTGTCTCTA | 10 | 938 |
| C1127242 | 14 | 00101010111111001000 | CTCTACGTAT | 10 | 939 |
| C1127242 | 14 | 11001010110100110010 | TATCTAGCGC | 10 | 940 |
| C1127242 | 14 | 11001001010101001101 | TATGAGATAG | 10 | 941 |
| C1127242 | 14 | 10100110011011010100 | TCACACTAGA | 10 | 942 |
| C1127242 | 14 | 10100101110010011001 | TCAGTATGTG | 10 | 943 |
| C1127242 | 14 | 10010011001110111000 | TGCGCGTCGT | 10 | 944 |
| C1127243 | 14 | 01100100111010100110 | ACATACTCAC | 10 | 945 |
| C1127243 | 14 | 01110010011101001100 | ACGCACGATA | 10 | 946 |
| C1127243 | 14 | 01101001100110010101 | ACTGTGTGAG | 10 | 947 |
| C1127243 | 14 | 01011100111001011000 | AGTATACAGT | 10 | 948 |
| C1127243 | 14 | 01001111110010010010 | ATACGTATGC | 10 | 949 |
| C1127243 | 14 | 00110101010100110011 | CGAGAGCGCG | 10 | 950 |
| C1127243 | 14 | 00111011001001010110 | CGTCGCAGAC | 10 | 951 |
| C1127243 | 14 | 00111001101010101100 | CGTGTCTCTA | 10 | 952 |
| C1127243 | 14 | 00101010111001001011 | CTCTACATCG | 10 | 953 |
| C1127243 | 14 | 11100110100100101001 | TACACTGCTG | 10 | 954 |
| C1127243 | 14 | 11001001010101101010 | TATGAGACTC | 10 | 955 |
| C1127243 | 14 | 10100101001111100100 | TCAGCGTACA | 10 | 956 |
| C1127243 | 14 | 10101011001100111000 | TCTCGCGCGT | 10 | 957 |
| C1127243 | 14 | 10010110110111001000 | TGACTAGTAT | 10 | 958 |
| C1127244 | 14 | 01110010110101101000 | ACGCTAGACT | 10 | 959 |
| C1127244 | 14 | 01011011100101010100 | AGTCGTGAGA | 10 | 960 |
| C1127244 | 14 | 01001100101001001111 | ATATCATACG | 10 | 961 |
| C1127244 | 14 | 00100100110111011001 | CATAGTAGTG | 10 | 962 |
| C1127244 | 14 | 00110101010101001110 | CGAGAGATAC | 10 | 963 |
| C1127244 | 14 | 00111001101010101100 | CGTGTCTCTA | 10 | 964 |
| C1127244 | 14 | 00101011001110011010 | CTCGCGTGTC | 10 | 965 |
| C1127244 | 14 | 00101010111100100101 | CTCTACGCAG | 10 | 966 |
| C1127244 | 14 | 11100110011001001100 | TACACACATA | 10 | 967 |
| C1127244 | 14 | 11101001100111001000 | TACTGTGTAT | 10 | 968 |
| C1127244 | 14 | 11010010101010110010 | TAGCTCTCGC | 10 | 969 |
| C1127244 | 14 | 10101101010010110100 | TCTAGATCGA | 10 | 970 |
| C1127244 | 14 | 10010110010100110011 | TGACAGCGCG | 10 | 971 |
| C1127244 | 14 | 10010101111001011000 | TGAGTACAGT | 10 | 972 |
| C1127245 | 14 | 01110101100100111000 | ACGAGTGCGT | 10 | 973 |
| C1127245 | 14 | 01110010101101100100 | ACGCTCGACA | 10 | 974 |
| C1127245 | 14 | 01010111001001101010 | AGACGCACTC | 10 | 975 |
| C1127245 | 14 | 01010010011010011101 | AGCACTGTAG | 10 | 976 |
| C1127245 | 14 | 01001100101100110101 | ATATCGCGAG | 10 | 977 |
| C1127245 | 14 | 01001010010101100111 | ATCAGACACG | 10 | 978 |
| C1127245 | 14 | 00100100110111011001 | CATAGTAGTG | 10 | 979 |
| C1127245 | 14 | 00110101010101001110 | CGAGAGATAC | 10 | 980 |
| C1127245 | 14 | 00111001101010101100 | CGTGTCTCTA | 10 | 981 |
| C1127245 | 14 | 00101011001110011010 | CTCGCGTGTC | 10 | 982 |
| C1127245 | 14 | 11101001010100101100 | TACTGAGCTA | 10 | 983 |
| C1127245 | 14 | 11011100101001010010 | TAGTATCAGC | 10 | 984 |
| C1127245 | 14 | 10101010110010010011 | TCTCTATGCG | 10 | 985 |
| C1127245 | 14 | 10010100111110101000 | TGATACGTCT | 10 | 986 |
| C1127246 | 14 | 01100110100111001100 | ACACTGTATA | 10 | 987 |
| C1127246 | 14 | 01100101001101111000 | ACAGCGACGT | 10 | 988 |
| C1127246 | 14 | 01100100111010100110 | ACATACTCAC | 10 | 989 |
| C1127246 | 14 | 01001011001100101101 | ATCGCGCTAG | 10 | 990 |
| C1127246 | 14 | 01001001110011011010 | ATGTATAGTC | 10 | 991 |
| C1127246 | 14 | 00110110010010111001 | CGACATCGTG | 10 | 992 |
| C1127246 | 14 | 00110101010101001110 | CGAGAGATAC | 10 | 993 |
| C1127246 | 14 | 00111001101010101100 | CGTGTCTCTA | 10 | 994 |
| C1127246 | 14 | 00101010111101010100 | CTCTACGAGA | 10 | 995 |
| C1127246 | 14 | 11101101010010101000 | TACTAGATCT | 10 | 996 |
| C1127246 | 14 | 11010100100110010101 | TAGATGTGAG | 10 | 997 |
| C1127246 | 14 | 10101001101001010011 | TCTGTCAGCG | 10 | 998 |
| C1127246 | 14 | 10010011001110011010 | TGCGCGTGTC | 10 | 999 |
| C1127246 | 14 | 10011011111001001000 | TGTCGTACAT | 10 | 1000 |
| C1127247 | 14 | 01100110100111001100 | ACACTGTATA | 10 | 1001 |
| C1127247 | 14 | 01010110011010111000 | AGACACTCGT | 10 | 1002 |
| C1127247 | 14 | 01001011001100101101 | ATCGCGCTAG | 10 | 1003 |
| C1127247 | 14 | 01001001110011011010 | ATGTATAGTC | 10 | 1004 |
| C1127247 | 14 | 00110101010101001110 | CGAGAGATAC | 10 | 1005 |
| C1127247 | 14 | 00111010100101010011 | CGTCTGAGCG | 10 | 1006 |
| C1127247 | 14 | 00111001101010101100 | CGTGTCTCTA | 10 | 1007 |
| C1127247 | 14 | 00101111101101100000 | CTACGTCGAC | 10 | 1008 |
| C1127247 | 14 | 11101101001010100100 | TACTAGCTCA | 10 | 1009 |
| C1127247 | 14 | 11010100100110010101 | TAGATGTGAG | 10 | 1010 |
| C1127247 | 14 | 10100101101001110010 | TCAGTCACGC | 10 | 1011 |
| C1127247 | 14 | 10101110010101011000 | TCTACAGAGT | 10 | 1012 |
| C1127247 | 14 | 10010011001110011010 | TGCGCGTGTC | 10 | 1013 |
| C1127247 | 14 | 10011011111001001000 | TGTCGTACAT | 10 | 1014 |
| C1127248 | 14 | 01101010101101100100 | ACTCTCGACA | 10 | 1015 |
| C1127248 | 14 | 01010100111001101001 | AGATACACIG | 10 | 1016 |
| C1127248 | 14 | 01001111110010101000 | ATACGTATCT | 10 | 1017 |
| C1127248 | 14 | 01001001100101111010 | ATGTGACGTC | 10 | 1018 |
| C1127248 | 14 | 00100111011010010101 | CACGACTGAG | 10 | 1019 |
| C1127248 | 14 | 00110101010101001110 | CGAGAGATAC | 10 | 1020 |
| C1127248 | 14 | 00110010011110111000 | CGCACGTCGT | 10 | 1021 |
| C1127248 | 14 | 00111001101010101100 | CGTGTCTCTA | 10 | 1022 |
| C1127248 | 14 | 00101001110011010011 | CTGTATAGCG | 10 | 1023 |
| C1127248 | 14 | 11010100100110011100 | TAGATGTGTA | 10 | 1024 |
| C1127248 | 14 | 11001010010110100110 | TATCAGTCAC | 10 | 1025 |
| C1127248 | 14 | 10110110111010100000 | TCGACTACTC | 10 | 1026 |
| C1127248 | 14 | 10111101001001010100 | TCGTAGCAGA | 10 | 1027 |
| C1127248 | 14 | 10010101001100110011 | TGAGCGCGCG | 10 | 1028 |
| C1127249 | 14 | 01100101001011100101 | ACAGCTACAG | 10 | 1029 |
| C1127249 | 14 | 01010010101010011011 | AGCTCTGTCG | 10 | 1030 |
| C1127249 | 14 | 01001111100100101010 | ATACGTGCTC | 10 | 1031 |
| C1127249 | 14 | 01001001110011111000 | ATGTATACGT | 10 | 1032 |
| C1127249 | 14 | 00100111010110011001 | CACGAGTGTG | 10 | 1033 |
| C1127249 | 14 | 00110101010101001110 | CGAGAGATAC | 10 | 1034 |
| C1127249 | 14 | 00111001101010101100 | CGTGTCTCTA | 10 | 1035 |
| C1127249 | 14 | 00101010011111010100 | CTCACGTAGA | 10 | 1036 |
| C1127249 | 14 | 11010110011010010100 | TAGACACTGA | 10 | 1037 |
| C1127249 | 14 | 11001010110101001100 | TATCTAGATA | 10 | 1038 |
| C1127249 | 14 | 10100100110010110011 | TCATATCGCG | 10 | 1039 |
| C1127249 | 14 | 10101001100110010110 | TCTGTGTGAC | 10 | 1040 |
| C1127249 | 14 | 10010011101101011000 | TGCGTCGAGT | 10 | 1041 |
| C1127249 | 14 | 10011011001001100110 | TGTCGCACAC | 10 | 1042 |
| C1127250 | 14 | 01100110011001111000 | ACACACACGT | 10 | 1043 |
| C1127250 | 14 | 01101010101101001010 | ACTCTCGATC | 10 | 1044 |
| C1127250 | 14 | 01010011001110011001 | AGCGCGTGTG | 10 | 1045 |
| C1127250 | 14 | 01001001011011010011 | ATGACTAGCG | 10 | 1046 |
| C1127250 | 14 | 00110101010101001110 | CGAGAGATAC | 10 | 1047 |
| C1127250 | 14 | 00111010010010010111 | CGTCATGACG | 10 | 1048 |
| C1127250 | 14 | 00111001101010101100 | CGTGTCTCTA | 10 | 1049 |
| C1127250 | 14 | 00101111101010010010 | CTACGTCTGC | 10 | 1050 |
| C1127250 | 14 | 11100101001100101100 | TACAGCGCTA | 10 | 1051 |
| C1127250 | 14 | 11011010010111001000 | TAGTCAGTAT | 10 | 1052 |
| C1127250 | 14 | 10100110100111010100 | TCACTGTAGA | 10 | 1053 |
| C1127250 | 14 | 10101001100100110011 | TCTGTGCGCG | 10 | 1054 |
| C1127250 | 14 | 10010100111010011010 | TGATACTGTC | 10 | 1055 |
| C1127250 | 14 | 10011011001001100110 | TGTCGCACAC | 10 | 1056 |
| C1127251 | 14 | 01100110011001111000 | ACACACACGT | 10 | 1057 |
| C1127251 | 14 | 01101010101101001010 | ACTCTCGATC | 10 | 1058 |
| C1127251 | 14 | 01010011001110011001 | AGCGCGTGTG | 10 | 1059 |
| C1127251 | 14 | 01001001011011010011 | ATGACTAGCG | 10 | 1060 |
| C1127251 | 14 | 00110101010101001110 | CGAGAGATAC | 10 | 1061 |
| C1127251 | 14 | 00111010010010010111 | CGTCATGACG | 10 | 1062 |
| C1127251 | 14 | 00111001101010101100 | CGTGTCTCTA | 10 | 1063 |
| C1127251 | 14 | 00101111101010010010 | CTACGTCTGC | 10 | 1064 |
| C1127251 | 14 | 11100101001100101100 | TACAGCGCTA | 10 | 1065 |
| C1127251 | 14 | 11011010010111001000 | TAGTCAGTAT | 10 | 1066 |
| C1127251 | 14 | 10110100100111010100 | TCGATGTAGA | 10 | 1067 |
| C1127251 | 14 | 10101001100100110011 | TCTGTGCGCG | 10 | 1068 |
| C1127251 | 14 | 10010100111010011010 | TGATACTGTC | 10 | 1069 |
| C1127251 | 14 | 10011011001001100110 | TGTCGCACAC | 10 | 1070 |
| C1127252 | 14 | 01100110011001111000 | ACACACACGT | 10 | 1071 |
| C1127252 | 14 | 01101010101101001010 | ACTCTCGATC | 10 | 1072 |
| C1127252 | 14 | 01010011001110011001 | AGCGCGTGTG | 10 | 1073 |
| C1127252 | 14 | 01001001011011010011 | ATGACTAGCG | 10 | 1074 |
| C1127252 | 14 | 00110101010101001110 | CGAGAGATAC | 10 | 1075 |
| C1127252 | 14 | 00111010010010010111 | CGTCATGACG | 10 | 1076 |
| C1127252 | 14 | 00111001101010101100 | CGTGTCTCTA | 10 | 1077 |
| C1127252 | 14 | 00101111101010010010 | CTACGTCTGC | 10 | 1078 |
| C1127252 | 14 | 11100101001100101100 | TACAGCGCTA | 10 | 1079 |
| C1127252 | 14 | 11011010010111001000 | TAGTCAGTAT | 10 | 1080 |
| C1127252 | 14 | 10101100110011010100 | TCTATATAGA | 10 | 1081 |
| C1127252 | 14 | 10101001100100110011 | TCTGTGCGCG | 10 | 1082 |
| C1127252 | 14 | 10010100111010011010 | TGATACTGTC | 10 | 1083 |
| C1127252 | 14 | 10011011001001100110 | TGTCGCACAC | 10 | 1084 |
| C1127253 | 14 | 01100110011001111000 | ACACACACGT | 10 | 1085 |
| C1127253 | 14 | 01101010101101001010 | ACTCTCGATC | 10 | 1086 |
| C1127253 | 14 | 01010011001110011001 | AGCGCGTGTG | 10 | 1087 |
| C1127253 | 14 | 01001001011011010011 | ATGACTAGCG | 10 | 1088 |
| C1127253 | 14 | 00110101010101001110 | CGAGAGATAC | 10 | 1089 |
| C1127253 | 14 | 00111010010010010111 | CGTCATGACG | 10 | 1090 |
| C1127253 | 14 | 00111001101010101100 | CGTGTCTCTA | 10 | 1091 |
| C1127253 | 14 | 00101111101010010010 | CTACGTCTGC | 10 | 1092 |
| C1127253 | 14 | 11100101001100101100 | TACAGCGCTA | 10 | 1093 |
| C1127253 | 14 | 11011010010111001000 | TAGTCAGTAT | 10 | 1094 |
| C1127253 | 14 | 11001100110011010100 | TATATATAGA | 10 | 1095 |
| C1127253 | 14 | 10101001100100110011 | TCTGTGCGCG | 10 | 1096 |
| C1127253 | 14 | 10010100111010011010 | TGATACTGTC | 10 | 1097 |
| C1127253 | 14 | 10011011001001100110 | TGTCGCACAC | 10 | 1098 |

### Example 4

### Exemplary Computer Code for Representing and Manipulating Nucleotide Sequences for UID Identification

package com.fourfivefour.amplicons;

import java.util.HashSet;
import java.util.Set;
/**
* Code to implement common operations on Nucleotide Sequences
*
*
*
*/
public class Sequence implements Comparable<Sequence> {

private String sequence;
static final char possibleBases[ ] = { ‘A’, ‘C’, ‘T’, ‘G’ };
public Sequence(String sequence) {

this.sequence = sequence.toUpperCase( );

}
public String getSequence( ) {

return sequence;

}
public int hashCode( ) {

return sequence.hashCode( );

}
public boolean equals(Object obj) {

return ((this == obj) ∥

((obj instanceof Sequence) &&
sequence.equals(((Sequence) obj).sequence)));

}
public int compareTo(Sequence obj) {

return sequence.compareTo(obj.sequence);

}
public String toString( ) {

return sequence;

| } | |
|---|---|
| /** | |
| * Generate the set of all single base insertions for the | |
| * Sequence. | |
| * | |
| * @return | A set of Sequences representing all single base |

| | |
|---|---|
| * | insertions of the Sequence. |
| */ | |

public Set<Sequence> generateSingleInsertions( ) {

Set<Sequence> insertions = new HashSet<Sequence>( );
int seqLen = sequence.length( );
for (int insertIdx = 0; insertIdx <= seqLen; insertIdx++) {

String prefixString = sequence.substring(0, insertIdx);
String suffixString = sequence.substring(insertIdx,seqLen);
for (char insertBase : possibleBases) {

insertions.add(new Sequence(prefixString + insertBase +

suffixString));

}

}
return insertions;

| } | |
|---|---|
| /** | |
| * Generate the set of all single base substitutions for the | |
| * Sequence. | |
| * | |
| * @return | A set of Sequences representing all single base |

| | |
|---|---|
| * | substitutions of the Sequence. |
| */ | |

public Set<Sequence> generateSingleSubstitutions( ) {

Set<Sequence> substitutions = new HashSet<Sequence>( );
int seqLen = sequence.length( );
for (int substBaseIdx = 0; substBaseIdx < seqLen; substBaseIdx++) {

String prefixString =

sequence.substring(0, substBaseIdx);

String suffixString =

sequence.substring(substBaseIdx + 1, seqLen);

char originalBase =

sequence.charAt(substBaseIdx);

for (char substBase : possibleBases) {

if (substBase != originalBase) {

substitutions.add(
new Sequence(prefixString + substBase + suffixString)
);

}

}

}
return substitutions;

| } | |
|---|---|
| /** | |
| * Generate the set of all single base deletions for the | |
| * Sequence. | |
| * | |
| * @return | A set of sequences representing all single base |

| | |
|---|---|
| * | deletions of the Sequence. |
| */ | |

public Set<Sequence> generateSingleDeletions( ) {

Set<Sequence> deletions = new HashSet<Sequence>( );
int seqLen = sequence.length( );
for (int deleteBaseIdx = 0; deleteBaseIdx < seqLen; deleteBaseIdx++) {

String prefixString =

sequence.substring(0, deleteBaseIdx);

String suffixString =

sequence.substring(deleteBaseIdx + 1 , seqLen);

deletions.add(new Sequence(prefixString + suffixString));

}
return deletions;

| } | |
|---|---|
| /** | |
| * Generate all 1-base mutations starting from each of the sequences in | |
| * the input set of sequences. | |
| * | |
| * @param inputSeqs The input set of sequences. | |
| * @return A set of sequences that are exactly one mutation | |
| * | away from each of the sequences in the input set |
| * | of sequences. |
| */ | |

public static Set<Sequence> generateSingleMutations(Set<Sequence> inputSeqs) {

Set<Sequence> mutatedSequences = new HashSet<Sequence>( );
for (Sequence inputSeq : inputSeqs) {

mutatedSequences.addAll(inputSeq.generateSingleDeletions( ));
mutatedSequences.addAll(inputSeq.generateSingleInsertions( ));
mutatedSequences.addAll(inputSeq.generateSingleSubstitutions( ));

}
return mutatedSequences;

}

}

As stated previously, it will be appreciated that the foregoing computer code is provided for the purposes of example, and that numerous alternative methods and code structures may be employed. It will also be appreciated that the exemplary code provided herein is not intended to execute as a stand alone application or to run perfectly without additional computer code or modification.

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. An identifier element for identifying an origin of a template nucleic acid molecule, comprising:
a nucleic acid element comprising a sequence composition that enables detection of an introduced error in sequence data generated from the nucleic acid element and correction of the introduced error, wherein the nucleic acid element is constructed to couple with the end of a template nucleic acid molecule and identifies an origin of the template nucleic acid molecule.

2. The identifier element of claim 1, wherein:
the sequence composition enables detection of up to three of the introduced errors and correction for up to two of the introduced errors.

3. The identifier element of claim 1, wherein:
The sequence composition comprises 10 sequence positions.

4. The identifier element of claim 1, wherein:
the introduced error is selected from the group consisting of an insertion error, a deletion error, and a substitution error.

5. The identifier element of claim 1, wherein:
the sequence composition comprises a design based upon a set of parameters selected from the group consisting of minimum sequence length, minimum number of flow cycles, sequence distinctiveness, and monomer repeats.

6. The identifier element of claim 1, wherein:
the sequence composition comprises a design based upon a set of parameters selected from the group consisting of melting temperature, Gibbs free energy, hairpin formation, and dimer formation.

7. The identifier element of claim 1, wherein:
the nucleic acid element is incorporated into an adaptor comprising a primer element, wherein the adaptor couples with the end of the template nucleic acid molecule.

8. The identifier element of claim 7, wherein:
the nucleic acid element is in a known position relative to the primer element.

9. The identifier element of claim 7, wherein:
the primer element is selected from the group consisting of an amplification primer, a sequencing primer, or a bipartite amplification-sequencing primer.

10. The identifier element of claim 7, wherein:
the adaptor comprises a quality control element.

11. The identifier element of claim 7, wherein:
the nucleic acid element is in a known position relative to the quality control element.

12. The identifier element of claim 1, wherein:
the origin of the template nucleic acid molecule comprises an experimental sample or diagnostic sample.

13. The identifier element of claim 1, wherein:
the nucleic acid element belongs to a set comprising a plurality of compatible nucleic acid elements each comprising a distinctive sequence composition, wherein the detection of the introduced error is relative to the sequence composition of the compatible nucleic acid elements of the set.

14. The identifier element of claim 13, wherein:
the set comprises 14 of the compatible nucleic acid elements.

15. A method for identifying an origin of a template nucleic acid molecule, comprising the steps of:
identifying a first identifier sequence from sequence data generated from a template nucleic acid molecule;
detecting an introduced error in the first identifier sequence;
correcting the introduced error in the first identifier sequence;
associating the corrected first identifier sequence with a first identifier element coupled to the template molecule; and
identifying an origin of the template molecule using the association of the corrected first identifier sequence with the first identifier element.

16. The method of claim 15, further comprising:
sequencing a template nucleic acid molecule to generate the sequence data.

17. The method of claim 15, wherein:
the template nucleic acid molecule is included in a multiplex sample comprising a plurality of template molecules from a plurality of different origins.

18. The method of claim 15, further comprising:
detecting up to three of the introduced errors in the first identifier sequence; and
correcting up to two of the introduced errors in the first identifier sequence.

19. The method of claim 15, wherein:
the introduced error is selected from the group consisting of an insertion error, a deletion error, and a substitution error.

20. The method of claim 15, wherein the step of detecting comprises:
measuring one or more characteristics of sequence composition in one or more sequence regions that flank the identifier sequence; and
detecting the introduced error using one or more assumptions derived from the measured characteristics.

21. The method of claim 15, wherein:
the first identifier element is incorporated into an adaptor comprising a primer element, wherein the adaptor is coupled to the template nucleic acid molecule.

22. The method of claim 21, wherein:
the first identifier element is in a known position relative to the primer element.

23. The method of claim 21, wherein:
the primer element is selected from the group consisting of an amplification primer, a sequencing primer, or a bipartite amplification-sequencing primer.

24. The method of claim 21, wherein:
the adaptor comprises a quality control element.

25. The method of claim 21, wherein:
the first identifier element is in a known position relative to the quality control element.

26. The method of claim 15, wherein:
the origin of the template nucleic acid molecule comprises an experimental sample or diagnostic sample.

27. The method of claim 15, further comprising the steps of:
identifying a second identifier sequence from the sequence data generated from the template nucleic acid molecule;
detecting an introduced error in the second identifier sequence;
correcting the introduced error in the second identifier sequence;
associating the corrected second identifier sequence with a second identifier element coupled with the template nucleic acid molecule; and
identifying an origin of the template nucleic acid molecule using the association of the corrected second identifier sequence with the second identifier element combinatorially with the association of the corrected first identifier sequence with the first identifier element.

28. The method of claim 27, further comprising:
detecting up to three of the introduced errors in the second identifier sequence; and
correcting up to two of the introduced errors in the second identifier sequence.

29. The method of claim 15, wherein:
the introduced error is selected from the group consisting of an insertion error, a deletion error, and a substitution error.

30. The method of claim 15, wherein:
the first identifier belongs to at least one set of compatible identifiers of a plurality of sets of identifiers.

31. The method of claim 15, wherein:
the set of compatible identifiers comprise 14 identifiers that enable the detection and the correction of the introduced error.

32. A kit for identifying an origin of a template nucleic acid molecule comprising:
a set of nucleic acid elements each comprising a distinctive sequence composition that enables detection of an introduced error in sequence data generated from each nucleic acid element and correction of the introduced error, wherein each of the nucleic acid elements is constructed to couple with the end of a template nucleic acid molecule and identifies the origin of the template nucleic acid molecule.

34. The kit of claim 32, wherein:
the distinctive sequence composition enables detection of up to three of the introduced errors and correction for up to two of the introduced errors.

35. The kit of claim 32, wherein:
the introduced error is selected from the group consisting of an insertion error, a deletion error, and a substitution error.

36. The kit of claim 32, wherein:
each nucleic acid element is incorporated into an adaptor comprising a primer element, wherein the adaptor couples with the end of the template nucleic acid molecule.

37. The kit of claim 36, wherein:
the nucleic acid element is in a known position relative to the primer element.

38. The kit of claim 36, wherein:
the primer element is selected from the group consisting of an amplification primer, a sequencing primer, or a bipartite amplification-sequencing primer.

39. The kit of claim 36, wherein:
the adaptor comprises a quality control element.

40. The kit of claim 36, wherein:
the nucleic acid element is in a known position relative to the quality control element.

41. The kit of claim 32, wherein:
the detection of the introduced error in each of the nucleic acid elements is relative to the distinctive sequence composition of the other nucleic acid elements of the set.

42. The kit of claim 41, wherein:
the set comprises 14 of the nucleic acid elements.

43. A computer, comprising executable code stored thereon, wherein the executable code performs a method for identifying an origin of a template nucleic acid molecule, comprising the steps of:
identifying an identifier sequence from sequence data generated from a template nucleic acid molecule;
detecting an introduced error in the identifier sequence;
correcting the introduced error in the identifier sequence;
associating the corrected identifier sequence with an identifier element coupled with the template molecule; and
identifying an origin of the template molecule using the association of the corrected identifier sequence with the identifier element.

44. The method of claim 43, wherein:
the template nucleic acid molecule is included in a multiplex sample comprising a plurality of template molecules from a plurality of different origins.

45. The method of claim 43, further comprising:
detecting up to three of the introduced errors in the first identifier sequence; and
correcting up to two of the introduced errors in the first identifier sequence.

46. The method of claim 43, wherein:
the introduced error is selected from the group consisting of an insertion error, a deletion error, and a substitution error.

48. The method of claim 43, wherein the step of identifying further comprises:
determining a position for the identifier sequence using a known positional relationship of one or more elements in the sequence data.

49. The method of claim 48, wherein:
the one or more elements include a primer sequence.

50. The method of claim 43, wherein the step of detecting further comprises:
measuring one or more characteristics of sequence composition in one or more sequence regions that flank the identifier sequence; and
detecting the introduced error using one or more assumptions derived from the measured characteristics.

51. The method of claim 43, further comprising:
identifying a second identifier sequence from the sequence data generated from the template nucleic acid molecule;
detecting an introduced error in the second identifier sequence;
correcting the introduced error in the second identifier sequence;
associating the corrected second identifier sequence with a second identifier element coupled with the template molecule; and
identifying an origin of the template molecule using the association of the corrected second identifier sequence with the second identifier element combinatorially with the association of the corrected first identifier sequence with the first identifier element.


SEQUENCE LISTING (1098 sequences)

SEQ ID NO: 1 - DNA, 5 bp, Artificial
  Oligonucleotide
actga

SEQ ID NO: 2 - DNA, 5 bp, Artificial
  Oligonucleotide
agtga

SEQ ID NO: 3 - DNA, 5 bp, Artificial
  Oligonucleotide
agcga

SEQ ID NO: 4 - DNA, 5 bp, Artificial
  Oligonucleotide
ctacc

SEQ ID NO: 5 - DNA, 5 bp, Artificial
  Oligonucleotide
ctgcc

SEQ ID NO: 6 - DNA, 20 bp, Artificial
  Oligonucleotide
tacgtacgtacgtacgtacg

SEQ ID NO: 7 - DNA, 10 bp, Artificial
  Oligonucleotide
acagagtgtc

SEQ ID NO: 8 - DNA, 10 bp, Artificial
  Oligonucleotide
acgtctgaga

SEQ ID NO: 9 - DNA, 10 bp, Artificial
  Oligonucleotide
agacgcactc

SEQ ID NO: 10 - DNA, 10 bp, Artificial
  Oligonucleotide
atctatctcg

SEQ ID NO: 11 - DNA, 10 bp, Artificial
  Oligonucleotide
cgatacgcgt

SEQ ID NO: 12 - DNA, 10 bp, Artificial
  Oligonucleotide
cgcgcgtgcg

SEQ ID NO: 13 - DNA, 10 bp, Artificial
  Oligonucleotide
cgtagatagc

SEQ ID NO: 14 - DNA, 10 bp, Artificial
  Oligonucleotide
cgtgtctcta

SEQ ID NO: 15 - DNA, 10 bp, Artificial
  Oligonucleotide
ctcacacgac

SEQ ID NO: 16 - DNA, 10 bp, Artificial
  Oligonucleotide
tactcatcgt

SEQ ID NO: 17 - DNA, 10 bp, Artificial
  Oligonucleotide
tagcgataca

SEQ ID NO: 18 - DNA, 10 bp, Artificial
  Oligonucleotide
tatgtagtat

SEQ ID NO: 19 - DNA, 10 bp, Artificial
  Oligonucleotide
tctgcgactg

SEQ ID NO: 20 - DNA, 10 bp, Artificial
  Oligonucleotide
tgacagtcag

SEQ ID NO: 21 - DNA, 10 bp, Artificial
  Oligonucleotide
actagcgaga

SEQ ID NO: 22 - DNA, 10 bp, Artificial
  Oligonucleotide
agacgatata

SEQ ID NO: 23 - DNA, 10 bp, Artificial
  Oligonucleotide
atctgacgtc

SEQ ID NO: 24 - DNA, 10 bp, Artificial
  Oligonucleotide
atgtctagcg

SEQ ID NO: 25 - DNA, 10 bp, Artificial
  Oligonucleotide
cgatacgcgt

SEQ ID NO: 26 - DNA, 10 bp, Artificial
  Oligonucleotide
cgcgcgtgcg

SEQ ID NO: 27 - DNA, 10 bp, Artificial
  Oligonucleotide
cgtcacagac

SEQ ID NO: 28 - DNA, 10 bp, Artificial
  Oligonucleotide
cgtgtctcta

SEQ ID NO: 29 - DNA, 10 bp, Artificial
  Oligonucleotide
tactcagatg

SEQ ID NO: 30 - DNA, 10 bp, Artificial
  Oligonucleotide
tagcactctc

SEQ ID NO: 31 - DNA, 10 bp, Artificial
  Oligonucleotide
tatatacaca

SEQ ID NO: 32 - DNA, 10 bp, Artificial
  Oligonucleotide
tcatcgtcag

SEQ ID NO: 33 - DNA, 10 bp, Artificial
  Oligonucleotide
tgagtgcgac

SEQ ID NO: 34 - DNA, 10 bp, Artificial
  Oligonucleotide
tgtgagtagt

SEQ ID NO: 35 - DNA, 10 bp, Artificial
  Oligonucleotide
acactctgac

SEQ ID NO: 36 - DNA, 10 bp, Artificial
  Oligonucleotide
agagagactg

SEQ ID NO: 37 - DNA, 10 bp, Artificial
  Oligonucleotide
atacgtatct

SEQ ID NO: 38 - DNA, 10 bp, Artificial
  Oligonucleotide
atcgtcgaga

SEQ ID NO: 39 - DNA, 10 bp, Artificial
  Oligonucleotide
cacagtagta

SEQ ID NO: 40 - DNA, 10 bp, Artificial
  Oligonucleotide
cgatacgcgt

SEQ ID NO: 41 - DNA, 10 bp, Artificial
  Oligonucleotide
cgcgcgtgcg

SEQ ID NO: 42 - DNA, 10 bp, Artificial
  Oligonucleotide
cgtgtctcta

SEQ ID NO: 43 - DNA, 10 bp, Artificial
  Oligonucleotide
ctgtagatcg

SEQ ID NO: 44 - DNA, 10 bp, Artificial
  Oligonucleotide
tactgagcgc

SEQ ID NO: 45 - DNA, 10 bp, Artificial
  Oligonucleotide
tagctcgtat

SEQ ID NO: 46 - DNA, 10 bp, Artificial
  Oligonucleotide
tatatacaca

SEQ ID NO: 47 - DNA, 10 bp, Artificial
  Oligonucleotide
tcgcacactc

SEQ ID NO: 48 - DNA, 10 bp, Artificial
  Oligonucleotide
tctatgtgtg

SEQ ID NO: 49 - DNA, 10 bp, Artificial
  Oligonucleotide
actcgacgta

SEQ ID NO: 50 - DNA, 10 bp, Artificial
  Oligonucleotide
agactgtaga

SEQ ID NO: 51 - DNA, 10 bp, Artificial
  Oligonucleotide
agagagactg

SEQ ID NO: 52 - DNA, 10 bp, Artificial
  Oligonucleotide
atgtctagcg

SEQ ID NO: 53 - DNA, 10 bp, Artificial
  Oligonucleotide
cactactatg

SEQ ID NO: 54 - DNA, 10 bp, Artificial
  Oligonucleotide
cgatacgcgt

SEQ ID NO: 55 - DNA, 10 bp, Artificial
  Oligonucleotide
cgcgcgtgcg

SEQ ID NO: 56 - DNA, 10 bp, Artificial
  Oligonucleotide
cgtcacagac

SEQ ID NO: 57 - DNA, 10 bp, Artificial
  Oligonucleotide
cgtgtctcta

SEQ ID NO: 58 - DNA, 10 bp, Artificial
  Oligonucleotide
tacgagcagc

SEQ ID NO: 59 - DNA, 10 bp, Artificial
  Oligonucleotide
tagcatcgtg

SEQ ID NO: 60 - DNA, 10 bp, Artificial
  Oligonucleotide
tatacactca

SEQ ID NO: 61 - DNA, 10 bp, Artificial
  Oligonucleotide
tctgtgtgac

SEQ ID NO: 62 - DNA, 10 bp, Artificial
  Oligonucleotide
tgtagtacat

SEQ ID NO: 63 - DNA, 10 bp, Artificial
  Oligonucleotide
actcgatctc

SEQ ID NO: 64 - DNA, 10 bp, Artificial
  Oligonucleotide
agactgtaga

SEQ ID NO: 65 - DNA, 10 bp, Artificial
  Oligonucleotide
agagagactg

SEQ ID NO: 66 - DNA, 10 bp, Artificial
  Oligonucleotide
atgtctagcg

SEQ ID NO: 67 - DNA, 10 bp, Artificial
  Oligonucleotide
cactactatg

SEQ ID NO: 68 - DNA, 10 bp, Artificial
  Oligonucleotide
cgatacgcgt

SEQ ID NO: 69 - DNA, 10 bp, Artificial
  Oligonucleotide
cgcgcgtgcg

SEQ ID NO: 70 - DNA, 10 bp, Artificial
  Oligonucleotide
cgtcacagac

SEQ ID NO: 71 - DNA, 10 bp, Artificial
  Oligonucleotide
cgtgtctcta

SEQ ID NO: 72 - DNA, 10 bp, Artificial
  Oligonucleotide
tacgagcagc

SEQ ID NO: 73 - DNA, 10 bp, Artificial
  Oligonucleotide
tagcatcgtg

SEQ ID NO: 74 - DNA, 10 bp, Artificial
  Oligonucleotide
tatacactca

SEQ ID NO: 75 - DNA, 10 bp, Artificial
  Oligonucleotide
tctgtgtgac

SEQ ID NO: 76 - DNA, 10 bp, Artificial
  Oligonucleotide
tgtagtacat

SEQ ID NO: 77 - DNA, 10 bp, Artificial
  Oligonucleotide
acacacgctg

SEQ ID NO: 78 - DNA, 10 bp, Artificial
  Oligonucleotide
acgatcatag

SEQ ID NO: 79 - DNA, 10 bp, Artificial
  Oligonucleotide
agagagacac

SEQ ID NO: 80 - DNA, 10 bp, Artificial
  Oligonucleotide
atactatgac

SEQ ID NO: 81 - DNA, 10 bp, Artificial
  Oligonucleotide
cgcgcgtgcg

SEQ ID NO: 82 - DNA, 10 bp, Artificial
  Oligonucleotide
cgtgtctcta

SEQ ID NO: 83 - DNA, 10 bp, Artificial
  Oligonucleotide
ctacgacagt

SEQ ID NO: 84 - DNA, 10 bp, Artificial
  Oligonucleotide
ctgtagatcg

SEQ ID NO: 85 - DNA, 10 bp, Artificial
  Oligonucleotide
tagcagtcta

SEQ ID NO: 86 - DNA, 10 bp, Artificial
  Oligonucleotide
tagtgcgcgt

SEQ ID NO: 87 - DNA, 10 bp, Artificial
  Oligonucleotide
tctatgtgtg

SEQ ID NO: 88 - DNA, 10 bp, Artificial
  Oligonucleotide
tctcgctcac

SEQ ID NO: 89 - DNA, 10 bp, Artificial
  Oligonucleotide
tgatactact

SEQ ID NO: 90 - DNA, 10 bp, Artificial
  Oligonucleotide
tgtctagaga

SEQ ID NO: 91 - DNA, 10 bp, Artificial
  Oligonucleotide
acagtctacg

SEQ ID NO: 92 - DNA, 10 bp, Artificial
  Oligonucleotide
agacgcactc

SEQ ID NO: 93 - DNA, 10 bp, Artificial
  Oligonucleotide
agctacatag

SEQ ID NO: 94 - DNA, 10 bp, Artificial
  Oligonucleotide
agtctgtgac

SEQ ID NO: 95 - DNA, 10 bp, Artificial
  Oligonucleotide
atagagtgta

SEQ ID NO: 96 - DNA, 10 bp, Artificial
  Oligonucleotide
cgcgcgtgcg

SEQ ID NO: 97 - DNA, 10 bp, Artificial
  Oligonucleotide
cgtgtctcta

SEQ ID NO: 98 - DNA, 10 bp, Artificial
  Oligonucleotide
ctactagctg

SEQ ID NO: 99 - DNA, 10 bp, Artificial
  Oligonucleotide
ctgagacgag

SEQ ID NO: 100 - DNA, 10 bp, Artificial
  Oligonucleotide
tagtgcgcgt

SEQ ID NO: 101 - DNA, 10 bp, Artificial
  Oligonucleotide
tcacgtatga

SEQ ID NO: 102 - DNA, 10 bp, Artificial
  Oligonucleotide
tcgtcagatc

SEQ ID NO: 103 - DNA, 10 bp, Artificial
  Oligonucleotide
tctatacaca

SEQ ID NO: 104 - DNA, 10 bp, Artificial
  Oligonucleotide
tgatagtcgc

SEQ ID NO: 105 - DNA, 10 bp, Artificial
  Oligonucleotide
acgatcgcgt

SEQ ID NO: 106 - DNA, 10 bp, Artificial
  Oligonucleotide
actctatgtg

SEQ ID NO: 107 - DNA, 10 bp, Artificial
  Oligonucleotide
agcacactag

SEQ ID NO: 108 - DNA, 10 bp, Artificial
  Oligonucleotide
atacagagtc

SEQ ID NO: 109 - DNA, 10 bp, Artificial
  Oligonucleotide
atatctcacg

SEQ ID NO: 110 - DNA, 10 bp, Artificial
  Oligonucleotide
cagtatacgc

SEQ ID NO: 111 - DNA, 10 bp, Artificial
  Oligonucleotide
cgcgcgtgcg

SEQ ID NO: 112 - DNA, 10 bp, Artificial
  Oligonucleotide
cgtgtctcta

SEQ ID NO: 113 - DNA, 10 bp, Artificial
  Oligonucleotide
ctacgtacat

SEQ ID NO: 114 - DNA, 10 bp, Artificial
  Oligonucleotide
tatacgctga

SEQ ID NO: 115 - DNA, 10 bp, Artificial
  Oligonucleotide
tcgacatctc

SEQ ID NO: 116 - DNA, 10 bp, Artificial
  Oligonucleotide
tcgctagaca

SEQ ID NO: 117 - DNA, 10 bp, Artificial
  Oligonucleotide
tgagtgcgac

SEQ ID NO: 118 - DNA, 10 bp, Artificial
  Oligonucleotide
tgtgagtagt

SEQ ID NO: 119 - DNA, 10 bp, Artificial
  Oligonucleotide
acacgctcac

SEQ ID NO: 120 - DNA, 10 bp, Artificial
  Oligonucleotide
acgatcgcgt

SEQ ID NO: 121 - DNA, 10 bp, Artificial
  Oligonucleotide
agacgagaga

SEQ ID NO: 122 - DNA, 10 bp, Artificial
  Oligonucleotide
agctgtatac

SEQ ID NO: 123 - DNA, 10 bp, Artificial
  Oligonucleotide
atagtgatcg

SEQ ID NO: 124 - DNA, 10 bp, Artificial
  Oligonucleotide
cactacactg

SEQ ID NO: 125 - DNA, 10 bp, Artificial
  Oligonucleotide
cgcgcgtgcg

SEQ ID NO: 126 - DNA, 10 bp, Artificial
  Oligonucleotide
cgtagacgac

SEQ ID NO: 127 - DNA, 10 bp, Artificial
  Oligonucleotide
cgtgtctcta

SEQ ID NO: 128 - DNA, 10 bp, Artificial
  Oligonucleotide
tacatatgag

SEQ ID NO: 129 - DNA, 10 bp, Artificial
  Oligonucleotide
tctctcgaca

SEQ ID NO: 130 - DNA, 10 bp, Artificial
  Oligonucleotide
tctgagctag

SEQ ID NO: 131 - DNA, 10 bp, Artificial
  Oligonucleotide
tgagatactc

SEQ ID NO: 132 - DNA, 10 bp, Artificial
  Oligonucleotide
tgtactgtgt

SEQ ID NO: 133 - DNA, 10 bp, Artificial
  Oligonucleotide
acatcgtctg

SEQ ID NO: 134 - DNA, 10 bp, Artificial
  Oligonucleotide
acgctgtagt

SEQ ID NO: 135 - DNA, 10 bp, Artificial
  Oligonucleotide
agagagacac

SEQ ID NO: 136 - DNA, 10 bp, Artificial
  Oligonucleotide
agtcagcgta

SEQ ID NO: 137 - DNA, 10 bp, Artificial
  Oligonucleotide
atacgcacga

SEQ ID NO: 138 - DNA, 10 bp, Artificial
  Oligonucleotide
catgtagacg

SEQ ID NO: 139 - DNA, 10 bp, Artificial
  Oligonucleotide
cgcgcgtgcg

SEQ ID NO: 140 - DNA, 10 bp, Artificial
  Oligonucleotide
cgtgtctcta

SEQ ID NO: 141 - DNA, 10 bp, Artificial
  Oligonucleotide
ctacatgtac

SEQ ID NO: 142 - DNA, 10 bp, Artificial
  Oligonucleotide
tactactgct

SEQ ID NO: 143 - DNA, 10 bp, Artificial
  Oligonucleotide
tagagatgtg

SEQ ID NO: 144 - DNA, 10 bp, Artificial
  Oligonucleotide
tcagactaga

SEQ ID NO: 145 - DNA, 10 bp, Artificial
  Oligonucleotide
tgtatcgagc

SEQ ID NO: 146 - DNA, 10 bp, Artificial
  Oligonucleotide
tgtcgtacat

SEQ ID NO: 147 - DNA, 10 bp, Artificial
  Oligonucleotide
acatcgtctg

SEQ ID NO: 148 - DNA, 10 bp, Artificial
  Oligonucleotide
acgctgtagt

SEQ ID NO: 149 - DNA, 10 bp, Artificial
  Oligonucleotide
agagagacac

SEQ ID NO: 150 - DNA, 10 bp, Artificial
  Oligonucleotide
agtcagcgta

SEQ ID NO: 151 - DNA, 10 bp, Artificial
  Oligonucleotide
atacgcacga

SEQ ID NO: 152 - DNA, 10 bp, Artificial
  Oligonucleotide
catgtagacg

SEQ ID NO: 153 - DNA, 10 bp, Artificial
  Oligonucleotide
cgcgcgtgcg

SEQ ID NO: 154 - DNA, 10 bp, Artificial
  Oligonucleotide
cgtgtctcta

SEQ ID NO: 155 - DNA, 10 bp, Artificial
  Oligonucleotide
ctacatgtac

SEQ ID NO: 156 - DNA, 10 bp, Artificial
  Oligonucleotide
tactactgct

SEQ ID NO: 157 - DNA, 10 bp, Artificial
  Oligonucleotide
tagagatgtg

SEQ ID NO: 158 - DNA, 10 bp, Artificial
  Oligonucleotide
tcagactaga

SEQ ID NO: 159 - DNA, 10 bp, Artificial
  Oligonucleotide
tcgcacactc

SEQ ID NO: 160 - DNA, 10 bp, Artificial
  Oligonucleotide
tgtatcgagc

SEQ ID NO: 161 - DNA, 10 bp, Artificial
  Oligonucleotide
acacgctcac

SEQ ID NO: 162 - DNA, 10 bp, Artificial
  Oligonucleotide
acgctgtagt

SEQ ID NO: 163 - DNA, 10 bp, Artificial
  Oligonucleotide
agtcatcgtc

SEQ ID NO: 164 - DNA, 10 bp, Artificial
  Oligonucleotide
agtgagacag

SEQ ID NO: 165 - DNA, 10 bp, Artificial
  Oligonucleotide
atagagtgta

SEQ ID NO: 166 - DNA, 10 bp, Artificial
  Oligonucleotide
cacatacgag

SEQ ID NO: 167 - DNA, 10 bp, Artificial
  Oligonucleotide
cgcgcgtgcg

SEQ ID NO: 168 - DNA, 10 bp, Artificial
  Oligonucleotide
cgtgtctcta

SEQ ID NO: 169 - DNA, 10 bp, Artificial
  Oligonucleotide
ctctagactc

SEQ ID NO: 170 - DNA, 10 bp, Artificial
  Oligonucleotide
tatactcaga

SEQ ID NO: 171 - DNA, 10 bp, Artificial
  Oligonucleotide
tatcgtatcg

SEQ ID NO: 172 - DNA, 10 bp, Artificial
  Oligonucleotide
tcgtacatat

SEQ ID NO: 173 - DNA, 10 bp, Artificial
  Oligonucleotide
tctgtgtgac

SEQ ID NO: 174 - DNA, 10 bp, Artificial
  Oligonucleotide
tgacgacgct

SEQ ID NO: 175 - DNA, 10 bp, Artificial
  Oligonucleotide
acatcgtctg

SEQ ID NO: 176 - DNA, 10 bp, Artificial
  Oligonucleotide
acgcacgata

SEQ ID NO: 177 - DNA, 10 bp, Artificial
  Oligonucleotide
agagtagcgt

SEQ ID NO: 178 - DNA, 10 bp, Artificial
  Oligonucleotide
agtacacact

SEQ ID NO: 179 - DNA, 10 bp, Artificial
  Oligonucleotide
agtctgtgac

SEQ ID NO: 180 - DNA, 10 bp, Artificial
  Oligonucleotide
cactactagc

SEQ ID NO: 181 - DNA, 10 bp, Artificial
  Oligonucleotide
cgcgcgtgcg

SEQ ID NO: 182 - DNA, 10 bp, Artificial
  Oligonucleotide
cgtgtctcta

SEQ ID NO: 183 - DNA, 10 bp, Artificial
  Oligonucleotide
ctacagtgta

SEQ ID NO: 184 - DNA, 10 bp, Artificial
  Oligonucleotide
ctgtagatcg

SEQ ID NO: 185 - DNA, 10 bp, Artificial
  Oligonucleotide
tatcgcgagt

SEQ ID NO: 186 - DNA, 10 bp, Artificial
  Oligonucleotide
tcgacagcag

SEQ ID NO: 187 - DNA, 10 bp, Artificial
  Oligonucleotide
tctcatacga

SEQ ID NO: 188 - DNA, 10 bp, Artificial
  Oligonucleotide
tgagatatac

SEQ ID NO: 189 - DNA, 10 bp, Artificial
  Oligonucleotide
acagctcgac

SEQ ID NO: 190 - DNA, 10 bp, Artificial
  Oligonucleotide
actcgataca

SEQ ID NO: 191 - DNA, 10 bp, Artificial
  Oligonucleotide
agatagacta

SEQ ID NO: 192 - DNA, 10 bp, Artificial
  Oligonucleotide
agcgcgtgtg

SEQ ID NO: 193 - DNA, 10 bp, Artificial
  Oligonucleotide
atgtctagcg

SEQ ID NO: 194 - DNA, 10 bp, Artificial
  Oligonucleotide
cacgtagtat

SEQ ID NO: 195 - DNA, 10 bp, Artificial
  Oligonucleotide
cgacacgcag

SEQ ID NO: 196 - DNA, 10 bp, Artificial
  Oligonucleotide
cgtgagagac

SEQ ID NO: 197 - DNA, 10 bp, Artificial
  Oligonucleotide
cgtgtctcta

SEQ ID NO: 198 - DNA, 10 bp, Artificial
  Oligonucleotide
tactatcgct

SEQ ID NO: 199 - DNA, 10 bp, Artificial
  Oligonucleotide
tagtgcacag

SEQ ID NO: 200 - DNA, 10 bp, Artificial
  Oligonucleotide
tcgcagacgt

SEQ ID NO: 201 - DNA, 10 bp, Artificial
  Oligonucleotide
tctactgtga

SEQ ID NO: 202 - DNA, 10 bp, Artificial
  Oligonucleotide
tgacgtcatc

SEQ ID NO: 203 - DNA, 10 bp, Artificial
  Oligonucleotide
acagacatag

SEQ ID NO: 204 - DNA, 10 bp, Artificial
  Oligonucleotide
acgcgcgtgt

SEQ ID NO: 205 - DNA, 10 bp, Artificial
  Oligonucleotide
agacgcactc

SEQ ID NO: 206 - DNA, 10 bp, Artificial
  Oligonucleotide
agctatcgag

SEQ ID NO: 207 - DNA, 10 bp, Artificial
  Oligonucleotide
atatgtgtac

SEQ ID NO: 208 - DNA, 10 bp, Artificial
  Oligonucleotide
atgtagacgt

SEQ ID NO: 209 - DNA, 10 bp, Artificial
  Oligonucleotide
cactcgtcac

SEQ ID NO: 210 - DNA, 10 bp, Artificial
  Oligonucleotide
cgtgagagac

SEQ ID NO: 211 - DNA, 10 bp, Artificial
  Oligonucleotide
cgtgtctcta

SEQ ID NO: 212 - DNA, 10 bp, Artificial
  Oligonucleotide
ctcactagtg

SEQ ID NO: 213 - DNA, 10 bp, Artificial
  Oligonucleotide
tactatatct

SEQ ID NO: 214 - DNA, 10 bp, Artificial
  Oligonucleotide
tatactcaga

SEQ ID NO: 215 - DNA, 10 bp, Artificial
  Oligonucleotide
tcgcagtaca

SEQ ID NO: 216 - DNA, 10 bp, Artificial
  Oligonucleotide
tgagtgcgcg

SEQ ID NO: 217 - DNA, 10 bp, Artificial
  Oligonucleotide
acagactgtc

SEQ ID NO: 218 - DNA, 10 bp, Artificial
  Oligonucleotide
agcgcgtaca

SEQ ID NO: 219 - DNA, 10 bp, Artificial
  Oligonucleotide
agtcgagctg

SEQ ID NO: 220 - DNA, 10 bp, Artificial
  Oligonucleotide
atactagagc

SEQ ID NO: 221 - DNA, 10 bp, Artificial
  Oligonucleotide
atcgtcacgt

SEQ ID NO: 222 - DNA, 10 bp, Artificial
  Oligonucleotide
cgagcacgcg

SEQ ID NO: 223 - DNA, 10 bp, Artificial
  Oligonucleotide
cgtgagagac

SEQ ID NO: 224 - DNA, 10 bp, Artificial
  Oligonucleotide
cgtgtctcta

SEQ ID NO: 225 - DNA, 10 bp, Artificial
  Oligonucleotide
ctcatcgtag

SEQ ID NO: 226 - DNA, 10 bp, Artificial
  Oligonucleotide
tacactcgct

SEQ ID NO: 227 - DNA, 10 bp, Artificial
  Oligonucleotide
tatgtagtat

SEQ ID NO: 228 - DNA, 10 bp, Artificial
  Oligonucleotide
tcgctgtgtg

SEQ ID NO: 229 - DNA, 10 bp, Artificial
  Oligonucleotide
tctatacaca

SEQ ID NO: 230 - DNA, 10 bp, Artificial
  Oligonucleotide
tgacgtcatc

SEQ ID NO: 231 - DNA, 10 bp, Artificial
  Oligonucleotide
actgtgatcg

SEQ ID NO: 232 - DNA, 10 bp, Artificial
  Oligonucleotide
agcgcgtaca

SEQ ID NO: 233 - DNA, 10 bp, Artificial
  Oligonucleotide
agtactgtgt

SEQ ID NO: 234 - DNA, 10 bp, Artificial
  Oligonucleotide
atactcacac

SEQ ID NO: 235 - DNA, 10 bp, Artificial
  Oligonucleotide
cacagtagta

SEQ ID NO: 236 - DNA, 10 bp, Artificial
  Oligonucleotide
cgagcacgcg

SEQ ID NO: 237 - DNA, 10 bp, Artificial
  Oligonucleotide
cgtgagagac

SEQ ID NO: 238 - DNA, 10 bp, Artificial
  Oligonucleotide
cgtgtctcta

SEQ ID NO: 239 - DNA, 10 bp, Artificial
  Oligonucleotide
ctcatctacg

SEQ ID NO: 240 - DNA, 10 bp, Artificial
  Oligonucleotide
tcgctatgag

SEQ ID NO: 241 - DNA, 10 bp, Artificial
  Oligonucleotide
tctatacata

SEQ ID NO: 242 - DNA, 10 bp, Artificial
  Oligonucleotide
tctcgcgcgt

SEQ ID NO: 243 - DNA, 10 bp, Artificial
  Oligonucleotide
tgacgactat

SEQ ID NO: 244 - DNA, 10 bp, Artificial
  Oligonucleotide
tgatagtcgc

SEQ ID NO: 245 - DNA, 10 bp, Artificial
  Oligonucleotide
actgtgatcg

SEQ ID NO: 246 - DNA, 10 bp, Artificial
  Oligonucleotide
agcgcgtaca

SEQ ID NO: 247 - DNA, 10 bp, Artificial
  Oligonucleotide
agtactgtgt

SEQ ID NO: 248 - DNA, 10 bp, Artificial
  Oligonucleotide
cacagtagta

SEQ ID NO: 249 - DNA, 10 bp, Artificial
  Oligonucleotide
cgagcacgcg

SEQ ID NO: 250 - DNA, 10 bp, Artificial
  Oligonucleotide
cgtgagagac

SEQ ID NO: 251 - DNA, 10 bp, Artificial
  Oligonucleotide
cgtgtctcta

SEQ ID NO: 252 - DNA, 10 bp, Artificial
  Oligonucleotide
ctcatctacg

SEQ ID NO: 253 - DNA, 10 bp, Artificial
  Oligonucleotide
tagctcactc

SEQ ID NO: 254 - DNA, 10 bp, Artificial
  Oligonucleotide
tcgctatgag

SEQ ID NO: 255 - DNA, 10 bp, Artificial
  Oligonucleotide
tctatacata

SEQ ID NO: 256 - DNA, 10 bp, Artificial
  Oligonucleotide
tctcgcgcgt

SEQ ID NO: 257 - DNA, 10 bp, Artificial
  Oligonucleotide
tgacgactat

SEQ ID NO: 258 - DNA, 10 bp, Artificial
  Oligonucleotide
tgatagtcgc

SEQ ID NO: 259 - DNA, 10 bp, Artificial
  Oligonucleotide
actgtgatcg

SEQ ID NO: 260 - DNA, 10 bp, Artificial
  Oligonucleotide
agcgcgtaca

SEQ ID NO: 261 - DNA, 10 bp, Artificial
  Oligonucleotide
agtatacagt

SEQ ID NO: 262 - DNA, 10 bp, Artificial
  Oligonucleotide
catacgtgtg

SEQ ID NO: 263 - DNA, 10 bp, Artificial
  Oligonucleotide
cgagcacgcg

SEQ ID NO: 264 - DNA, 10 bp, Artificial
  Oligonucleotide
cgtgagagac

SEQ ID NO: 265 - DNA, 10 bp, Artificial
  Oligonucleotide
cgtgtctcta

SEQ ID NO: 266 - DNA, 10 bp, Artificial
  Oligonucleotide
ctcatctacg

SEQ ID NO: 267 - DNA, 10 bp, Artificial
  Oligonucleotide
tacatagata

SEQ ID NO: 268 - DNA, 10 bp, Artificial
  Oligonucleotide
tagctcactc

SEQ ID NO: 269 - DNA, 10 bp, Artificial
  Oligonucleotide
tcgctatgag

SEQ ID NO: 270 - DNA, 10 bp, Artificial
  Oligonucleotide
tctcgcgcgt

SEQ ID NO: 271 - DNA, 10 bp, Artificial
  Oligonucleotide
tgacgactat

SEQ ID NO: 272 - DNA, 10 bp, Artificial
  Oligonucleotide
tgatagtcgc

SEQ ID NO: 273 - DNA, 10 bp, Artificial
  Oligonucleotide
actactcaga

SEQ ID NO: 274 - DNA, 10 bp, Artificial
  Oligonucleotide
actgtgatcg

SEQ ID NO: 275 - DNA, 10 bp, Artificial
  Oligonucleotide
agcgcgtaca

SEQ ID NO: 276 - DNA, 10 bp, Artificial
  Oligonucleotide
catacgtgtg

SEQ ID NO: 277 - DNA, 10 bp, Artificial
  Oligonucleotide
cgagcacgcg

SEQ ID NO: 278 - DNA, 10 bp, Artificial
  Oligonucleotide
cgtgagagac

SEQ ID NO: 279 - DNA, 10 bp, Artificial
  Oligonucleotide
cgtgtctcta

SEQ ID NO: 280 - DNA, 10 bp, Artificial
  Oligonucleotide
ctcatctacg

SEQ ID NO: 281 - DNA, 10 bp, Artificial
  Oligonucleotide
tacatagata

SEQ ID NO: 282 - DNA, 10 bp, Artificial
  Oligonucleotide
tagctcactc

SEQ ID NO: 283 - DNA, 10 bp, Artificial
  Oligonucleotide
tcgctatgag

SEQ ID NO: 284 - DNA, 10 bp, Artificial
  Oligonucleotide
tctcgcgcgt

SEQ ID NO: 285 - DNA, 10 bp, Artificial
  Oligonucleotide
tgacgactat

SEQ ID NO: 286 - DNA, 10 bp, Artificial
  Oligonucleotide
tgatagtcgc

SEQ ID NO: 287 - DNA, 10 bp, Artificial
  Oligonucleotide
acagactgtc

SEQ ID NO: 288 - DNA, 10 bp, Artificial
  Oligonucleotide
actcgcgctg

SEQ ID NO: 289 - DNA, 10 bp, Artificial
  Oligonucleotide
agcgcgtaca

SEQ ID NO: 290 - DNA, 10 bp, Artificial
  Oligonucleotide
agtatacatg

SEQ ID NO: 291 - DNA, 10 bp, Artificial
  Oligonucleotide
atacagtcga

SEQ ID NO: 292 - DNA, 10 bp, Artificial
  Oligonucleotide
cacgtagtat

SEQ ID NO: 293 - DNA, 10 bp, Artificial
  Oligonucleotide
cgagcacgcg

SEQ ID NO: 294 - DNA, 10 bp, Artificial
  Oligonucleotide
cgtgagagac

SEQ ID NO: 295 - DNA, 10 bp, Artificial
  Oligonucleotide
cgtgtctcta

SEQ ID NO: 296 - DNA, 10 bp, Artificial
  Oligonucleotide
ctcatctacg

SEQ ID NO: 297 - DNA, 10 bp, Artificial
  Oligonucleotide
tagctcactc

SEQ ID NO: 298 - DNA, 10 bp, Artificial
  Oligonucleotide
tatgtgtgcg

SEQ ID NO: 299 - DNA, 10 bp, Artificial
  Oligonucleotide
tcgatcgtga

SEQ ID NO: 300 - DNA, 10 bp, Artificial
  Oligonucleotide
tgactgatag

SEQ ID NO: 301 - DNA, 10 bp, Artificial
  Oligonucleotide
acagacatag

SEQ ID NO: 302 - DNA, 10 bp, Artificial
  Oligonucleotide
actcgcgctg

SEQ ID NO: 303 - DNA, 10 bp, Artificial
  Oligonucleotide
agcgcgtaca

SEQ ID NO: 304 - DNA, 10 bp, Artificial
  Oligonucleotide
agtatacagt

SEQ ID NO: 305 - DNA, 10 bp, Artificial
  Oligonucleotide
atacagtcga

SEQ ID NO: 306 - DNA, 10 bp, Artificial
  Oligonucleotide
cacgtagtat

SEQ ID NO: 307 - DNA, 10 bp, Artificial
  Oligonucleotide
cgagcacgcg

SEQ ID NO: 308 - DNA, 10 bp, Artificial
  Oligonucleotide
cgtgagagac

SEQ ID NO: 309 - DNA, 10 bp, Artificial
  Oligonucleotide
cgtgtctcta

SEQ ID NO: 310 - DNA, 10 bp, Artificial
  Oligonucleotide
ctcatctacg

SEQ ID NO: 311 - DNA, 10 bp, Artificial
  Oligonucleotide
tagatatctc

SEQ ID NO: 312 - DNA, 10 bp, Artificial
  Oligonucleotide
tatgtgtgcg

SEQ ID NO: 313 - DNA, 10 bp, Artificial
  Oligonucleotide
tcgatcgtga

SEQ ID NO: 314 - DNA, 10 bp, Artificial
  Oligonucleotide
tgtactgatg

SEQ ID NO: 315 - DNA, 10 bp, Artificial
  Oligonucleotide
acagacatag

SEQ ID NO: 316 - DNA, 10 bp, Artificial
  Oligonucleotide
actcgcgctg

SEQ ID NO: 317 - DNA, 10 bp, Artificial
  Oligonucleotide
agcgcgtaca

SEQ ID NO: 318 - DNA, 10 bp, Artificial
  Oligonucleotide
agtatacagt

SEQ ID NO: 319 - DNA, 10 bp, Artificial
  Oligonucleotide
atacagtcga

SEQ ID NO: 320 - DNA, 10 bp, Artificial
  Oligonucleotide
cacgtagtat

SEQ ID NO: 321 - DNA, 10 bp, Artificial
  Oligonucleotide
cgagcacgcg

SEQ ID NO: 322 - DNA, 10 bp, Artificial
  Oligonucleotide
cgtgagagac

SEQ ID NO: 323 - DNA, 10 bp, Artificial
  Oligonucleotide
cgtgtctcta

SEQ ID NO: 324 - DNA, 10 bp, Artificial
  Oligonucleotide
ctcatctacg

SEQ ID NO: 325 - DNA, 10 bp, Artificial
  Oligonucleotide
tagctagctc

SEQ ID NO: 326 - DNA, 10 bp, Artificial
  Oligonucleotide
tatgtgtgcg

SEQ ID NO: 327 - DNA, 10 bp, Artificial
  Oligonucleotide
tcgatcgtga

SEQ ID NO: 328 - DNA, 10 bp, Artificial
  Oligonucleotide
tgtactgatg

SEQ ID NO: 329 - DNA, 10 bp, Artificial
  Oligonucleotide
acagacatag

SEQ ID NO: 330 - DNA, 10 bp, Artificial
  Oligonucleotide
actcgcgctg

SEQ ID NO: 331 - DNA, 10 bp, Artificial
  Oligonucleotide
agcgcgtaca

SEQ ID NO: 332 - DNA, 10 bp, Artificial
  Oligonucleotide
agtatacagt

SEQ ID NO: 333 - DNA, 10 bp, Artificial
  Oligonucleotide
atacagtcga

SEQ ID NO: 334 - DNA, 10 bp, Artificial
  Oligonucleotide
cacgtagtat

SEQ ID NO: 335 - DNA, 10 bp, Artificial
  Oligonucleotide
cgagcacgcg

SEQ ID NO: 336 - DNA, 10 bp, Artificial
  Oligonucleotide
cgtgagagac

SEQ ID NO: 337 - DNA, 10 bp, Artificial
  Oligonucleotide
cgtgtctcta

SEQ ID NO: 338 - DNA, 10 bp, Artificial
  Oligonucleotide
ctcatctacg

SEQ ID NO: 339 - DNA, 10 bp, Artificial
  Oligonucleotide
tagctcactc

SEQ ID NO: 340 - DNA, 10 bp, Artificial
  Oligonucleotide
tatgtgtgcg

SEQ ID NO: 341 - DNA, 10 bp, Artificial
  Oligonucleotide
tcgatcgtga

SEQ ID NO: 342 - DNA, 10 bp, Artificial
  Oligonucleotide
tgtactgatg

SEQ ID NO: 343 - DNA, 10 bp, Artificial
  Oligonucleotide
acagacatag

SEQ ID NO: 344 - DNA, 10 bp, Artificial
  Oligonucleotide
actcgcgctg

SEQ ID NO: 345 - DNA, 10 bp, Artificial
  Oligonucleotide
agcgcgtaca

SEQ ID NO: 346 - DNA, 10 bp, Artificial
  Oligonucleotide
atacagtcga

SEQ ID NO: 347 - DNA, 10 bp, Artificial
  Oligonucleotide
cacgtagtat

SEQ ID NO: 348 - DNA, 10 bp, Artificial
  Oligonucleotide
cgagcacgcg

SEQ ID NO: 349 - DNA, 10 bp, Artificial
  Oligonucleotide
cgtgagagac

SEQ ID NO: 350 - DNA, 10 bp, Artificial
  Oligonucleotide
cgtgtctcta

SEQ ID NO: 351 - DNA, 10 bp, Artificial
  Oligonucleotide
ctcatctacg

SEQ ID NO: 352 - DNA, 10 bp, Artificial
  Oligonucleotide
tagatatctc

SEQ ID NO: 353 - DNA, 10 bp, Artificial
  Oligonucleotide
tagcgacagt

SEQ ID NO: 354 - DNA, 10 bp, Artificial
  Oligonucleotide
tatgtgtgcg

SEQ ID NO: 355 - DNA, 10 bp, Artificial
  Oligonucleotide
tcgatcgtga

SEQ ID NO: 356 - DNA, 10 bp, Artificial
  Oligonucleotide
tgtactgatg

SEQ ID NO: 357 - DNA, 10 bp, Artificial
  Oligonucleotide
actcgcgctg

SEQ ID NO: 358 - DNA, 10 bp, Artificial
  Oligonucleotide
agcgcgtaca

SEQ ID NO: 359 - DNA, 10 bp, Artificial
  Oligonucleotide
atacagtcga

SEQ ID NO: 360 - DNA, 10 bp, Artificial
  Oligonucleotide
atctacacac

SEQ ID NO: 361 - DNA, 10 bp, Artificial
  Oligonucleotide
cacgtagtat

SEQ ID NO: 362 - DNA, 10 bp, Artificial
  Oligonucleotide
cgagcacgcg

SEQ ID NO: 363 - DNA, 10 bp, Artificial
  Oligonucleotide
cgtgagagac

SEQ ID NO: 364 - DNA, 10 bp, Artificial
  Oligonucleotide
cgtgtctcta

SEQ ID NO: 365 - DNA, 10 bp, Artificial
  Oligonucleotide
ctcatctacg

SEQ ID NO: 366 - DNA, 10 bp, Artificial
  Oligonucleotide
tagatatctc

SEQ ID NO: 367 - DNA, 10 bp, Artificial
  Oligonucleotide
tagcgacagt

SEQ ID NO: 368 - DNA, 10 bp, Artificial
  Oligonucleotide
tatgtgtgcg

SEQ ID NO: 369 - DNA, 10 bp, Artificial
  Oligonucleotide
tcgatcgtga

SEQ ID NO: 370 - DNA, 10 bp, Artificial
  Oligonucleotide
tgtactgatg

SEQ ID NO: 371 - DNA, 10 bp, Artificial
  Oligonucleotide
acagacatag

SEQ ID NO: 372 - DNA, 10 bp, Artificial
  Oligonucleotide
actcgcgctg

SEQ ID NO: 373 - DNA, 10 bp, Artificial
  Oligonucleotide
actctatgac

SEQ ID NO: 374 - DNA, 10 bp, Artificial
  Oligonucleotide
agcgcgtaca

SEQ ID NO: 375 - DNA, 10 bp, Artificial
  Oligonucleotide
agtatacagt

SEQ ID NO: 376 - DNA, 10 bp, Artificial
  Oligonucleotide
atacagtcga

SEQ ID NO: 377 - DNA, 10 bp, Artificial
  Oligonucleotide
cacgtagtat

SEQ ID NO: 378 - DNA, 10 bp, Artificial
  Oligonucleotide
cgagcacgcg

SEQ ID NO: 379 - DNA, 10 bp, Artificial
  Oligonucleotide
cgtgagagac

SEQ ID NO: 380 - DNA, 10 bp, Artificial
  Oligonucleotide
cgtgtctcta

SEQ ID NO: 381 - DNA, 10 bp, Artificial
  Oligonucleotide
tagatatctc

SEQ ID NO: 382 - DNA, 10 bp, Artificial
  Oligonucleotide
tatgtgtgcg

SEQ ID NO: 383 - DNA, 10 bp, Artificial
  Oligonucleotide
tcgatcgtga

SEQ ID NO: 384 - DNA, 10 bp, Artificial
  Oligonucleotide
tgtactgatg

SEQ ID NO: 385 - DNA, 10 bp, Artificial
  Oligonucleotide
acagacatag

SEQ ID NO: 386 - DNA, 10 bp, Artificial
  Oligonucleotide
actcgcgctg

SEQ ID NO: 387 - DNA, 10 bp, Artificial
  Oligonucleotide
actctatgac

SEQ ID NO: 388 - DNA, 10 bp, Artificial
  Oligonucleotide
agcgcgtaca

SEQ ID NO: 389 - DNA, 10 bp, Artificial
  Oligonucleotide
agtatacagt

SEQ ID NO: 390 - DNA, 10 bp, Artificial
  Oligonucleotide
atacagtcga

SEQ ID NO: 391 - DNA, 10 bp, Artificial
  Oligonucleotide
cacgtagtat

SEQ ID NO: 392 - DNA, 10 bp, Artificial
  Oligonucleotide
cgagcacgcg

SEQ ID NO: 393 - DNA, 10 bp, Artificial
  Oligonucleotide
cgtgagagac

SEQ ID NO: 394 - DNA, 10 bp, Artificial
  Oligonucleotide
cgtgtctcta

SEQ ID NO: 395 - DNA, 10 bp, Artificial
  Oligonucleotide
tagctagctc

SEQ ID NO: 396 - DNA, 10 bp, Artificial
  Oligonucleotide
tatgtgtgcg

SEQ ID NO: 397 - DNA, 10 bp, Artificial
  Oligonucleotide
tcgatcgtga

SEQ ID NO: 398 - DNA, 10 bp, Artificial
  Oligonucleotide
tgtactgatg

SEQ ID NO: 399 - DNA, 10 bp, Artificial
  Oligonucleotide
acagacatag

SEQ ID NO: 400 - DNA, 10 bp, Artificial
  Oligonucleotide
actcgcgctg

SEQ ID NO: 401 - DNA, 10 bp, Artificial
  Oligonucleotide
actctatgac

SEQ ID NO: 402 - DNA, 10 bp, Artificial
  Oligonucleotide
agcgcgtaca

SEQ ID NO: 403 - DNA, 10 bp, Artificial
  Oligonucleotide
agtatacagt

SEQ ID NO: 404 - DNA, 10 bp, Artificial
  Oligonucleotide
atacagtcga

SEQ ID NO: 405 - DNA, 10 bp, Artificial
  Oligonucleotide
cacgtagtat

SEQ ID NO: 406 - DNA, 10 bp, Artificial
  Oligonucleotide
cgagcacgcg

SEQ ID NO: 407 - DNA, 10 bp, Artificial
  Oligonucleotide
cgtgagagac

SEQ ID NO: 408 - DNA, 10 bp, Artificial
  Oligonucleotide
cgtgtctcta

SEQ ID NO: 409 - DNA, 10 bp, Artificial
  Oligonucleotide
tagctcactc

SEQ ID NO: 410 - DNA, 10 bp, Artificial
  Oligonucleotide
tatgtgtgcg

SEQ ID NO: 411 - DNA, 10 bp, Artificial
  Oligonucleotide
tcgatcgtga

SEQ ID NO: 412 - DNA, 10 bp, Artificial
  Oligonucleotide
tgtactgatg

SEQ ID NO: 413 - DNA, 10 bp, Artificial
  Oligonucleotide
acagacatag

SEQ ID NO: 414 - DNA, 10 bp, Artificial
  Oligonucleotide
actcgcgctg

SEQ ID NO: 415 - DNA, 10 bp, Artificial
  Oligonucleotide
actctatgac

SEQ ID NO: 416 - DNA, 10 bp, Artificial
  Oligonucleotide
agcgcgtaca

SEQ ID NO: 417 - DNA, 10 bp, Artificial
  Oligonucleotide
atacagtcga

SEQ ID NO: 418 - DNA, 10 bp, Artificial
  Oligonucleotide
cacgtagtat

SEQ ID NO: 419 - DNA, 10 bp, Artificial
  Oligonucleotide
cgagcacgcg

SEQ ID NO: 420 - DNA, 10 bp, Artificial
  Oligonucleotide
cgtgagagac

SEQ ID NO: 421 - DNA, 10 bp, Artificial
  Oligonucleotide
cgtgtctcta

SEQ ID NO: 422 - DNA, 10 bp, Artificial
  Oligonucleotide
tagatatctc

SEQ ID NO: 423 - DNA, 10 bp, Artificial
  Oligonucleotide
tatcgacagt

SEQ ID NO: 424 - DNA, 10 bp, Artificial
  Oligonucleotide
tatgtgtgcg

SEQ ID NO: 425 - DNA, 10 bp, Artificial
  Oligonucleotide
tcgatcgtga

SEQ ID NO: 426 - DNA, 10 bp, Artificial
  Oligonucleotide
tgtactgatg

SEQ ID NO: 427 - DNA, 10 bp, Artificial
  Oligonucleotide
acagacatag

SEQ ID NO: 428 - DNA, 10 bp, Artificial
  Oligonucleotide
actcgcgctg

SEQ ID NO: 429 - DNA, 10 bp, Artificial
  Oligonucleotide
actctatgac

SEQ ID NO: 430 - DNA, 10 bp, Artificial
  Oligonucleotide
agcgcgtaca

SEQ ID NO: 431 - DNA, 10 bp, Artificial
  Oligonucleotide
atacagtcga

SEQ ID NO: 432 - DNA, 10 bp, Artificial
  Oligonucleotide
cacgtagtat

SEQ ID NO: 433 - DNA, 10 bp, Artificial
  Oligonucleotide
cgagcacgcg

SEQ ID NO: 434 - DNA, 10 bp, Artificial
  Oligonucleotide
cgtgagagac

SEQ ID NO: 435 - DNA, 10 bp, Artificial
  Oligonucleotide
cgtgtctcta

SEQ ID NO: 436 - DNA, 10 bp, Artificial
  Oligonucleotide
tagctagctc

SEQ ID NO: 437 - DNA, 10 bp, Artificial
  Oligonucleotide
tatcgacagt

SEQ ID NO: 438 - DNA, 10 bp, Artificial
  Oligonucleotide
tatgtgtgcg

SEQ ID NO: 439 - DNA, 10 bp, Artificial
  Oligonucleotide
tcgatcgtga

SEQ ID NO: 440 - DNA, 10 bp, Artificial
  Oligonucleotide
tgtactgatg

SEQ ID NO: 441 - DNA, 10 bp, Artificial
  Oligonucleotide
acagacatag

SEQ ID NO: 442 - DNA, 10 bp, Artificial
  Oligonucleotide
actcgcgctg

SEQ ID NO: 443 - DNA, 10 bp, Artificial
  Oligonucleotide
actctatgac

SEQ ID NO: 444 - DNA, 10 bp, Artificial
  Oligonucleotide
agcgcgtaca

SEQ ID NO: 445 - DNA, 10 bp, Artificial
  Oligonucleotide
atacagtcga

SEQ ID NO: 446 - DNA, 10 bp, Artificial
  Oligonucleotide
cacgtagtat

SEQ ID NO: 447 - DNA, 10 bp, Artificial
  Oligonucleotide
cgagcacgcg

SEQ ID NO: 448 - DNA, 10 bp, Artificial
  Oligonucleotide
cgtgagagac

SEQ ID NO: 449 - DNA, 10 bp, Artificial
  Oligonucleotide
cgtgtctcta

SEQ ID NO: 450 - DNA, 10 bp, Artificial
  Oligonucleotide
tagatatctc

SEQ ID NO: 451 - DNA, 10 bp, Artificial
  Oligonucleotide
tagcgacagt

SEQ ID NO: 452 - DNA, 10 bp, Artificial
  Oligonucleotide
tatgtgtgcg

SEQ ID NO: 453 - DNA, 10 bp, Artificial
  Oligonucleotide
tcgatcgtga

SEQ ID NO: 454 - DNA, 10 bp, Artificial
  Oligonucleotide
tgtactgatg

SEQ ID NO: 455 - DNA, 10 bp, Artificial
  Oligonucleotide
acatatacgc

SEQ ID NO: 456 - DNA, 10 bp, Artificial
  Oligonucleotide
actcgcgctg

SEQ ID NO: 457 - DNA, 10 bp, Artificial
  Oligonucleotide
agcgcgtaca

SEQ ID NO: 458 - DNA, 10 bp, Artificial
  Oligonucleotide
agtactgtgt

SEQ ID NO: 459 - DNA, 10 bp, Artificial
  Oligonucleotide
cacagtagta

SEQ ID NO: 460 - DNA, 10 bp, Artificial
  Oligonucleotide
cgagcacgcg

SEQ ID NO: 461 - DNA, 10 bp, Artificial
  Oligonucleotide
cgtgagagac

SEQ ID NO: 462 - DNA, 10 bp, Artificial
  Oligonucleotide
cgtgtctcta

SEQ ID NO: 463 - DNA, 10 bp, Artificial
  Oligonucleotide
ctctacatcg

SEQ ID NO: 464 - DNA, 10 bp, Artificial
  Oligonucleotide
tagctagctc

SEQ ID NO: 465 - DNA, 10 bp, Artificial
  Oligonucleotide
tatcgacagt

SEQ ID NO: 466 - DNA, 10 bp, Artificial
  Oligonucleotide
tatgtgtgcg

SEQ ID NO: 467 - DNA, 10 bp, Artificial
  Oligonucleotide
tcgatcgtga

SEQ ID NO: 468 - DNA, 10 bp, Artificial
  Oligonucleotide
tgcactcgac

SEQ ID NO: 469 - DNA, 10 bp, Artificial
  Oligonucleotide
actcgcgctg

SEQ ID NO: 470 - DNA, 10 bp, Artificial
  Oligonucleotide
agcgcgtaca

SEQ ID NO: 471 - DNA, 10 bp, Artificial
  Oligonucleotide
atagtctagt

SEQ ID NO: 472 - DNA, 10 bp, Artificial
  Oligonucleotide
atcatacacg

SEQ ID NO: 473 - DNA, 10 bp, Artificial
  Oligonucleotide
cacgtagtat

SEQ ID NO: 474 - DNA, 10 bp, Artificial
  Oligonucleotide
cgagcacgcg

SEQ ID NO: 475 - DNA, 10 bp, Artificial
  Oligonucleotide
cgtgagagac

SEQ ID NO: 476 - DNA, 10 bp, Artificial
  Oligonucleotide
cgtgtctcta

SEQ ID NO: 477 - DNA, 10 bp, Artificial
  Oligonucleotide
ctactgacga

SEQ ID NO: 478 - DNA, 10 bp, Artificial
  Oligonucleotide
tagatatctc

SEQ ID NO: 479 - DNA, 10 bp, Artificial
  Oligonucleotide
tatacgcata

SEQ ID NO: 480 - DNA, 10 bp, Artificial
  Oligonucleotide
tatgtgtgcg

SEQ ID NO: 481 - DNA, 10 bp, Artificial
  Oligonucleotide
tcgatcgtga

SEQ ID NO: 482 - DNA, 10 bp, Artificial
  Oligonucleotide
tgcactcgac

SEQ ID NO: 483 - DNA, 10 bp, Artificial
  Oligonucleotide
acagacatag

SEQ ID NO: 484 - DNA, 10 bp, Artificial
  Oligonucleotide
actcgcgctg

SEQ ID NO: 485 - DNA, 10 bp, Artificial
  Oligonucleotide
agcgcgtaca

SEQ ID NO: 486 - DNA, 10 bp, Artificial
  Oligonucleotide
agtatacagt

SEQ ID NO: 487 - DNA, 10 bp, Artificial
  Oligonucleotide
atacagtcga

SEQ ID NO: 488 - DNA, 10 bp, Artificial
  Oligonucleotide
cacgtagtat

SEQ ID NO: 489 - DNA, 10 bp, Artificial
  Oligonucleotide
cgagcacgcg

SEQ ID NO: 490 - DNA, 10 bp, Artificial
  Oligonucleotide
cgtgagagac

SEQ ID NO: 491 - DNA, 10 bp, Artificial
  Oligonucleotide
cgtgtctcta

SEQ ID NO: 492 - DNA, 10 bp, Artificial
  Oligonucleotide
ctcatctacg

SEQ ID NO: 493 - DNA, 10 bp, Artificial
  Oligonucleotide
tagatatctc

SEQ ID NO: 494 - DNA, 10 bp, Artificial
  Oligonucleotide
tatgtgtgcg

SEQ ID NO: 495 - DNA, 10 bp, Artificial
  Oligonucleotide
tcgctcgagt

SEQ ID NO: 496 - DNA, 10 bp, Artificial
  Oligonucleotide
tctactgcac

SEQ ID NO: 497 - DNA, 10 bp, Artificial
  Oligonucleotide
acagacatag

SEQ ID NO: 498 - DNA, 10 bp, Artificial
  Oligonucleotide
actcgcgctg

SEQ ID NO: 499 - DNA, 10 bp, Artificial
  Oligonucleotide
agcgcgtaca

SEQ ID NO: 500 - DNA, 10 bp, Artificial
  Oligonucleotide
agtatacagt

SEQ ID NO: 501 - DNA, 10 bp, Artificial
  Oligonucleotide
atacagtcga

SEQ ID NO: 502 - DNA, 10 bp, Artificial
  Oligonucleotide
cacgtagtat

SEQ ID NO: 503 - DNA, 10 bp, Artificial
  Oligonucleotide
cgagcacgcg

SEQ ID NO: 504 - DNA, 10 bp, Artificial
  Oligonucleotide
cgtgagagac

SEQ ID NO: 505 - DNA, 10 bp, Artificial
  Oligonucleotide
cgtgtctcta

SEQ ID NO: 506 - DNA, 10 bp, Artificial
  Oligonucleotide
ctcatctacg

SEQ ID NO: 507 - DNA, 10 bp, Artificial
  Oligonucleotide
tagatatctc

SEQ ID NO: 508 - DNA, 10 bp, Artificial
  Oligonucleotide
tatgtgtgcg

SEQ ID NO: 509 - DNA, 10 bp, Artificial
  Oligonucleotide
tcgctcgagt

SEQ ID NO: 510 - DNA, 10 bp, Artificial
  Oligonucleotide
tgtactgatg

SEQ ID NO: 511 - DNA, 10 bp, Artificial
  Oligonucleotide
acagacatag

SEQ ID NO: 512 - DNA, 10 bp, Artificial
  Oligonucleotide
actcgcgctg

SEQ ID NO: 513 - DNA, 10 bp, Artificial
  Oligonucleotide
actctatgac

SEQ ID NO: 514 - DNA, 10 bp, Artificial
  Oligonucleotide
agcgcgtaca

SEQ ID NO: 515 - DNA, 10 bp, Artificial
  Oligonucleotide
agtatacagt

SEQ ID NO: 516 - DNA, 10 bp, Artificial
  Oligonucleotide
atacagtcga

SEQ ID NO: 517 - DNA, 10 bp, Artificial
  Oligonucleotide
cacgtagtat

SEQ ID NO: 518 - DNA, 10 bp, Artificial
  Oligonucleotide
cgagcacgcg

SEQ ID NO: 519 - DNA, 10 bp, Artificial
  Oligonucleotide
cgtgagagac

SEQ ID NO: 520 - DNA, 10 bp, Artificial
  Oligonucleotide
cgtgtctcta

SEQ ID NO: 521 - DNA, 10 bp, Artificial
  Oligonucleotide
tagatatctc

SEQ ID NO: 522 - DNA, 10 bp, Artificial
  Oligonucleotide
tatgtgtgcg

SEQ ID NO: 523 - DNA, 10 bp, Artificial
  Oligonucleotide
tcgctcgagt

SEQ ID NO: 524 - DNA, 10 bp, Artificial
  Oligonucleotide
tgtactgatg

SEQ ID NO: 525 - DNA, 10 bp, Artificial
  Oligonucleotide
acacgactgc

SEQ ID NO: 526 - DNA, 10 bp, Artificial
  Oligonucleotide
agtatacagt

SEQ ID NO: 527 - DNA, 10 bp, Artificial
  Oligonucleotide
atactcatcg

SEQ ID NO: 528 - DNA, 10 bp, Artificial
  Oligonucleotide
atcgatacta

SEQ ID NO: 529 - DNA, 10 bp, Artificial
  Oligonucleotide
cgagcacgcg

SEQ ID NO: 530 - DNA, 10 bp, Artificial
  Oligonucleotide
cgtgagagac

SEQ ID NO: 531 - DNA, 10 bp, Artificial
  Oligonucleotide
cgtgtctcta

SEQ ID NO: 532 - DNA, 10 bp, Artificial
  Oligonucleotide
ctacagtgta

SEQ ID NO: 533 - DNA, 10 bp, Artificial
  Oligonucleotide
ctctacgcac

SEQ ID NO: 534 - DNA, 10 bp, Artificial
  Oligonucleotide
tagctctgac

SEQ ID NO: 535 - DNA, 10 bp, Artificial
  Oligonucleotide
tcacatagag

SEQ ID NO: 536 - DNA, 10 bp, Artificial
  Oligonucleotide
tctgtgtgcg

SEQ ID NO: 537 - DNA, 10 bp, Artificial
  Oligonucleotide
tgatagtcgc

SEQ ID NO: 538 - DNA, 10 bp, Artificial
  Oligonucleotide
tgtcgcgatg

SEQ ID NO: 539 - DNA, 10 bp, Artificial
  Oligonucleotide
acactctgtc

SEQ ID NO: 540 - DNA, 10 bp, Artificial
  Oligonucleotide
agtatacagt

SEQ ID NO: 541 - DNA, 10 bp, Artificial
  Oligonucleotide
atagcgtcag

SEQ ID NO: 542 - DNA, 10 bp, Artificial
  Oligonucleotide
atcgatacta

SEQ ID NO: 543 - DNA, 10 bp, Artificial
  Oligonucleotide
cgagcacgcg

SEQ ID NO: 544 - DNA, 10 bp, Artificial
  Oligonucleotide
cgtgagagac

SEQ ID NO: 545 - DNA, 10 bp, Artificial
  Oligonucleotide
cgtgtctcta

SEQ ID NO: 546 - DNA, 10 bp, Artificial
  Oligonucleotide
ctacgagtgt

SEQ ID NO: 547 - DNA, 10 bp, Artificial
  Oligonucleotide
ctctacgcac

SEQ ID NO: 548 - DNA, 10 bp, Artificial
  Oligonucleotide
tactgacatc

SEQ ID NO: 549 - DNA, 10 bp, Artificial
  Oligonucleotide
tcacatagag

SEQ ID NO: 550 - DNA, 10 bp, Artificial
  Oligonucleotide
tctgtgtgcg

SEQ ID NO: 551 - DNA, 10 bp, Artificial
  Oligonucleotide
tgatagtcgc

SEQ ID NO: 552 - DNA, 10 bp, Artificial
  Oligonucleotide
tgtcgcgatg

SEQ ID NO: 553 - DNA, 10 bp, Artificial
  Oligonucleotide
acatactcgt

SEQ ID NO: 554 - DNA, 10 bp, Artificial
  Oligonucleotide
agctcgtaga

SEQ ID NO: 555 - DNA, 10 bp, Artificial
  Oligonucleotide
agtatgtgcg

SEQ ID NO: 556 - DNA, 10 bp, Artificial
  Oligonucleotide
atctgacgtc

SEQ ID NO: 557 - DNA, 10 bp, Artificial
  Oligonucleotide
cgagcacgcg

SEQ ID NO: 558 - DNA, 10 bp, Artificial
  Oligonucleotide
cgtgagagac

SEQ ID NO: 559 - DNA, 10 bp, Artificial
  Oligonucleotide
cgtgtctcta

SEQ ID NO: 560 - DNA, 10 bp, Artificial
  Oligonucleotide
ctacgagtgt

SEQ ID NO: 561 - DNA, 10 bp, Artificial
  Oligonucleotide
ctctacacag

SEQ ID NO: 562 - DNA, 10 bp, Artificial
  Oligonucleotide
tacatagata

SEQ ID NO: 563 - DNA, 10 bp, Artificial
  Oligonucleotide
tagcgtgcac

SEQ ID NO: 564 - DNA, 10 bp, Artificial
  Oligonucleotide
tcactgtctg

SEQ ID NO: 565 - DNA, 10 bp, Artificial
  Oligonucleotide
tcgtcatact

SEQ ID NO: 566 - DNA, 10 bp, Artificial
  Oligonucleotide
tgtcgcgatg

SEQ ID NO: 567 - DNA, 10 bp, Artificial
  Oligonucleotide
acatactcgc

SEQ ID NO: 568 - DNA, 10 bp, Artificial
  Oligonucleotide
agctcgtaga

SEQ ID NO: 569 - DNA, 10 bp, Artificial
  Oligonucleotide
agtatgtgcg

SEQ ID NO: 570 - DNA, 10 bp, Artificial
  Oligonucleotide
atctgacgtc

SEQ ID NO: 571 - DNA, 10 bp, Artificial
  Oligonucleotide
cgagcacgcg

SEQ ID NO: 572 - DNA, 10 bp, Artificial
  Oligonucleotide
cgtgagagac

SEQ ID NO: 573 - DNA, 10 bp, Artificial
  Oligonucleotide
cgtgtctcta

SEQ ID NO: 574 - DNA, 10 bp, Artificial
  Oligonucleotide
ctacgagtgt

SEQ ID NO: 575 - DNA, 10 bp, Artificial
  Oligonucleotide
ctctacacag

SEQ ID NO: 576 - DNA, 10 bp, Artificial
  Oligonucleotide
tacatagata

SEQ ID NO: 577 - DNA, 10 bp, Artificial
  Oligonucleotide
tagcgtgcac

SEQ ID NO: 578 - DNA, 10 bp, Artificial
  Oligonucleotide
tcactgtctg

SEQ ID NO: 579 - DNA, 10 bp, Artificial
  Oligonucleotide
tcgtcatact

SEQ ID NO: 580 - DNA, 10 bp, Artificial
  Oligonucleotide
tgtcgcgatg

SEQ ID NO: 581 - DNA, 10 bp, Artificial
  Oligonucleotide
acacacactc

SEQ ID NO: 582 - DNA, 10 bp, Artificial
  Oligonucleotide
acatctgtag

SEQ ID NO: 583 - DNA, 10 bp, Artificial
  Oligonucleotide
acgcgcgtgt

SEQ ID NO: 584 - DNA, 10 bp, Artificial
  Oligonucleotide
agcgatatac

SEQ ID NO: 585 - DNA, 10 bp, Artificial
  Oligonucleotide
agtagtgaga

SEQ ID NO: 586 - DNA, 10 bp, Artificial
  Oligonucleotide
cgagagcgcg

SEQ ID NO: 587 - DNA, 10 bp, Artificial
  Oligonucleotide
cgtcatacgt

SEQ ID NO: 588 - DNA, 10 bp, Artificial
  Oligonucleotide
cgtgtctcta

SEQ ID NO: 589 - DNA, 10 bp, Artificial
  Oligonucleotide
ctactagtct

SEQ ID NO: 590 - DNA, 10 bp, Artificial
  Oligonucleotide
ctcgcgacag

SEQ ID NO: 591 - DNA, 10 bp, Artificial
  Oligonucleotide
tagactcgat

SEQ ID NO: 592 - DNA, 10 bp, Artificial
  Oligonucleotide
tatgagagtc

SEQ ID NO: 593 - DNA, 10 bp, Artificial
  Oligonucleotide
tgctgtagcg

SEQ ID NO: 594 - DNA, 10 bp, Artificial
  Oligonucleotide
tgtacgctgc

SEQ ID NO: 595 - DNA, 10 bp, Artificial
  Oligonucleotide
acatctgtag

SEQ ID NO: 596 - DNA, 10 bp, Artificial
  Oligonucleotide
acgcgcgtgt

SEQ ID NO: 597 - DNA, 10 bp, Artificial
  Oligonucleotide
agcgatatac

SEQ ID NO: 598 - DNA, 10 bp, Artificial
  Oligonucleotide
agtagtgaga

SEQ ID NO: 599 - DNA, 10 bp, Artificial
  Oligonucleotide
cgagagcgcg

SEQ ID NO: 600 - DNA, 10 bp, Artificial
  Oligonucleotide
cgtcatacgt

SEQ ID NO: 601 - DNA, 10 bp, Artificial
  Oligonucleotide
cgtgtctcta

SEQ ID NO: 602 - DNA, 10 bp, Artificial
  Oligonucleotide
ctactagtct

SEQ ID NO: 603 - DNA, 10 bp, Artificial
  Oligonucleotide
ctcgcgacag

SEQ ID NO: 604 - DNA, 10 bp, Artificial
  Oligonucleotide
tagactcgat

SEQ ID NO: 605 - DNA, 10 bp, Artificial
  Oligonucleotide
tatatacaca

SEQ ID NO: 606 - DNA, 10 bp, Artificial
  Oligonucleotide
tcacagagta

SEQ ID NO: 607 - DNA, 10 bp, Artificial
  Oligonucleotide
tgctgtagcg

SEQ ID NO: 608 - DNA, 10 bp, Artificial
  Oligonucleotide
tgtacgctgc

SEQ ID NO: 609 - DNA, 10 bp, Artificial
  Oligonucleotide
acgcgcgtgt

SEQ ID NO: 610 - DNA, 10 bp, Artificial
  Oligonucleotide
agagacatac

SEQ ID NO: 611 - DNA, 10 bp, Artificial
  Oligonucleotide
atagtagtct

SEQ ID NO: 612 - DNA, 10 bp, Artificial
  Oligonucleotide
atctgacgag

SEQ ID NO: 613 - DNA, 10 bp, Artificial
  Oligonucleotide
cacgctatcg

SEQ ID NO: 614 - DNA, 10 bp, Artificial
  Oligonucleotide
cgagagcgcg

SEQ ID NO: 615 - DNA, 10 bp, Artificial
  Oligonucleotide
cgtcatacgt

SEQ ID NO: 616 - DNA, 10 bp, Artificial
  Oligonucleotide
cgtgtctcta

SEQ ID NO: 617 - DNA, 10 bp, Artificial
  Oligonucleotide
ctcacgtcac

SEQ ID NO: 618 - DNA, 10 bp, Artificial
  Oligonucleotide
tagctctgac

SEQ ID NO: 619 - DNA, 10 bp, Artificial
  Oligonucleotide
tatacatgtg

SEQ ID NO: 620 - DNA, 10 bp, Artificial
  Oligonucleotide
tcactgagta

SEQ ID NO: 621 - DNA, 10 bp, Artificial
  Oligonucleotide
tctatacaca

SEQ ID NO: 622 - DNA, 10 bp, Artificial
  Oligonucleotide
tgcgtagatg

SEQ ID NO: 623 - DNA, 10 bp, Artificial
  Oligonucleotide
acacatatag

SEQ ID NO: 624 - DNA, 10 bp, Artificial
  Oligonucleotide
acgcgcgtgt

SEQ ID NO: 625 - DNA, 10 bp, Artificial
  Oligonucleotide
agacgcactc

SEQ ID NO: 626 - DNA, 10 bp, Artificial
  Oligonucleotide
atatacgtca

SEQ ID NO: 627 - DNA, 10 bp, Artificial
  Oligonucleotide
atgtctagcg

SEQ ID NO: 628 - DNA, 10 bp, Artificial
  Oligonucleotide
cgagagcgcg

SEQ ID NO: 629 - DNA, 10 bp, Artificial
  Oligonucleotide
cgctcgacag

SEQ ID NO: 630 - DNA, 10 bp, Artificial
  Oligonucleotide
cgtgtctcta

SEQ ID NO: 631 - DNA, 10 bp, Artificial
  Oligonucleotide
ctcgtagatc

SEQ ID NO: 632 - DNA, 10 bp, Artificial
  Oligonucleotide
tactgagcta

SEQ ID NO: 633 - DNA, 10 bp, Artificial
  Oligonucleotide
tagcgatgac

SEQ ID NO: 634 - DNA, 10 bp, Artificial
  Oligonucleotide
tctatgtgtg

SEQ ID NO: 635 - DNA, 10 bp, Artificial
  Oligonucleotide
tctcactcgc

SEQ ID NO: 636 - DNA, 10 bp, Artificial
  Oligonucleotide
tgagtacagt

SEQ ID NO: 637 - DNA, 10 bp, Artificial
  Oligonucleotide
acacatatag

SEQ ID NO: 638 - DNA, 10 bp, Artificial
  Oligonucleotide
acgcgcgtgt

SEQ ID NO: 639 - DNA, 10 bp, Artificial
  Oligonucleotide
agacgcactc

SEQ ID NO: 640 - DNA, 10 bp, Artificial
  Oligonucleotide
atatacgtca

SEQ ID NO: 641 - DNA, 10 bp, Artificial
  Oligonucleotide
atgtctagcg

SEQ ID NO: 642 - DNA, 10 bp, Artificial
  Oligonucleotide
cgagagcgcg

SEQ ID NO: 643 - DNA, 10 bp, Artificial
  Oligonucleotide
cgctcgacag

SEQ ID NO: 644 - DNA, 10 bp, Artificial
  Oligonucleotide
cgtgtctcta

SEQ ID NO: 645 - DNA, 10 bp, Artificial
  Oligonucleotide
ctcgtagatc

SEQ ID NO: 646 - DNA, 10 bp, Artificial
  Oligonucleotide
tactgagcta

SEQ ID NO: 647 - DNA, 10 bp, Artificial
  Oligonucleotide
tagcgatgac

SEQ ID NO: 648 - DNA, 10 bp, Artificial
  Oligonucleotide
tctatgtgtg

SEQ ID NO: 649 - DNA, 10 bp, Artificial
  Oligonucleotide
tctcactcgc

SEQ ID NO: 650 - DNA, 10 bp, Artificial
  Oligonucleotide
tgagtacaga

SEQ ID NO: 651 - DNA, 10 bp, Artificial
  Oligonucleotide
acatatacag

SEQ ID NO: 652 - DNA, 10 bp, Artificial
  Oligonucleotide
acgcgcgtgt

SEQ ID NO: 653 - DNA, 10 bp, Artificial
  Oligonucleotide
agacgcactc

SEQ ID NO: 654 - DNA, 10 bp, Artificial
  Oligonucleotide
atgactagcg

SEQ ID NO: 655 - DNA, 10 bp, Artificial
  Oligonucleotide
cacagtagta

SEQ ID NO: 656 - DNA, 10 bp, Artificial
  Oligonucleotide
cgagagcgcg

SEQ ID NO: 657 - DNA, 10 bp, Artificial
  Oligonucleotide
cgctactgac

SEQ ID NO: 658 - DNA, 10 bp, Artificial
  Oligonucleotide
cgtgtctcta

SEQ ID NO: 659 - DNA, 10 bp, Artificial
  Oligonucleotide
ctcgcgacag

SEQ ID NO: 660 - DNA, 10 bp, Artificial
  Oligonucleotide
tacatcgagc

SEQ ID NO: 661 - DNA, 10 bp, Artificial
  Oligonucleotide
tagtactcgt

SEQ ID NO: 662 - DNA, 10 bp, Artificial
  Oligonucleotide
tctacactca

SEQ ID NO: 663 - DNA, 10 bp, Artificial
  Oligonucleotide
tctatgtgtg

SEQ ID NO: 664 - DNA, 10 bp, Artificial
  Oligonucleotide
tgtcagatac

SEQ ID NO: 665 - DNA, 10 bp, Artificial
  Oligonucleotide
acacacactc

SEQ ID NO: 666 - DNA, 10 bp, Artificial
  Oligonucleotide
acatctgtag

SEQ ID NO: 667 - DNA, 10 bp, Artificial
  Oligonucleotide
acgcgcgtgt

SEQ ID NO: 668 - DNA, 10 bp, Artificial
  Oligonucleotide
agcgatatac

SEQ ID NO: 669 - DNA, 10 bp, Artificial
  Oligonucleotide
agtagtgaga

SEQ ID NO: 670 - DNA, 10 bp, Artificial
  Oligonucleotide
cgagagcgcg

SEQ ID NO: 671 - DNA, 10 bp, Artificial
  Oligonucleotide
cgtgtctcta

SEQ ID NO: 672 - DNA, 10 bp, Artificial
  Oligonucleotide
ctactagtct

SEQ ID NO: 673 - DNA, 10 bp, Artificial
  Oligonucleotide
ctcgcgacag

SEQ ID NO: 674 - DNA, 10 bp, Artificial
  Oligonucleotide
tagactcgat

SEQ ID NO: 675 - DNA, 10 bp, Artificial
  Oligonucleotide
tatcatcacg

SEQ ID NO: 676 - DNA, 10 bp, Artificial
  Oligonucleotide
tatgagagtc

SEQ ID NO: 677 - DNA, 10 bp, Artificial
  Oligonucleotide
tgctgtagcg

SEQ ID NO: 678 - DNA, 10 bp, Artificial
  Oligonucleotide
tgtacgctgc

SEQ ID NO: 679 - DNA, 10 bp, Artificial
  Oligonucleotide
acactctcag

SEQ ID NO: 680 - DNA, 10 bp, Artificial
  Oligonucleotide
acgcgcgtgt

SEQ ID NO: 681 - DNA, 10 bp, Artificial
  Oligonucleotide
acgtagacat

SEQ ID NO: 682 - DNA, 10 bp, Artificial
  Oligonucleotide
agacagtata

SEQ ID NO: 683 - DNA, 10 bp, Artificial
  Oligonucleotide
agtatgtgcg

SEQ ID NO: 684 - DNA, 10 bp, Artificial
  Oligonucleotide
cgagcgcacg

SEQ ID NO: 685 - DNA, 10 bp, Artificial
  Oligonucleotide
cgtgtctcta

SEQ ID NO: 686 - DNA, 10 bp, Artificial
  Oligonucleotide
ctacgatctc

SEQ ID NO: 687 - DNA, 10 bp, Artificial
  Oligonucleotide
ctctacgaga

SEQ ID NO: 688 - DNA, 10 bp, Artificial
  Oligonucleotide
tagtcacgca

SEQ ID NO: 689 - DNA, 10 bp, Artificial
  Oligonucleotide
tatagcagtg

SEQ ID NO: 690 - DNA, 10 bp, Artificial
  Oligonucleotide
tcgtctgatc

SEQ ID NO: 691 - DNA, 10 bp, Artificial
  Oligonucleotide
tctgatatag

SEQ ID NO: 692 - DNA, 10 bp, Artificial
  Oligonucleotide
tgcactacgt

SEQ ID NO: 693 - DNA, 10 bp, Artificial
  Oligonucleotide
acagtcgcac

SEQ ID NO: 694 - DNA, 10 bp, Artificial
  Oligonucleotide
acgcgcgtgt

SEQ ID NO: 695 - DNA, 10 bp, Artificial
  Oligonucleotide
agactgtgag

SEQ ID NO: 696 - DNA, 10 bp, Artificial
  Oligonucleotide
agtatacatg

SEQ ID NO: 697 - DNA, 10 bp, Artificial
  Oligonucleotide
cactactagc

SEQ ID NO: 698 - DNA, 10 bp, Artificial
  Oligonucleotide
cgagcacgcg

SEQ ID NO: 699 - DNA, 10 bp, Artificial
  Oligonucleotide
cgtgtctcta

SEQ ID NO: 700 - DNA, 10 bp, Artificial
  Oligonucleotide
ctacgcgata

SEQ ID NO: 701 - DNA, 10 bp, Artificial
  Oligonucleotide
ctcagtgtcg

SEQ ID NO: 702 - DNA, 10 bp, Artificial
  Oligonucleotide
tatcgctcag

SEQ ID NO: 703 - DNA, 10 bp, Artificial
  Oligonucleotide
tatgtagtga

SEQ ID NO: 704 - DNA, 10 bp, Artificial
  Oligonucleotide
tcgcatacga

SEQ ID NO: 705 - DNA, 10 bp, Artificial
  Oligonucleotide
tgagatatac

SEQ ID NO: 706 - DNA, 10 bp, Artificial
  Oligonucleotide
tgtcgagact

SEQ ID NO: 707 - DNA, 10 bp, Artificial
  Oligonucleotide
acatactatg

SEQ ID NO: 708 - DNA, 10 bp, Artificial
  Oligonucleotide
acgcgcgtgt

SEQ ID NO: 709 - DNA, 10 bp, Artificial
  Oligonucleotide
agtagatagt

SEQ ID NO: 710 - DNA, 10 bp, Artificial
  Oligonucleotide
atgtgacgac

SEQ ID NO: 711 - DNA, 10 bp, Artificial
  Oligonucleotide
cagatcgtac

SEQ ID NO: 712 - DNA, 10 bp, Artificial
  Oligonucleotide
cgagcacgcg

SEQ ID NO: 713 - DNA, 10 bp, Artificial
  Oligonucleotide
cgtgtctcta

SEQ ID NO: 714 - DNA, 10 bp, Artificial
  Oligonucleotide
ctacgcgata

SEQ ID NO: 715 - DNA, 10 bp, Artificial
  Oligonucleotide
ctcagtgtcg

SEQ ID NO: 716 - DNA, 10 bp, Artificial
  Oligonucleotide
tagacactga

SEQ ID NO: 717 - DNA, 10 bp, Artificial
  Oligonucleotide
tatctagcgt

SEQ ID NO: 718 - DNA, 10 bp, Artificial
  Oligonucleotide
tcgtatgaga

SEQ ID NO: 719 - DNA, 10 bp, Artificial
  Oligonucleotide
tctcgctcac

SEQ ID NO: 720 - DNA, 10 bp, Artificial
  Oligonucleotide
tgctatactc

SEQ ID NO: 721 - DNA, 10 bp, Artificial
  Oligonucleotide
acagtcgcac

SEQ ID NO: 722 - DNA, 10 bp, Artificial
  Oligonucleotide
acgcgcgtgt

SEQ ID NO: 723 - DNA, 10 bp, Artificial
  Oligonucleotide
agtatagaca

SEQ ID NO: 724 - DNA, 10 bp, Artificial
  Oligonucleotide
atgactacag

SEQ ID NO: 725 - DNA, 10 bp, Artificial
  Oligonucleotide
cacgtagatg

SEQ ID NO: 726 - DNA, 10 bp, Artificial
  Oligonucleotide
cgagagcgag

SEQ ID NO: 727 - DNA, 10 bp, Artificial
  Oligonucleotide
cgtcatacgt

SEQ ID NO: 728 - DNA, 10 bp, Artificial
  Oligonucleotide
cgtgtctcta

SEQ ID NO: 729 - DNA, 10 bp, Artificial
  Oligonucleotide
ctcacgctcg

SEQ ID NO: 730 - DNA, 10 bp, Artificial
  Oligonucleotide
tacgatcact

SEQ ID NO: 731 - DNA, 10 bp, Artificial
  Oligonucleotide
tagcatgtag

SEQ ID NO: 732 - DNA, 10 bp, Artificial
  Oligonucleotide
tatacagcgc

SEQ ID NO: 733 - DNA, 10 bp, Artificial
  Oligonucleotide
tcgagatatc

SEQ ID NO: 734 - DNA, 10 bp, Artificial
  Oligonucleotide
tctgtgtgcg

SEQ ID NO: 735 - DNA, 10 bp, Artificial
  Oligonucleotide
acagtcgcac

SEQ ID NO: 736 - DNA, 10 bp, Artificial
  Oligonucleotide
acgcgcgtgt

SEQ ID NO: 737 - DNA, 10 bp, Artificial
  Oligonucleotide
agactgagtc

SEQ ID NO: 738 - DNA, 10 bp, Artificial
  Oligonucleotide
atctacagag

SEQ ID NO: 739 - DNA, 10 bp, Artificial
  Oligonucleotide
cacgtagatg

SEQ ID NO: 740 - DNA, 10 bp, Artificial
  Oligonucleotide
cgagagcgag

SEQ ID NO: 741 - DNA, 10 bp, Artificial
  Oligonucleotide
cgtcatacgt

SEQ ID NO: 742 - DNA, 10 bp, Artificial
  Oligonucleotide
cgtgtctcta

SEQ ID NO: 743 - DNA, 10 bp, Artificial
  Oligonucleotide
ctcacgctcg

SEQ ID NO: 744 - DNA, 10 bp, Artificial
  Oligonucleotide
tacgatcact

SEQ ID NO: 745 - DNA, 10 bp, Artificial
  Oligonucleotide
tagcatgtag

SEQ ID NO: 746 - DNA, 10 bp, Artificial
  Oligonucleotide
tatacgcata

SEQ ID NO: 747 - DNA, 10 bp, Artificial
  Oligonucleotide
tcgagatatc

SEQ ID NO: 748 - DNA, 10 bp, Artificial
  Oligonucleotide
tctgtgtgcg

SEQ ID NO: 749 - DNA, 10 bp, Artificial
  Oligonucleotide
acactatctg

SEQ ID NO: 750 - DNA, 10 bp, Artificial
  Oligonucleotide
acgcgcgtgt

SEQ ID NO: 751 - DNA, 10 bp, Artificial
  Oligonucleotide
agcgatatac

SEQ ID NO: 752 - DNA, 10 bp, Artificial
  Oligonucleotide
agtctcgacg

SEQ ID NO: 753 - DNA, 10 bp, Artificial
  Oligonucleotide
atacagagtc

SEQ ID NO: 754 - DNA, 10 bp, Artificial
  Oligonucleotide
cacgtgacgc

SEQ ID NO: 755 - DNA, 10 bp, Artificial
  Oligonucleotide
cgagagcgag

SEQ ID NO: 756 - DNA, 10 bp, Artificial
  Oligonucleotide
cgtgtctcta

SEQ ID NO: 757 - DNA, 10 bp, Artificial
  Oligonucleotide
ctctagtaga

SEQ ID NO: 758 - DNA, 10 bp, Artificial
  Oligonucleotide
tagtcagtat

SEQ ID NO: 759 - DNA, 10 bp, Artificial
  Oligonucleotide
tatatacaca

SEQ ID NO: 760 - DNA, 10 bp, Artificial
  Oligonucleotide
tctcgctcac

SEQ ID NO: 761 - DNA, 10 bp, Artificial
  Oligonucleotide
tctgtgtgcg

SEQ ID NO: 762 - DNA, 10 bp, Artificial
  Oligonucleotide
tgagctacgt

SEQ ID NO: 763 - DNA, 10 bp, Artificial
  Oligonucleotide
acgcgcgtga

SEQ ID NO: 764 - DNA, 10 bp, Artificial
  Oligonucleotide
actgtagagc

SEQ ID NO: 765 - DNA, 10 bp, Artificial
  Oligonucleotide
agagacatac

SEQ ID NO: 766 - DNA, 10 bp, Artificial
  Oligonucleotide
atacgtgctc

SEQ ID NO: 767 - DNA, 10 bp, Artificial
  Oligonucleotide
cactacactg

SEQ ID NO: 768 - DNA, 10 bp, Artificial
  Oligonucleotide
cgagagcgcg

SEQ ID NO: 769 - DNA, 10 bp, Artificial
  Oligonucleotide
cgtcatacgt

SEQ ID NO: 770 - DNA, 10 bp, Artificial
  Oligonucleotide
cgtgtctcta

SEQ ID NO: 771 - DNA, 10 bp, Artificial
  Oligonucleotide
ctcagtgtag

SEQ ID NO: 772 - DNA, 10 bp, Artificial
  Oligonucleotide
tagatagtgt

SEQ ID NO: 773 - DNA, 10 bp, Artificial
  Oligonucleotide
tatcgacgca

SEQ ID NO: 774 - DNA, 10 bp, Artificial
  Oligonucleotide
tctacgatat

SEQ ID NO: 775 - DNA, 10 bp, Artificial
  Oligonucleotide
tctatctgcg

SEQ ID NO: 776 - DNA, 10 bp, Artificial
  Oligonucleotide
tgcactcgac

SEQ ID NO: 777 - DNA, 10 bp, Artificial
  Oligonucleotide
acgcgcgtga

SEQ ID NO: 778 - DNA, 10 bp, Artificial
  Oligonucleotide
agacgcactc

SEQ ID NO: 779 - DNA, 10 bp, Artificial
  Oligonucleotide
agtagagtat

SEQ ID NO: 780 - DNA, 10 bp, Artificial
  Oligonucleotide
atcgtgacag

SEQ ID NO: 781 - DNA, 10 bp, Artificial
  Oligonucleotide
atgactagcg

SEQ ID NO: 782 - DNA, 10 bp, Artificial
  Oligonucleotide
cacactgtag

SEQ ID NO: 783 - DNA, 10 bp, Artificial
  Oligonucleotide
cgagagcgcg

SEQ ID NO: 784 - DNA, 10 bp, Artificial
  Oligonucleotide
cgtcatacgt

SEQ ID NO: 785 - DNA, 10 bp, Artificial
  Oligonucleotide
cgtgtctcta

SEQ ID NO: 786 - DNA, 10 bp, Artificial
  Oligonucleotide
tagtcacgca

SEQ ID NO: 787 - DNA, 10 bp, Artificial
  Oligonucleotide
tatctagatc

SEQ ID NO: 788 - DNA, 10 bp, Artificial
  Oligonucleotide
tcacgataca

SEQ ID NO: 789 - DNA, 10 bp, Artificial
  Oligonucleotide
tctatgtgtg

SEQ ID NO: 790 - DNA, 10 bp, Artificial
  Oligonucleotide
tgcagtcgac

SEQ ID NO: 791 - DNA, 10 bp, Artificial
  Oligonucleotide
acgcgcgtga

SEQ ID NO: 792 - DNA, 10 bp, Artificial
  Oligonucleotide
actgagtact

SEQ ID NO: 793 - DNA, 10 bp, Artificial
  Oligonucleotide
agatcgtatc

SEQ ID NO: 794 - DNA, 10 bp, Artificial
  Oligonucleotide
atactctcag

SEQ ID NO: 795 - DNA, 10 bp, Artificial
  Oligonucleotide
atgtacagcg

SEQ ID NO: 796 - DNA, 10 bp, Artificial
  Oligonucleotide
cgagagcgcg

SEQ ID NO: 797 - DNA, 10 bp, Artificial
  Oligonucleotide
cgcagtatag

SEQ ID NO: 798 - DNA, 10 bp, Artificial
  Oligonucleotide
cgtgtctcta

SEQ ID NO: 799 - DNA, 10 bp, Artificial
  Oligonucleotide
ctacgacata

SEQ ID NO: 800 - DNA, 10 bp, Artificial
  Oligonucleotide
tagagactgt

SEQ ID NO: 801 - DNA, 10 bp, Artificial
  Oligonucleotide
tagcgtgcac

SEQ ID NO: 802 - DNA, 10 bp, Artificial
  Oligonucleotide
tcgtctagat

SEQ ID NO: 803 - DNA, 10 bp, Artificial
  Oligonucleotide
tctatgtgtg

SEQ ID NO: 804 - DNA, 10 bp, Artificial
  Oligonucleotide
tctcactcgc

SEQ ID NO: 805 - DNA, 10 bp, Artificial
  Oligonucleotide
acagtcgcac

SEQ ID NO: 806 - DNA, 10 bp, Artificial
  Oligonucleotide
actgacagtg

SEQ ID NO: 807 - DNA, 10 bp, Artificial
  Oligonucleotide
agatagacta

SEQ ID NO: 808 - DNA, 10 bp, Artificial
  Oligonucleotide
agcgctacag

SEQ ID NO: 809 - DNA, 10 bp, Artificial
  Oligonucleotide
atctatctcg

SEQ ID NO: 810 - DNA, 10 bp, Artificial
  Oligonucleotide
cgagagcgcg

SEQ ID NO: 811 - DNA, 10 bp, Artificial
  Oligonucleotide
cgtcatacgt

SEQ ID NO: 812 - DNA, 10 bp, Artificial
  Oligonucleotide
cgtgtctcta

SEQ ID NO: 813 - DNA, 10 bp, Artificial
  Oligonucleotide
ctacgtgaca

SEQ ID NO: 814 - DNA, 10 bp, Artificial
  Oligonucleotide
tagtactgct

SEQ ID NO: 815 - DNA, 10 bp, Artificial
  Oligonucleotide
tatctcagac

SEQ ID NO: 816 - DNA, 10 bp, Artificial
  Oligonucleotide
tcagagtaga

SEQ ID NO: 817 - DNA, 10 bp, Artificial
  Oligonucleotide
tcgctgtgtg

SEQ ID NO: 818 - DNA, 10 bp, Artificial
  Oligonucleotide
tgcgacgatc

SEQ ID NO: 819 - DNA, 10 bp, Artificial
  Oligonucleotide
acacactgtg

SEQ ID NO: 820 - DNA, 10 bp, Artificial
  Oligonucleotide
acgctagata

SEQ ID NO: 821 - DNA, 10 bp, Artificial
  Oligonucleotide
actgtgcgtc

SEQ ID NO: 822 - DNA, 10 bp, Artificial
  Oligonucleotide
atacgcacga

SEQ ID NO: 823 - DNA, 10 bp, Artificial
  Oligonucleotide
cgagagcgcg

SEQ ID NO: 824 - DNA, 10 bp, Artificial
  Oligonucleotide
cgcacgtcac

SEQ ID NO: 825 - DNA, 10 bp, Artificial
  Oligonucleotide
cgtcatacgt

SEQ ID NO: 826 - DNA, 10 bp, Artificial
  Oligonucleotide
cgtgtctcta

SEQ ID NO: 827 - DNA, 10 bp, Artificial
  Oligonucleotide
ctactagtct

SEQ ID NO: 828 - DNA, 10 bp, Artificial
  Oligonucleotide
tacatcgagc

SEQ ID NO: 829 - DNA, 10 bp, Artificial
  Oligonucleotide
tagtgtatac

SEQ ID NO: 830 - DNA, 10 bp, Artificial
  Oligonucleotide
tctatgacag

SEQ ID NO: 831 - DNA, 10 bp, Artificial
  Oligonucleotide
tgatacactc

SEQ ID NO: 832 - DNA, 10 bp, Artificial
  Oligonucleotide
tgtcgagtga

SEQ ID NO: 833 - DNA, 10 bp, Artificial
  Oligonucleotide
acgatctgag

SEQ ID NO: 834 - DNA, 10 bp, Artificial
  Oligonucleotide
actcgcgctg

SEQ ID NO: 835 - DNA, 10 bp, Artificial
  Oligonucleotide
agtatagaca

SEQ ID NO: 836 - DNA, 10 bp, Artificial
  Oligonucleotide
atactcatcg

SEQ ID NO: 837 - DNA, 10 bp, Artificial
  Oligonucleotide
atgtgacgtc

SEQ ID NO: 838 - DNA, 10 bp, Artificial
  Oligonucleotide
cacgtagtat

SEQ ID NO: 839 - DNA, 10 bp, Artificial
  Oligonucleotide
cgagagcgcg

SEQ ID NO: 840 - DNA, 10 bp, Artificial
  Oligonucleotide
cgcacgtcac

SEQ ID NO: 841 - DNA, 10 bp, Artificial
  Oligonucleotide
cgtcatacgt

SEQ ID NO: 842 - DNA, 10 bp, Artificial
  Oligonucleotide
cgtgtctcta

SEQ ID NO: 843 - DNA, 10 bp, Artificial
  Oligonucleotide
tagatatctc

SEQ ID NO: 844 - DNA, 10 bp, Artificial
  Oligonucleotide
tagcacgagt

SEQ ID NO: 845 - DNA, 10 bp, Artificial
  Oligonucleotide
tatagtgtga

SEQ ID NO: 846 - DNA, 10 bp, Artificial
  Oligonucleotide
tctctgatac

SEQ ID NO: 847 - DNA, 10 bp, Artificial
  Oligonucleotide
acatctcgtg

SEQ ID NO: 848 - DNA, 10 bp, Artificial
  Oligonucleotide
acgctgacag

SEQ ID NO: 849 - DNA, 10 bp, Artificial
  Oligonucleotide
agacgcactc

SEQ ID NO: 850 - DNA, 10 bp, Artificial
  Oligonucleotide
agtactatgt

SEQ ID NO: 851 - DNA, 10 bp, Artificial
  Oligonucleotide
atcgagagta

SEQ ID NO: 852 - DNA, 10 bp, Artificial
  Oligonucleotide
cagcgtgtac

SEQ ID NO: 853 - DNA, 10 bp, Artificial
  Oligonucleotide
cgagagcgcg

SEQ ID NO: 854 - DNA, 10 bp, Artificial
  Oligonucleotide
cgtgtctcta

SEQ ID NO: 855 - DNA, 10 bp, Artificial
  Oligonucleotide
ctcacacgac

SEQ ID NO: 856 - DNA, 10 bp, Artificial
  Oligonucleotide
tacagatagc

SEQ ID NO: 857 - DNA, 10 bp, Artificial
  Oligonucleotide
tagtcacgca

SEQ ID NO: 858 - DNA, 10 bp, Artificial
  Oligonucleotide
tctactgctc

SEQ ID NO: 859 - DNA, 10 bp, Artificial
  Oligonucleotide
tgcgcgtcgt

SEQ ID NO: 860 - DNA, 10 bp, Artificial
  Oligonucleotide
tgtatcagag

SEQ ID NO: 861 - DNA, 10 bp, Artificial
  Oligonucleotide
actctatcac

SEQ ID NO: 862 - DNA, 10 bp, Artificial
  Oligonucleotide
actgatagtg

SEQ ID NO: 863 - DNA, 10 bp, Artificial
  Oligonucleotide
agacgcactc

SEQ ID NO: 864 - DNA, 10 bp, Artificial
  Oligonucleotide
agtagtgaga

SEQ ID NO: 865 - DNA, 10 bp, Artificial
  Oligonucleotide
cagcgtgtac

SEQ ID NO: 866 - DNA, 10 bp, Artificial
  Oligonucleotide
cgagagcgcg

SEQ ID NO: 867 - DNA, 10 bp, Artificial
  Oligonucleotide
cgtgtctcta

SEQ ID NO: 868 - DNA, 10 bp, Artificial
  Oligonucleotide
ctctcgagag

SEQ ID NO: 869 - DNA, 10 bp, Artificial
  Oligonucleotide
tacagacgac

SEQ ID NO: 870 - DNA, 10 bp, Artificial
  Oligonucleotide
tagtagatct

SEQ ID NO: 871 - DNA, 10 bp, Artificial
  Oligonucleotide
tatcactgcg

SEQ ID NO: 872 - DNA, 10 bp, Artificial
  Oligonucleotide
tctacacata

SEQ ID NO: 873 - DNA, 10 bp, Artificial
  Oligonucleotide
tgatgtatag

SEQ ID NO: 874 - DNA, 10 bp, Artificial
  Oligonucleotide
tgcgcgtcgt

SEQ ID NO: 875 - DNA, 10 bp, Artificial
  Oligonucleotide
actctatcac

SEQ ID NO: 876 - DNA, 10 bp, Artificial
  Oligonucleotide
actgatagtg

SEQ ID NO: 877 - DNA, 10 bp, Artificial
  Oligonucleotide
agacgcactc

SEQ ID NO: 878 - DNA, 10 bp, Artificial
  Oligonucleotide
agtagtgaga

SEQ ID NO: 879 - DNA, 10 bp, Artificial
  Oligonucleotide
cagcgtgtac

SEQ ID NO: 880 - DNA, 10 bp, Artificial
  Oligonucleotide
cgagagcgcg

SEQ ID NO: 881 - DNA, 10 bp, Artificial
  Oligonucleotide
cgtgtctcta

SEQ ID NO: 882 - DNA, 10 bp, Artificial
  Oligonucleotide
ctctcgagag

SEQ ID NO: 883 - DNA, 10 bp, Artificial
  Oligonucleotide
tacactgctg

SEQ ID NO: 884 - DNA, 10 bp, Artificial
  Oligonucleotide
tacagacgac

SEQ ID NO: 885 - DNA, 10 bp, Artificial
  Oligonucleotide
tagtagatct

SEQ ID NO: 886 - DNA, 10 bp, Artificial
  Oligonucleotide
tctacacata

SEQ ID NO: 887 - DNA, 10 bp, Artificial
  Oligonucleotide
tgatgtatag

SEQ ID NO: 888 - DNA, 10 bp, Artificial
  Oligonucleotide
tgcgcgtcgt

SEQ ID NO: 889 - DNA, 10 bp, Artificial
  Oligonucleotide
acgcatctac

SEQ ID NO: 890 - DNA, 10 bp, Artificial
  Oligonucleotide
actcgcgctg

SEQ ID NO: 891 - DNA, 10 bp, Artificial
  Oligonucleotide
agactgtgtc

SEQ ID NO: 892 - DNA, 10 bp, Artificial
  Oligonucleotide
atatacgcac

SEQ ID NO: 893 - DNA, 10 bp, Artificial
  Oligonucleotide
cacgtagtga

SEQ ID NO: 894 - DNA, 10 bp, Artificial
  Oligonucleotide
cgagagcgcg

SEQ ID NO: 895 - DNA, 10 bp, Artificial
  Oligonucleotide
cgtcgcagac

SEQ ID NO: 896 - DNA, 10 bp, Artificial
  Oligonucleotide
cgtgtctcta

SEQ ID NO: 897 - DNA, 10 bp, Artificial
  Oligonucleotide
ctctacatcg

SEQ ID NO: 898 - DNA, 10 bp, Artificial
  Oligonucleotide
tacagatagc

SEQ ID NO: 899 - DNA, 10 bp, Artificial
  Oligonucleotide
tagtctgaca

SEQ ID NO: 900 - DNA, 10 bp, Artificial
  Oligonucleotide
tctatcgagt

SEQ ID NO: 901 - DNA, 10 bp, Artificial
  Oligonucleotide
tgatatactg

SEQ ID NO: 902 - DNA, 10 bp, Artificial
  Oligonucleotide
tgcgcgtcgt

SEQ ID NO: 903 - DNA, 10 bp, Artificial
  Oligonucleotide
actcgtgtga

SEQ ID NO: 904 - DNA, 10 bp, Artificial
  Oligonucleotide
actgacacgt

SEQ ID NO: 905 - DNA, 10 bp, Artificial
  Oligonucleotide
agcactactc

SEQ ID NO: 906 - DNA, 10 bp, Artificial
  Oligonucleotide
atacagatac

SEQ ID NO: 907 - DNA, 10 bp, Artificial
  Oligonucleotide
atatgtcgtc

SEQ ID NO: 908 - DNA, 10 bp, Artificial
  Oligonucleotide
cgagagcgcg

SEQ ID NO: 909 - DNA, 10 bp, Artificial
  Oligonucleotide
cgtcgcagac

SEQ ID NO: 910 - DNA, 10 bp, Artificial
  Oligonucleotide
cgtgtctcta

SEQ ID NO: 911 - DNA, 10 bp, Artificial
  Oligonucleotide
ctctacgtat

SEQ ID NO: 912 - DNA, 10 bp, Artificial
  Oligonucleotide
tagtcacgca

SEQ ID NO: 913 - DNA, 10 bp, Artificial
  Oligonucleotide
tcacactaga

SEQ ID NO: 914 - DNA, 10 bp, Artificial
  Oligonucleotide
tctatctgcg

SEQ ID NO: 915 - DNA, 10 bp, Artificial
  Oligonucleotide
tgatgtatag

SEQ ID NO: 916 - DNA, 10 bp, Artificial
  Oligonucleotide
tgcgcgtcgt

SEQ ID NO: 917 - DNA, 10 bp, Artificial
  Oligonucleotide
actcgtgtga

SEQ ID NO: 918 - DNA, 10 bp, Artificial
  Oligonucleotide
actgctcacg

SEQ ID NO: 919 - DNA, 10 bp, Artificial
  Oligonucleotide
agtatacagt

SEQ ID NO: 920 - DNA, 10 bp, Artificial
  Oligonucleotide
atacagtaca

SEQ ID NO: 921 - DNA, 10 bp, Artificial
  Oligonucleotide
atagtcgatc

SEQ ID NO: 922 - DNA, 10 bp, Artificial
  Oligonucleotide
cacgatactc

SEQ ID NO: 923 - DNA, 10 bp, Artificial
  Oligonucleotide
cgagagcgcg

SEQ ID NO: 924 - DNA, 10 bp, Artificial
  Oligonucleotide
cgtcgcagac

SEQ ID NO: 925 - DNA, 10 bp, Artificial
  Oligonucleotide
cgtgtctcta

SEQ ID NO: 926 - DNA, 10 bp, Artificial
  Oligonucleotide
ctctacgtat

SEQ ID NO: 927 - DNA, 10 bp, Artificial
  Oligonucleotide
tatctagcgc

SEQ ID NO: 928 - DNA, 10 bp, Artificial
  Oligonucleotide
tatgagatag

SEQ ID NO: 929 - DNA, 10 bp, Artificial
  Oligonucleotide
tcacactgag

SEQ ID NO: 930 - DNA, 10 bp, Artificial
  Oligonucleotide
tgcgcgtcgt

SEQ ID NO: 931 - DNA, 10 bp, Artificial
  Oligonucleotide
actcgtgtga

SEQ ID NO: 932 - DNA, 10 bp, Artificial
  Oligonucleotide
actgctcacg

SEQ ID NO: 933 - DNA, 10 bp, Artificial
  Oligonucleotide
agtatacagt

SEQ ID NO: 934 - DNA, 10 bp, Artificial
  Oligonucleotide
atagtcgatc

SEQ ID NO: 935 - DNA, 10 bp, Artificial
  Oligonucleotide
cacgatactc

SEQ ID NO: 936 - DNA, 10 bp, Artificial
  Oligonucleotide
cgagagcgcg

SEQ ID NO: 937 - DNA, 10 bp, Artificial
  Oligonucleotide
cgtcgcagac

SEQ ID NO: 938 - DNA, 10 bp, Artificial
  Oligonucleotide
cgtgtctcta

SEQ ID NO: 939 - DNA, 10 bp, Artificial
  Oligonucleotide
ctctacgtat

SEQ ID NO: 940 - DNA, 10 bp, Artificial
  Oligonucleotide
tatctagcgc

SEQ ID NO: 941 - DNA, 10 bp, Artificial
  Oligonucleotide
tatgagatag

SEQ ID NO: 942 - DNA, 10 bp, Artificial
  Oligonucleotide
tcacactaga

SEQ ID NO: 943 - DNA, 10 bp, Artificial
  Oligonucleotide
tcagtatgtg

SEQ ID NO: 944 - DNA, 10 bp, Artificial
  Oligonucleotide
tgcgcgtcgt

SEQ ID NO: 945 - DNA, 10 bp, Artificial
  Oligonucleotide
acatactcac

SEQ ID NO: 946 - DNA, 10 bp, Artificial
  Oligonucleotide
acgcacgata

SEQ ID NO: 947 - DNA, 10 bp, Artificial
  Oligonucleotide
actgtgtgag

SEQ ID NO: 948 - DNA, 10 bp, Artificial
  Oligonucleotide
agtatacagt

SEQ ID NO: 949 - DNA, 10 bp, Artificial
  Oligonucleotide
atacgtatgc

SEQ ID NO: 950 - DNA, 10 bp, Artificial
  Oligonucleotide
cgagagcgcg

SEQ ID NO: 951 - DNA, 10 bp, Artificial
  Oligonucleotide
cgtcgcagac

SEQ ID NO: 952 - DNA, 10 bp, Artificial
  Oligonucleotide
cgtgtctcta

SEQ ID NO: 953 - DNA, 10 bp, Artificial
  Oligonucleotide
ctctacatcg

SEQ ID NO: 954 - DNA, 10 bp, Artificial
  Oligonucleotide
tacactgctg

SEQ ID NO: 955 - DNA, 10 bp, Artificial
  Oligonucleotide
tatgagactc

SEQ ID NO: 956 - DNA, 10 bp, Artificial
  Oligonucleotide
tcagcgtaca

SEQ ID NO: 957 - DNA, 10 bp, Artificial
  Oligonucleotide
tctcgcgcgt

SEQ ID NO: 958 - DNA, 10 bp, Artificial
  Oligonucleotide
tgactagtat

SEQ ID NO: 959 - DNA, 10 bp, Artificial
  Oligonucleotide
acgctagact

SEQ ID NO: 960 - DNA, 10 bp, Artificial
  Oligonucleotide
agtcgtgaga

SEQ ID NO: 961 - DNA, 10 bp, Artificial
  Oligonucleotide
atatcatacg

SEQ ID NO: 962 - DNA, 10 bp, Artificial
  Oligonucleotide
catagtagtg

SEQ ID NO: 963 - DNA, 10 bp, Artificial
  Oligonucleotide
cgagagatac

SEQ ID NO: 964 - DNA, 10 bp, Artificial
  Oligonucleotide
cgtgtctcta

SEQ ID NO: 965 - DNA, 10 bp, Artificial
  Oligonucleotide
ctcgcgtgtc

SEQ ID NO: 966 - DNA, 10 bp, Artificial
  Oligonucleotide
ctctacgcag

SEQ ID NO: 967 - DNA, 10 bp, Artificial
  Oligonucleotide
tacacacata

SEQ ID NO: 968 - DNA, 10 bp, Artificial
  Oligonucleotide
tactgtgtat

SEQ ID NO: 969 - DNA, 10 bp, Artificial
  Oligonucleotide
tagctctcgc

SEQ ID NO: 970 - DNA, 10 bp, Artificial
  Oligonucleotide
tctagatcga

SEQ ID NO: 971 - DNA, 10 bp, Artificial
  Oligonucleotide
tgacagcgcg

SEQ ID NO: 972 - DNA, 10 bp, Artificial
  Oligonucleotide
tgagtacagt

SEQ ID NO: 973 - DNA, 10 bp, Artificial
  Oligonucleotide
acgagtgcgt

SEQ ID NO: 974 - DNA, 10 bp, Artificial
  Oligonucleotide
acgctcgaca

SEQ ID NO: 975 - DNA, 10 bp, Artificial
  Oligonucleotide
agacgcactc

SEQ ID NO: 976 - DNA, 10 bp, Artificial
  Oligonucleotide
agcactgtag

SEQ ID NO: 977 - DNA, 10 bp, Artificial
  Oligonucleotide
atatcgcgag

SEQ ID NO: 978 - DNA, 10 bp, Artificial
  Oligonucleotide
atcagacacg

SEQ ID NO: 979 - DNA, 10 bp, Artificial
  Oligonucleotide
catagtagtg

SEQ ID NO: 980 - DNA, 10 bp, Artificial
  Oligonucleotide
cgagagatac

SEQ ID NO: 981 - DNA, 10 bp, Artificial
  Oligonucleotide
cgtgtctcta

SEQ ID NO: 982 - DNA, 10 bp, Artificial
  Oligonucleotide
ctcgcgtgtc

SEQ ID NO: 983 - DNA, 10 bp, Artificial
  Oligonucleotide
tactgagcta

SEQ ID NO: 984 - DNA, 10 bp, Artificial
  Oligonucleotide
tagtatcagc

SEQ ID NO: 985 - DNA, 10 bp, Artificial
  Oligonucleotide
tctctatgcg

SEQ ID NO: 986 - DNA, 10 bp, Artificial
  Oligonucleotide
tgatacgtct

SEQ ID NO: 987 - DNA, 10 bp, Artificial
  Oligonucleotide
acactgtata

SEQ ID NO: 988 - DNA, 10 bp, Artificial
  Oligonucleotide
acagcgacgt

SEQ ID NO: 989 - DNA, 10 bp, Artificial
  Oligonucleotide
acatactcac

SEQ ID NO: 990 - DNA, 10 bp, Artificial
  Oligonucleotide
atcgcgctag

SEQ ID NO: 991 - DNA, 10 bp, Artificial
  Oligonucleotide
atgtatagtc

SEQ ID NO: 992 - DNA, 10 bp, Artificial
  Oligonucleotide
cgacatcgtg

SEQ ID NO: 993 - DNA, 10 bp, Artificial
  Oligonucleotide
cgagagatac

SEQ ID NO: 994 - DNA, 10 bp, Artificial
  Oligonucleotide
cgtgtctcta

SEQ ID NO: 995 - DNA, 10 bp, Artificial
  Oligonucleotide
ctctacgaga

SEQ ID NO: 996 - DNA, 10 bp, Artificial
  Oligonucleotide
tactagatct

SEQ ID NO: 997 - DNA, 10 bp, Artificial
  Oligonucleotide
tagatgtgag

SEQ ID NO: 998 - DNA, 10 bp, Artificial
  Oligonucleotide
tctgtcagcg

SEQ ID NO: 999 - DNA, 10 bp, Artificial
  Oligonucleotide
tgcgcgtgtc

SEQ ID NO: 1000 - DNA, 10 bp, Artificial
  Oligonucleotide
tgtcgtacat

SEQ ID NO: 1001 - DNA, 10 bp, Artificial
  Oligonucleotide
acactgtata

SEQ ID NO: 1002 - DNA, 10 bp, Artificial
  Oligonucleotide
agacactcgt

SEQ ID NO: 1003 - DNA, 10 bp, Artificial
  Oligonucleotide
atcgcgctag

SEQ ID NO: 1004 - DNA, 10 bp, Artificial
  Oligonucleotide
atgtatagtc

SEQ ID NO: 1005 - DNA, 10 bp, Artificial
  Oligonucleotide
cgagagatac

SEQ ID NO: 1006 - DNA, 10 bp, Artificial
  Oligonucleotide
cgtctgagcg

SEQ ID NO: 1007 - DNA, 10 bp, Artificial
  Oligonucleotide
cgtgtctcta

SEQ ID NO: 1008 - DNA, 10 bp, Artificial
  Oligonucleotide
ctacgtcgac

SEQ ID NO: 1009 - DNA, 10 bp, Artificial
  Oligonucleotide
tactagctca

SEQ ID NO: 1010 - DNA, 10 bp, Artificial
  Oligonucleotide
tagatgtgag

SEQ ID NO: 1011 - DNA, 10 bp, Artificial
  Oligonucleotide
tcagtcacgc

SEQ ID NO: 1012 - DNA, 10 bp, Artificial
  Oligonucleotide
tctacagagt

SEQ ID NO: 1013 - DNA, 10 bp, Artificial
  Oligonucleotide
tgcgcgtgtc

SEQ ID NO: 1014 - DNA, 10 bp, Artificial
  Oligonucleotide
tgtcgtacat

SEQ ID NO: 1015 - DNA, 10 bp, Artificial
  Oligonucleotide
actctcgaca

SEQ ID NO: 1016 - DNA, 10 bp, Artificial
  Oligonucleotide
agatacactg

SEQ ID NO: 1017 - DNA, 10 bp, Artificial
  Oligonucleotide
atacgtatct

SEQ ID NO: 1018 - DNA, 10 bp, Artificial
  Oligonucleotide
atgtgacgtc

SEQ ID NO: 1019 - DNA, 10 bp, Artificial
  Oligonucleotide
cacgactgag

SEQ ID NO: 1020 - DNA, 10 bp, Artificial
  Oligonucleotide
cgagagatac

SEQ ID NO: 1021 - DNA, 10 bp, Artificial
  Oligonucleotide
cgcacgtcgt

SEQ ID NO: 1022 - DNA, 10 bp, Artificial
  Oligonucleotide
cgtgtctcta

SEQ ID NO: 1023 - DNA, 10 bp, Artificial
  Oligonucleotide
ctgtatagcg

SEQ ID NO: 1024 - DNA, 10 bp, Artificial
  Oligonucleotide
tagatgtgta

SEQ ID NO: 1025 - DNA, 10 bp, Artificial
  Oligonucleotide
tatcagtcac

SEQ ID NO: 1026 - DNA, 10 bp, Artificial
  Oligonucleotide
tcgactactc

SEQ ID NO: 1027 - DNA, 10 bp, Artificial
  Oligonucleotide
tcgtagcaga

SEQ ID NO: 1028 - DNA, 10 bp, Artificial
  Oligonucleotide
tgagcgcgcg

SEQ ID NO: 1029 - DNA, 10 bp, Artificial
  Oligonucleotide
acagctacag

SEQ ID NO: 1030 - DNA, 10 bp, Artificial
  Oligonucleotide
agctctgtcg

SEQ ID NO: 1031 - DNA, 10 bp, Artificial
  Oligonucleotide
atacgtgctc

SEQ ID NO: 1032 - DNA, 10 bp, Artificial
  Oligonucleotide
atgtatacgt

SEQ ID NO: 1033 - DNA, 10 bp, Artificial
  Oligonucleotide
cacgagtgtg

SEQ ID NO: 1034 - DNA, 10 bp, Artificial
  Oligonucleotide
cgagagatac

SEQ ID NO: 1035 - DNA, 10 bp, Artificial
  Oligonucleotide
cgtgtctcta

SEQ ID NO: 1036 - DNA, 10 bp, Artificial
  Oligonucleotide
ctcacgtaga

SEQ ID NO: 1037 - DNA, 10 bp, Artificial
  Oligonucleotide
tagacactga

SEQ ID NO: 1038 - DNA, 10 bp, Artificial
  Oligonucleotide
tatctagata

SEQ ID NO: 1039 - DNA, 10 bp, Artificial
  Oligonucleotide
tcatatcgcg

SEQ ID NO: 1040 - DNA, 10 bp, Artificial
  Oligonucleotide
tctgtgtgac

SEQ ID NO: 1041 - DNA, 10 bp, Artificial
  Oligonucleotide
tgcgtcgagt

SEQ ID NO: 1042 - DNA, 10 bp, Artificial
  Oligonucleotide
tgtcgcacac

SEQ ID NO: 1043 - DNA, 10 bp, Artificial
  Oligonucleotide
acacacacgt

SEQ ID NO: 1044 - DNA, 10 bp, Artificial
  Oligonucleotide
actctcgatc

SEQ ID NO: 1045 - DNA, 10 bp, Artificial
  Oligonucleotide
agcgcgtgtg

SEQ ID NO: 1046 - DNA, 10 bp, Artificial
  Oligonucleotide
atgactagcg

SEQ ID NO: 1047 - DNA, 10 bp, Artificial
  Oligonucleotide
cgagagatac

SEQ ID NO: 1048 - DNA, 10 bp, Artificial
  Oligonucleotide
cgtcatgacg

SEQ ID NO: 1049 - DNA, 10 bp, Artificial
  Oligonucleotide
cgtgtctcta

SEQ ID NO: 1050 - DNA, 10 bp, Artificial
  Oligonucleotide
ctacgtctgc

SEQ ID NO: 1051 - DNA, 10 bp, Artificial
  Oligonucleotide
tacagcgcta

SEQ ID NO: 1052 - DNA, 10 bp, Artificial
  Oligonucleotide
tagtcagtat

SEQ ID NO: 1053 - DNA, 10 bp, Artificial
  Oligonucleotide
tcactgtaga

SEQ ID NO: 1054 - DNA, 10 bp, Artificial
  Oligonucleotide
tctgtgcgcg

SEQ ID NO: 1055 - DNA, 10 bp, Artificial
  Oligonucleotide
tgatactgtc

SEQ ID NO: 1056 - DNA, 10 bp, Artificial
  Oligonucleotide
tgtcgcacac

SEQ ID NO: 1057 - DNA, 10 bp, Artificial
  Oligonucleotide
acacacacgt

SEQ ID NO: 1058 - DNA, 10 bp, Artificial
  Oligonucleotide
actctcgatc

SEQ ID NO: 1059 - DNA, 10 bp, Artificial
  Oligonucleotide
agcgcgtgtg

SEQ ID NO: 1060 - DNA, 10 bp, Artificial
  Oligonucleotide
atgactagcg

SEQ ID NO: 1061 - DNA, 10 bp, Artificial
  Oligonucleotide
cgagagatac

SEQ ID NO: 1062 - DNA, 10 bp, Artificial
  Oligonucleotide
cgtcatgacg

SEQ ID NO: 1063 - DNA, 10 bp, Artificial
  Oligonucleotide
cgtgtctcta

SEQ ID NO: 1064 - DNA, 10 bp, Artificial
  Oligonucleotide
ctacgtctgc

SEQ ID NO: 1065 - DNA, 10 bp, Artificial
  Oligonucleotide
tacagcgcta

SEQ ID NO: 1066 - DNA, 10 bp, Artificial
  Oligonucleotide
tagtcagtat

SEQ ID NO: 1067 - DNA, 10 bp, Artificial
  Oligonucleotide
tcgatgtaga

SEQ ID NO: 1068 - DNA, 10 bp, Artificial
  Oligonucleotide
tctgtgcgcg

SEQ ID NO: 1069 - DNA, 10 bp, Artificial
  Oligonucleotide
tgatactgtc

SEQ ID NO: 1070 - DNA, 10 bp, Artificial
  Oligonucleotide
tgtcgcacac

SEQ ID NO: 1071 - DNA, 10 bp, Artificial
  Oligonucleotide
acacacacgt

SEQ ID NO: 1072 - DNA, 10 bp, Artificial
  Oligonucleotide
actctcgatc

SEQ ID NO: 1073 - DNA, 10 bp, Artificial
  Oligonucleotide
agcgcgtgtg

SEQ ID NO: 1074 - DNA, 10 bp, Artificial
  Oligonucleotide
atgactagcg

SEQ ID NO: 1075 - DNA, 10 bp, Artificial
  Oligonucleotide
cgagagatac

SEQ ID NO: 1076 - DNA, 10 bp, Artificial
  Oligonucleotide
cgtcatgacg

SEQ ID NO: 1077 - DNA, 10 bp, Artificial
  Oligonucleotide
cgtgtctcta

SEQ ID NO: 1078 - DNA, 10 bp, Artificial
  Oligonucleotide
ctacgtctgc

SEQ ID NO: 1079 - DNA, 10 bp, Artificial
  Oligonucleotide
tacagcgcta

SEQ ID NO: 1080 - DNA, 10 bp, Artificial
  Oligonucleotide
tagtcagtat

SEQ ID NO: 1081 - DNA, 10 bp, Artificial
  Oligonucleotide
tctatataga

SEQ ID NO: 1082 - DNA, 10 bp, Artificial
  Oligonucleotide
tctgtgcgcg

SEQ ID NO: 1083 - DNA, 10 bp, Artificial
  Oligonucleotide
tgatactgtc

SEQ ID NO: 1084 - DNA, 10 bp, Artificial
  Oligonucleotide
tgtcgcacac

SEQ ID NO: 1085 - DNA, 10 bp, Artificial
  Oligonucleotide
acacacacgt

SEQ ID NO: 1086 - DNA, 10 bp, Artificial
  Oligonucleotide
actctcgatc

SEQ ID NO: 1087 - DNA, 10 bp, Artificial
  Oligonucleotide
agcgcgtgtg

SEQ ID NO: 1088 - DNA, 10 bp, Artificial
  Oligonucleotide
atgactagcg

SEQ ID NO: 1089 - DNA, 10 bp, Artificial
  Oligonucleotide
cgagagatac

SEQ ID NO: 1090 - DNA, 10 bp, Artificial
  Oligonucleotide
cgtcatgacg

SEQ ID NO: 1091 - DNA, 10 bp, Artificial
  Oligonucleotide
cgtgtctcta

SEQ ID NO: 1092 - DNA, 10 bp, Artificial
  Oligonucleotide
ctacgtctgc

SEQ ID NO: 1093 - DNA, 10 bp, Artificial
  Oligonucleotide
tacagcgcta

SEQ ID NO: 1094 - DNA, 10 bp, Artificial
  Oligonucleotide
tagtcagtat

SEQ ID NO: 1095 - DNA, 10 bp, Artificial
  Oligonucleotide
tatatataga

SEQ ID NO: 1096 - DNA, 10 bp, Artificial
  Oligonucleotide
tctgtgcgcg

SEQ ID NO: 1097 - DNA, 10 bp, Artificial
  Oligonucleotide
tgatactgtc

SEQ ID NO: 1098 - DNA, 10 bp, Artificial
  Oligonucleotide
tgtcgcacac